(19) United States
(12) Patent Application Publication
(10) Pub. No.: US 2010/0145006 A1
(43) Pub. Date: Jun. 10, 2010

(54) SUPRAVALENT COMPOUNDS

(76) Inventors: Hans-Georg Frank, Kerkrade (NL); Udo Haberl, Baesweiler (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/917,991

(22) PCT Filed: Jun. 23, 2006
(86) PCT No.: PCT/EP06/06097

§ 371 Date: Feb. 26, 2008

Related U.S. Application Data
(60) Provisional application No. 60/780,568, filed on Mar. 9, 2006.
(60) Provisional application No. 60/747,515, filed on May 17, 2006.

(30) Foreign Application Priority Data
Jun. 23, 2005 (EP) 05013594.6
Sep. 14, 2005 (EP) 05020035.1
Nov. 10, 2005 (EP) EP0512075
Dec. 24, 2005 (EP) 05028310.0
Mar. 9, 2006 (EP) 06004833.7
May 17, 2006 (EP) 06010174.805

(51) Int. Cl. C07K 2/00

(52) U.S. Cl. 530/323; 530/350

(57) ABSTRACT

Supravalent peptide compounds depicting enhanced efficacy, and which comprise at least bivalent peptide units that bind to a receptor target and are connected to a polymeric carrier unit are disclosed herein.

The present invention pertains to peptide compounds.

With the development of research on proteins, a great number of peptides having various biological and pharmaceutical activities have been found. Several of these peptides execute their actions via the binding to target molecules as for example receptor molecules (such as cytokine receptors).

Hematopoietic growth factors (HGFs) have proved to be clinically successful therapeutics; however, their size (15-70 kDa), conformational instability, susceptibility to proteolytic degradation, poor membrane penetration, antigenicity, high cost of production, and unfavourable pharmacokinetics can make them less than ideal drug candidates. Furthermore, the poor bioavailability of the native proteins requires that they be administered parenterally. It is advantageous, therefore, to develop small-molecule agonists (and antagonists) of HGF receptors that are equipotent to their polypeptide counterparts but that lack some of the inherent drawbacks of large proteins. The identification and examination of smaller peptides that bind to and activate cytokine receptors also provides a better understanding of ligand-receptor interactions. This information is used to design orally available small-molecule cytokine mimetics rationally. Activation of transmembrane receptors by growth factors and cytokines occurs when a ligand binds to a specific domain on the receptor, thereby inducing a conformational change and triggering dimerization or oligomerization of receptor chains. Upon ligand binding, several members of the class I cytokine receptors form homodimers, including the erythropoietin receptor (EPOR), thrombopoietin receptor (TPOR), granulocyte colony-stimulating factor receptor (G-CSFR), growth hormone receptor (GHR), and prolactin receptor (PrR). Several studies have been reported that are directed toward discovering the precise details of the dimerization interfaces and the degree to which the unliganded receptors exist as dimers. The results of these studies have shown structural and functional similarities between the class I cytokine receptors. Studies also have shown that receptor dimerization alone, although necessary for intracellular signaling, is not sufficient to produce signal transduction. Recent reports have shown that both small molecules and peptides can bind to and activate homodimeric cytokine receptors by acting as agonists and mimicking the effects of the natural proteins (see Labor, E. G. (2004). Small-Molecule and Peptide Agonists: A Literature Review. Hematopoietic Growth Factors in Oncology—Basic Science and Clinical Therapeutics. G. Morstyn, M. Foote and G. J. Lieschke: 65-80). However, their biological activity is often inferior to the natural molecules. Consequently, attempts are made to improve the biological activity of the mimetic molecules.

Successful examples of such peptides include peptides binding the erythropoietin receptor and mimicking the function of erythropoietin and peptides binding the thrombopoietin receptor and mimicking the function of thrombopoietin.

The hormone erythropoietin (EPO) is a glycoprotein constituted by 165 amino acids and having four glycosylation sites. It stimulates mitotic division and the differentiation of erythrocytes precursor cells and thus ensures the production of erythrocytes. Since the use of EPO or recombinant EPO has several disadvantages including immunogenic reactions, synthetic peptides are used, which do not share any sequence homology or structural relationship with EPO but anyhow bind and interact with the EPO-R (see e.g. Wrighton et al., 1996). Synthetic peptides mimicking EPO's activity (“EPO mimetic peptides”) are in the meantime well known in the state of the art (see e.g. WO 96/40772; WO 96/40749; WO 01/38342; WO 01/091780; WO 2004/101611; WO 2004/100997; WO 2004/101600; WO 2004/101606).

EPO and EPO mimetic peptides activate the EPO receptor by binding the extracellular domains of the receptor and presumable dimerizing two receptor monomers to a receptor complex thereby initiating signal transduction (Johnson et al., 1997). The crystal structure of the EPO receptor bound to EMP1 (a well known EPO mimetic peptide) revealed the formation of a receptor-peptide complex consisting of two peptides bound to two receptor monomers. Thus, it was not really a surprise that combination of exactly two of these binding domains in one single molecule enhanced the activity considerably, leading to the result that peptides with one single binding domain showed the same qualitative pattern of activity while two of the binding domains joint together show a much lower ED50 (Effect Dose 50%, a measure of activity).

Preparation methods for respective peptide dimers of e.g. EPO or TPO mimetic peptides are also well known in the state of the art and range from e.g. dimerization via PEGylation, disulfide bridges or lysine side chains (see e.g. WO 96/40772; WO 96/40749; WO 01/38342; WO 01/091780; WO 2004/101611; WO 2004/100997; WO 2004/101600; WO 2004/101606, Wrighton et al., 1997; Johnson et al., 1997; WO 98/25965). All these methods combine monomeric peptides via a linker structure in order to obtain the desired dimeric or even multimeric molecules which enhance the formation of the active dimeric or even multimeric receptor complex.

A similar concept for combining monomeric units is also known for other binding molecules (see for example WO 2004/014951). In order to generate a molecule that is able to interact with the respective di- or multimeric receptor complex, this application teaches to use a small support structure in order to connect the monomeric receptor binding domains in a spatial relationship that allows the interaction with the respective receptor complexes (and e.g. inducing di- or trimerization of the receptor).

However, even though the dimerization (or even multimerization in case of a multimeric receptor) of the monomeric peptide units usually improves the activity compared to the respective monomeric peptides, it is desirable to further enhance the activity. For example even the dimeric EPO mimetic peptides are still far less potent than the EPO molecule regarding the activation of the cellular mechanisms.

It is also known in the state of the art to couple one or more hydrophilic carrier units (such as e.g. PEG) to a peptide. It has been found that when peptides are derivatised with a hydrophilic polymer, their solubility and circulation half-live is increased and their immuogenicity is decreased (see e.g. WO 98/25965). However, it has also been reported that the attachment of such a hydrophilic carrier may decrease the biological activity. An increase of biological activity was not reported.

Several approaches were made in order to increase the activity of the peptides, for example by variation of the amino acid sequence in order to identify more potent candidates. However, so far no appropriate solution for enhancing the activity of peptides, especially of EPO or TPO mimetic peptides, is known in the state of the art.

It is thus the object of the present invention to provide peptide compounds binding to a receptor target and having an increased activity.

The object is solved by a compound that binds target molecules and comprises
i) at least two peptide units wherein each peptide unit comprises at least two domains with a binding capacity to the target (and hence at least two monomeric binding units);
ii) at least one polymeric carrier unit;
   wherein said peptide units are bound to said polymeric carrier unit.

Surprisingly, it has been found that the combination of two or more bi- or multivalent peptides on a polymeric support is greatly increasing the efficacy of the bivalent (or even multivalent) peptides to bind the respective target, which is usually a receptor molecule not only additively, but even over-additively. Thus a synergistic effect on binding efficacy is observed.

The term “bivalent” as used for the purpose of the present invention is defined as a peptide comprising two domains with a binding capacity to a target, which is usually a receptor (this term will thus be used hereinafter). The term “bivalent” is used interchangeably with the term “dimeric”. Accordingly, a “multivalent” or “multimeric” peptide has several respective binding domains and thus monomeric binding units. It is self-evident that the terms “peptide” and “peptide unit” do not incorporate any restrictions regarding size and incorporate oligo- and polypeptides as well as proteins. However, it is preferred that the peptide units coupled to the carrier unit have a length of about 200 amino acids or less, or of about 150 amino acids or less, more preferred about 100 amino acids or even 50 amino acids and less.

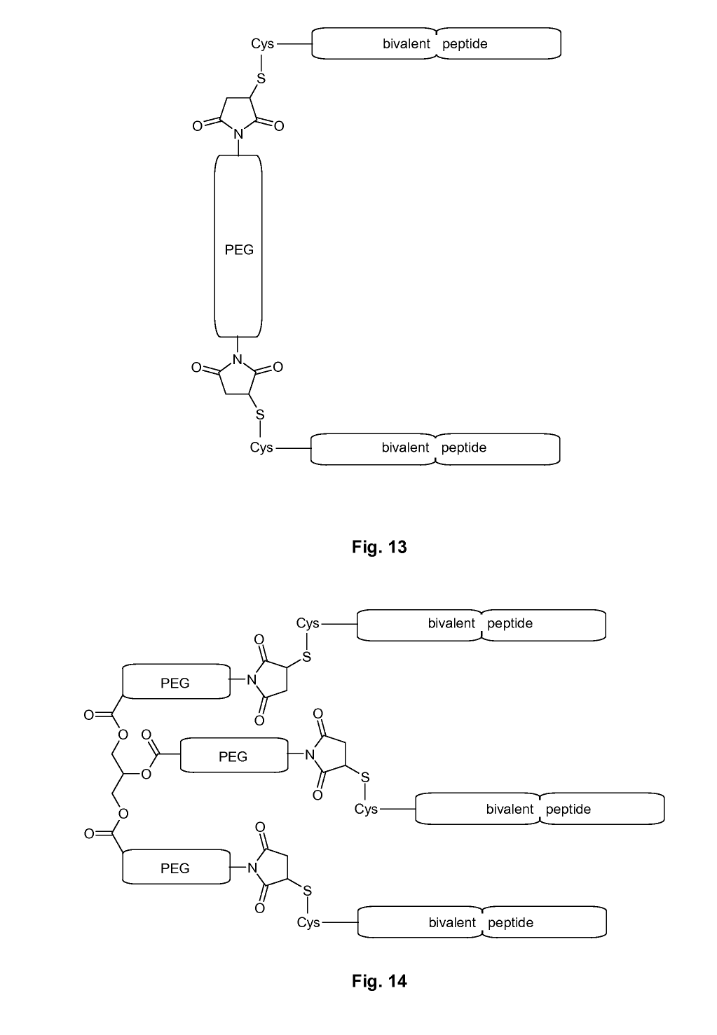

Compounds comprising two or more bi- or multivalent peptide units attached to a polymeric carrier unit are named “supravalent” in the context of the present invention. These supravalent molecules greatly differ from the dimeric or multimeric molecules known in the state of the art. The state of the art combines several merely monomeric peptide units in order to create a dimer or multimer. In contrast, the supravalent molecules are generated by connecting already (at least) bivalent peptide units to a polymeric carrier unit thereby creating a supravalent molecule carrying several di- or multimeric peptide units (illustrated examples of this concept are given in FIGS. 13 to 15). Thereby the overall activity and efficacy of the di- or multimeric peptides is greatly enhanced thus decreasing the EC50 dose.

So far the reasons for the great potency of the supravalent molecules compared to the bi- or multimeric molecules known in the state of the art are not fully understood. It might be due to the fact that the dimeric molecules known in the state of the art (e.g. dimeric EPO mimetic peptides) provide merely one target respectively one active receptor binding unit per compound molecule. Thus only one (dimeric) receptor complex is generated upon binding of the dimeric compound thereby inducing only one signal transduction process in the cell. For example two monomeric EPO mimetic peptides are connected via PEG to form a peptide dimer thereby facilitating dimerization of the receptor monomers necessary for signal transduction (Johnson et al., 1997).

In contrast, the supravalent compounds according to the invention comprise several already di- or multimeric receptor binding units. Supravalent compounds according to the present invention thus Carry several (bi- or multivalent) receptor binding units. Each di- or multimeric peptide unit coupled to the carrier represents one receptor binding unit. This might allow the generation of several receptor complexes on the cell surface per compound molecule thereby inducing (or blocking in case of an antagonist) several signal transductions and thereby patencing the activity of the peptide units over-additively. Binding of the supravalent compounds might result in a clustering of receptor complexes on the cell-surface.

The peptide units according to the invention are either home- or heterogenic, meaning that either identical or differing peptide units are coupled to the polymeric carrier. The same applies to the binding domains of the peptide units which can also be home- or heterogenic. Homogenic binding domains (monomers) are preferred in case a target receptor is bound that is composed of identical protein subunits (such as e.g. the homodimeric EPO receptor). However, the amino acid sequence of the homogenic binding domains may still vary even though they bind the same receptor target (and are thus functionally homogenic). Heterogenic binding domains (monomers) are preferred in case a target receptor is bound that is composed of differing protein subunits (such as e.g. heterodimeric interleukin receptors). Preferably, the bi- or multivalent peptide units bound to the carrier unit bind the same receptor target. However, they can of course still differ in their amino acid sequence. The monomeric binding units of the bi- or multivalent peptide units can be either linear or cyclic. A cyclic molecule can be for example created by the formation of intramolecular cysteine bridges.

The polymeric carrier unit comprises at least one natural or synthetic branched, linear or dendritic polymer. The polymeric carrier unit is preferably soluble in water and body fluids and is preferably a pharmaceutically acceptable polymer. Water soluble polymer moieties include, but are not limited to, e.g. polyalkylene glycol and derivatives thereof, including PEG, PEG homopolymers, mPEG, polypropyleneglycol homopolymers, copolymers of ethylene glycol with propylene glycol, wherein said homopolymers and copolymers are unsubstituted or substituted at one end e.g. with an acylgroup; polygiycerines or polysialic acid; carbohydrates, polysaccharides, cellulose and cellulose derivatives, including methylcellulose and carboxymethylcellulose; starches (e.g. hydroxyalkyl starch (HAS), especially hydroxyethyl starch (HES) and dextrines, and derivatives thereof; dextran and dextran derivatives, including dextransulfat, crosslinked dextrin, and carboxymethyl dextrin; chitosan (a linear polysaccharide), heparin and fragments of heparin; polyvinyl alcohol and polyvinyl ethyl ethers; polyvinylpyrrollidon; alpha,beta-poly[(2-hydroxyethyl)-DL-aspartamide; and polyoxyethylated polyols. One example of a carrier unit is a homobifunctional polymer, of for example polyethylene glycol (bis-maleimide, bis-carboxy, bis-amino etc.).

The polymeric carrier unit which is coupled to the peptide units according to the present invention can have a wide range of molecular weight due to the different nature of the different polymers that are suitable in conjunction with the present invention. There are thus no size restrictions. However, it is preferred that the molecular weight is at least 3 kD, preferably at least 10 kD and approximately around 20 to 500 kD and more preferably around 30 to 150 or around 60 or 80 kD. The size of the carrier unit depends on the chosen polymer and can thus vary. For example, especially when starches such as hydroxyethylstarch are used, the molecular weight might be considerably higher. The average molecular weight might then be arranged around 100 to 4,000 kD or even be higher. However, it is preferred that the molecular weight of the HES molecule is about 100 to 300 kD, preferably around 200 kD. The size of the carrier unit is preferably chosen such that each peptide unit is respectively can be optimally arranged for binding their respective receptor molecules.

In order to facilitate this, one embodiment of the present invention uses a carrier unit comprising a branching unit. According to this embodiment, the polymers, as for example PEG, are attached to a branching unit thus resulting in a large carrier molecule allowing the incorporation of numerous peptide units. Examples for appropriate branching units are glycerol or polyglycerol. Also dendritic branching units can be used as for example taught by Haag 2000, herein incorporated by reference. Also the HES carrier may be used in a branched form. This e.g. if it is obtained to a high proportion from amylopectin.

Preferably after the peptide units are created by combining the monomeric binding units (either head to head, head to tail, or tail to tail) to peptide units the polymeric carrier unit is connected/coupled to the peptide units. The polymeric carrier unit is connected to the peptide units via a covalent or a non-Covalent (e.g. a coordinative) bond. However, the use of a covalent bond is preferred.

The attachment can occur e.g., via a reactive amino acid of the peptide units e.g. lysine, cysteine, histidine, arginine, aspartic acid, glutamic acid, serine, threonine, tyrosine or the N-terminal amino group and the C-terminal carboxylic acid. In case the peptide does not carry a respective reactive amino acid, such an amino acid can be introduced into the amino acid sequence. The coupling should be chosen such that the binding to the target is not or at least as little as possible hindered. Depending on the conformation of the peptide unit the reactive amino acid is either at the beginning, the end or within the peptide sequence.

In case the polymeric carrier unit does not possess an appropriate coupling group, several coupling substances/linker can be used in order to appropriately modify the polymer in order that it can react with at least one reactive group on the peptide unit to form the supravalent compound. Suitable chemical groups that can be used to modify the polymer are e.g. as follows:

Acylating groups which react with the amino groups of the protein, for example acid anhydride groups, N-acylimidazole groups, azide groups, N-carboxy anhydride groups, diketene groups, dialkyl pyrocarbonate groups, imidoester groups, and carbodiimide-activated carboxyl-groups. All of the above groups are known to react with amino groups on proteins/peptides to form covalent bonds, involving acyl or similar linkages;

alkylating groups which react with sulfhydryl (mercapto), thiomethyl, imidazo or amino groups on the peptide unit, such as halo-carboxyl groups, maleimide groups, activated vinyl groups, ethylenimine groups, aryl halide groups, 2-hydroxy 5-nitro-benzyl bromide groups; and aliphatic aldehyde and ketone groups together with reducing agents, reacting with the amino group of the peptide;
ester and amide forming groups which react with a carboxyl group of the peptide, such as diazocarboxylate groups, and carbodiimide and amine groups together;
disulfide forming groups which react with the sulfhydryl groups on the protein, such as 5,5′-dithiobis (2-nitrobenzoate) groups, ortho-pyridyl disulfides and alkylmercaptan groups (which react with the sulfhydryl groups of the peptide in the presence of oxidizing agents such as iodine);
dicarbonyl groups, such as cyclohexandione groups, and other 1,2-diketone groups which react with the guanidine moieties of protein;
diazo groups, which react with phenolic groups on the peptide;
reactive groups from reaction of cyanogens bromide with the polysaccharide, which react with amino groups on the protein.

Thus in summary, the compound according to the invention may be made by—optionally—first modifying the polymeric carrier chemically to produce a polymeric carrier having at least one chemical group thereon which is capable of reacting with an available or introduced chemical group on the peptide unit, and then reacting together the—optionally—modified polymer and the peptide unit to form a covalently bonded complex thereof utilising the chemical group of the—if necessary—modified polymer.

In case coupling occurs via a free SH-group of the peptide, the use of a male imide group in the polymer is preferred.

In order to generate a defined molecule it is preferred to use a targeted approach for attaching the peptide units to the polymeric carrier unit. In case no appropriate amino acids are present at the desired attachment site, appropriate amino acids should be incorporated in the peptide unit. For site specific polymer attachment a unique reactive group e.g. a specific amino acid at the end of the peptide unit is preferred in order to avoid uncontrolled coupling reactions throughout the peptide leading to a heterogeneous mixture comprising a population of several different polymeric molecules.

The coupling of the peptide units to the polymeric carrier unit, e.g. PEG or HES, is performed using reactions principally known to the person skilled in the art. E.g. there are number of PEG and HES attachment methods available to those skilled in the art (see for example WO 2004/100997 giving further references, Roberts et al., 2002; U.S. Pat. No. 4,064,118; EP 1 398 322; EP 1 398 327; EP 1 398 328; WO 2004/024761; all herein incorporated by reference).

It is important to understand that the concept of supravalency described herein is different from the known concept of PEGylation or HESylation. In the state of the art e.g. PEGylation is only used in order to produce either peptide dimers or in order to improve pharmacokinetic parameters by attaching one or more PEG units to a peptide. However, as outlined above, the attachment of two or more at least bivalent peptide units to e.g. HES as a polymeric carrier unit also greatly enhances efficacy (thus decreasing the EC50-dose). The concept of this invention thus has strong effects on pharmacodynamic parameters and not only on pharmacokinetic parameters as it is the case with the PEGylation or HESylation concepts known in the state of the art. However, of course the incorporation of for example PEG or HES as polymeric carrier unit also has the known advantages regarding pharmacokinetics.

PEGylation is usually undertaken to improve the biopharmaceutical properties of the peptides. The most relevant alterations of the protein molecule following PEG conjugation are size enlargement, protein surface and glycosylation function masking, charge modification and epitope shielding. In particular, size enlargement slows down kidney ultrafiltration and promotes the accumulation into permeable tissues by the passive enhance permeation and retention mechanism. Protein shielding reduces proteolysis and immune system recognition, which are important routes of elimination. The specific effect of PEGylation on protein physicochemical and biological properties is strictly determined by protein and polymer properties as well as by the adopted PEGylation strategy.

However, the use of PEG or other non-biodegradable polymers might lead to new problems.

During in vivo applications, dosage intervals in a clinical setting are triggered by loss of effect of the drug. Routine dosages and dosage intervals are adapted such that the effect is not lost during dosage intervals. Due to the fact that peptides attached to a non-biodegradable, large polymer unit (e.g. a PEG-moiety) can be degraded faster than the support molecule can be eliminated by the body, a risk of accumulation of the carrier unit can arise. Such a risk of accumulation always occurs as effect-half life time of the drug is shorter than elimination half life time of the drug itself or one of its components/metabolites. Thus, accumulation of the carrier molecule should be avoided especially in long-term treatments because peptides are usually PEGylated with very large PEG-moieties (˜20-40 kD) which thus show a slow renal elimination. The peptide moiety itself undergoes enzymatic degradation and even partial cleavage might suffice to deactivate the peptide.

In order to find a solution to this potential problem one embodiment of the present invention teaches the use of a polymeric carrier unit that is composed of at least two subunits. The polymeric subunits are connected via biodegradable covalent linker structures. According to this embodiment the molecular weight of the large carrier molecule (for example 40 kD) is created by several small or intermediate sized subunits (for example each subunit having a molecular weight of 5 to 10 kD), that are connected via biodegradable linkers. The molecular weights of the modular subunits add up thereby generating the desired molecular weight of the carrier molecule. However, the biodegradable linker structures can be broken up in the body thereby releasing the smaller carrier subunits (e.g. 5 to 10 kD). The small carrier subunits show a better renal clearance than a polymer molecule having the overall molecular weight (e.g. 40 kD). An illustrating example is given in FIG. 16.

The linker structures are selected according to known degradation properties and time scales of degradation in body fluids. The breakable structures can, for instance, contain cleavable groups like carboxylic acid derivatives as amide/peptide bonds or esters which can be cleaved by hydrolysis (see e.g. Roberts, 2002 herein incorporated by reference). PEG succinimidyl esters can also be synthesized with various ester link-ages in the PEG backbone to control the degradation rate at physiological pH (Zhao, 1997, herein incorporated by reference). Other breakable structures like disulfides of benzyl urethanes can be cleaved under mild reducing environments, such as in endosomal compartments of a cell (Zalipsky, 1999) and are thus also suitable. Other criteria for selection of appropriate linkers are the selection for fast (frequently enzymatic) degradation or slow (frequently non-enzymatic decomposition) degradation. Combination of these two mechanisms in body fluids is also feasible. It is clear that this highly advantageous concept is not limited to the specific peptide units described or referred to herein but also applies to other pharmaceutical molecules that are attached to large polymer units such as PEG molecules wherein the same problems of accumulation arises.

According to one embodiment hydroxyalkyl starch and preferably HES is used as polymeric carrier unit. HES has several important advantages. First of all, HES is biodegradable. Furthermore, the biodegradability of HES can be controlled via the ratio of hydroxyethyl groups and can thus be influenced. A molar degree of substitution of 0.4-0.8 (in average 40-80% of the glucose units contain a hydroxyethyl group) are well suitable for the purpose of the present invention. Due to the biodegradability, accumulation problems as described above in conjunction with PEG do usually not occur. Furthermore, HES has been used for a long time in medical treatment e.g. in form of a plasma expander. Its innocuousness is thus approved.

Furthermore, derivatives of hydrolysis products of HES are detectable by gas chromatography. HES-peptide conjugates can be hydrolysed under conditions under which the peptide units are still stable. This allows the quantification and monitoring of the degradation products and allows evaluations and standardisations of the active peptides.

According to a further embodiment a first type of polymeric carrier unit is used and loaded with peptide units. This first carrier is preferably easily biodegradable as is e.g. HES. However, not all attachment spots of the first carrier are occupied with peptide units but only e.g. around 20 to 50%. Depending on the size of the used polymer, several hundred peptide units can be coupled to the carrier molecule. However, depending on the peptide used usually less peptide units (2 to 50, 2 to 20, 2 to 10 or 3 to 5) are coupled. The rest (or at least some) of the remaining attachment spots are occupied with a different carrier, e.g. small PEG units having a lower molecular weight than the first carrier. This embodiment has the advantage that a supravalent composition is created due to the first carrier which is however, very durable due to the presence of the second carrier, which is constituted preferably by PEG units of 3 to 5 or to 10 kD. However, the whole entity is very well degradable, since the first carrier (e.g. HES) and the peptide units are biodegradable and the second carrier, e.g. PEG is small enough to be easily cleared from the body.

The monomers constituting the binding domains of the peptide units recognize a binding site of a target. The term binding domain refers to the binding part of the monomeric peptide that is involved in binding the target. Depending on the peptide, the binding domain may be composed of different structural motives of the peptide (e.g. beta-sheets, alpha-helices, beta turns) that define the binding domain in the three dimensional conformation of the peptide.

According to one embodiment, the peptide unit binds to a transmembrane receptor. Activation of transmembrane receptors by growth factors and cytokines generally occurs when a ligand binds to a specific domain on the receptor, thereby inducing a conformational change and/or triggering dimerization or oligomerization of receptor chains. Upon ligand binding, several members of the class I cytokine receptors form homodimers, including the erythropoietin receptor (EPOR), thrombopoietin receptor (TPOR), granulocyte colony-stimulating factor receptor (G-CSFR), growth hormone receptor (GHR), and prolactin receptor (PrR). These class I cytokine receptors show structural and functional similarities among each other. According to one embodiment the peptide units are chosen such that they bind to these class I cytokine receptors.

As outlined, a homodimeric receptor is any biological target protein being composed by two non-covalently associated identical protein subunits. Such receptors usually are only functional if both subunits are associated in the homodimeric form. The latter property of being a homodimeric receptor differentiates the EPO-Receptor and e.g. the related TPO-receptor from many other cytokine receptors. In most other cases of cytokine receptors, the receptor is a heterodimer (many interleukin-receptors) or even a heterotrimer (e.g. IL-2).

The peptide units according to the invention, comprising at least two monomeric binding domains bind their target and preferably are able to di-respectively multimerise the target and/or stabilize it accordingly thereby creating a signal transduction inducing complex. The peptide units have preferably a homodimeric target molecule, which is preferably a cytokine receptor (see above).

As outlined above, the peptide units used for creating the supravalent molecules bind the target receptor. According to one embodiment, the peptide units act as receptor agonists. The term agonist refers to a biologically active peptide which binds to its complementary biologically active receptor (target) and activates the latter either to cause a biological response in the receptor or to enhance preexisting biological activity of the receptor (target). According to a different embodiment, the peptide unit acts as a receptor antagonist. An antagonist also binds to its complementary biologically active receptor (target). However, an antagonist does not induce or enhance the biological activity of the receptor (target).

Several methods are known in the state of the art for dimerising or multimerising monomeric peptide units. These methods can be used for creating the peptide units according to the invention. The prevailing solutions following the dimerization approach are characterized by
a) the fact, that the binding domains are first synthesized separately as monovalent or monomeric peptides, which can be modified e.g. by attachment of reactive groups in preparation for step b
b) in a second reaction step, two—in most cases identical—binding domains are joined together in separate dimerization reaction, which can also include linker molecules usually being interposed between the two dimerized domains.

Such dimers are examples of bivalent (dimeric) peptides and exhibit essentially the same biological functions as the monomers but show enhanced biological activity due to a better interaction with the receptor.

Several techniques are known to the person skilled in the art to dimerize or oligomerize the monomers which can also be applied according to the teachings of the present invention. Monomers can be dimerized e.g. by covalent attachment to a linker. A linker is a joining molecule creating a covalent bond between the polypeptide units of the present invention. The polypeptide units can be combined via a linker in such a way, that the binding to the EPO receptor is improved (Johnson et al. 1997; Wrighton et al. 1997). It is furthermore referred to the multimerization of monomeric biotinylated peptides by non-covalent interaction with a protein carrier molecule described by Wrighton et al (Wrighton, 1997). It is also possible to use a biotin/streptavidin system i.e. biotinylating the C-terminus of the peptides and a subsequent incubating the biotinylated peptides with streptavidin. Alternatively, it is known to achieve dimerization by forming a diketopiperazine structure. This method known to the skilled person is described in detail e.g. in Cavelier et al. (in: Peptides: The wave of the Future; Michal Lebl and Richard A. Houghten (eds); American Peptide Society, 2001). The disclosure of these documents regarding the dimerization and a non-covalent multimerization is incorporated herein by reference. Another alternative way to obtain peptide dimers known from prior art is to use bifunctional activated dicarboxylic acid derivatives as reactive precursors of the later linker moieties, which react with N-terminal amino groups, thereby forming the final dimeric peptide (Johnson et al, 1997). Monomers can also be dimerized by covalent attachment to a linker. Preferably the linker comprises NH—R—NH wherein R is a lower alkylene substituted with a functional group such as carboxyl group or amino group that enables binding to another molecule moiety. The linker might contain a lysine residue or lysine amide. Also PEG may be used a linker. The linker can be a molecule containing two carboxylic acids and optionally substituted at one or more atoms with a functional group such as an amine capable of being bound to one or more PEG molecules. A detailed description of possible steps for oligomerization and dimerization of peptides with a linking moiety is also given in WO 2004/101606.

Though being functionally sufficient and thus usable according to the teachings of the present invention, the prior art approaches of synthesizing dimeric molecules might have some disadvantages for some peptides.

One potential drawback could be perceived in the fact that the monomers to be connected have first to be synthesized separately. Because of the stochastic pairing of monomeric peptides during the dimerization reaction, it is in particular difficult to (selectively and intentionally) obtain heterodimeric bivalent peptides with this approach. At least this would lead to great losses in yield of a special, intended heterodimer. Bivalent peptides harboring two or more slightly different monomeric binding domains are very desirable, since due to their heterodimeric nature, special interactions between the two domains, which are able to stabilize their interaction in the final bivalent peptide, can be introduced while maintaining or even increasing binding to the homodimeric receptor. However, due to the high losses in yield associated with the prior art “stochastic dimerization reactions”, this is usually economically not an attractive approach.

Applying the prior art approaches for dimerization—even though technically suitable—have thus some economic disadvantages for providing these peptides with heterogeneous binding domains as described. Thus preferably a more efficient strategy is used to obtain highly active bivalent peptide units, which even might contain heterogenous binding domains.

The core concept of this dimerization method is to refrain from synthesizing the monomeric peptides forming part of the bivalent peptide in separate reactions prior dimerization or multimerization, but to synthesize the final peptide unit having at least two binding domains in one step as a single peptide; e.g. in one single solid phase reaction. Thus a separate dimerization or multimerization step as taught by the state of the art is no longer needed. This aspect provides a big advantage, i.e. the complete and independent control on each sequence position in the final peptide unit. The method allows to easily harbor at least two different receptor-specific binding domains in a peptide unit due to independent control on each sequence position.

According to this embodiment the sequence of the final peptide between the binding domains (which is the “linker region”) is composed of amino acids only, thus leading to one single, continuous peptide unit. In a preferred embodiment of the invention the linker is composed of natural or unnatural amino acids which allow a high conformational flexibility. In this regard it can be advantageous to use glycine residues as linking amino acids, which are known for their high flexibility in terms of torsions. However, also other amino acids, such as alanine or beta-alanine, or a mixture thereof can be used. The number and choice of used amino acids depend on the respective steric facts. This embodiment allows the custom-made design of a suitable linker by molecular modeling in order to avoid distortions of the bioactive conformation and thus allows perfect matching with the receptor units. A linker composed of 3 to 5 amino acids is especially preferred.

It is noteworthy that the linker between the functional binding domains (also referred to as monomeric units) of the peptide units can be either a distinct part of the peptide or can be composed—fully or in parts—of amino acids which are part of the monomeric binding domains. Thus the term “linker” is rather defined functionally than structurally, since an amino acid might form part of the linker unit as well as of the monomeric binding subunits.

Since—as mentioned above—during the synthesis of the bivalent peptide each sequence position within the final peptide is under control and thus can be precisely determined it is possible to custom- or tailor make the peptide units or specific regions or domains thereof, including the linker. This is of specific advantage since it allows the creation of specific attachment sites for the polymer and the avoidance of distortion of the bioactive conformation of peptide units due to unfavorable intramolecular interactions. The risk of distortions can be assessed prior synthesis by molecular modeling. This especially applies to the design of the linker between the monomeric domains.

The continuous bivalent/multivalent peptides according to the invention show much higher activity then the corresponding monomeric peptides and thus confirm the observation known from other dimeric peptides that an increase of efficacy is associated with bivalent peptide concepts.

According to a preferred embodiment, all peptide units (wherein each peptide unit is considered as one receptor binding unit) bind the same target receptor. However, they can be heterogeneous thus differing in their amino acid sequence.

According to one preferred embodiment said peptide units bind the EPO receptor thereby dimerising the EPO receptor complex. Preferably they induce signal transduction and are thus EPO receptor agonists. The peptide units respectively the monomeric binding domains creating the peptide units can be selected from the group of EPO mimetic peptides. Appropriate EPO mimetic peptides are well-known in the state of the art and can be used in connection with the present invention (please refer e.g. to WO 96/40772; WO 96/40749; WO 01/38342; WO 01/091780; WO 2004/101611; WO 2004/100997; WO 2004/101600; WO 2004/101606).

Further suitable EPO mimetic peptide units that can be used according to the present invention comprise binding domains of at least 10 amino acids in length that bind to the EPO receptor. They do preferably not comprise proline in the position commonly referred to as position 10 of the EPO mimetic peptide (for the numbering please refer to Wrighton et. al, 1996 and Johnson, 1998), but a positively charged amino acid. These EPO mimetic peptide carry an amino add motif characteristic for a beta-turn structure (Wrighton et al.), wherein the binding domain of the peptide unit according to this embodiment does not comprise a praline in said beta-turn motif at the position 10 but a positively charged amino acid, preferably either K or R. Also other basic amino acids, especially unnatural amino acids such as homoarginine might be used. The positions 9 and 10 of the EPO mimetic binding domain can be occupied by 5-aminolevulinic acid (5-Als). The peptide domain can also carry a R in position 17.

According to one embodiment at least one of the EPO receptor binding domains of the peptide units comprises the following sequence of amino acids:
X₆X₇X₈X₉X₁₀X₁₁X₁₂X₁₃X₁₄X₁₅
   wherein each amino acid is selected from natural or unnatural amino acids and
   X₆ is C, A, E, α-amino-γ-bromobutyric acid or homocysteine (hoc);

### X₇ is R, H, L, W or Y or S;

X₈ is M, F, I, homoserinmethylether (ham) or norisoleucine;
X₉ is G or a conservative exchange of G;
X₁₀ is a non conservative exchange of praline;
or X₉ and X₁₀ are substituted by a single amino acid:
X₁₁ is independently selected from any amino acid;

### X₁₂ is T or A;

X₁₃ is W, 1-nal, 2-nal, A or F;

### X₁₄ is D, E, I, L or V;

X₁₅ is C, A, K, α-amino-γ-bromobutyric acid or homocysteine (hoc)
provided that either X₆ or X₁₅ is C or hoc.

The length of one binding domain of said peptide unit is preferably between ten to forty or 50 or 60 amino acids. In preferred embodiments, the peptide consensus depicts a length of at least 10, 15, 18 or 20 amino acids. Of course they can be embedded respectively be comprised by longer sequences. The described monomeric peptide sequences can be perceived as binding domains for the EPO receptor. As EPO mimetic peptides they are capable of binding to and activating the EPO receptor.

It was very surprising, that these peptides do exhibit EPO mimetic activities although one or—according to some embodiments—even both prolines may be replaced by other natural or non-natural amino acids. In fact the peptides according to the invention have an activity comparable to that of proline-containing peptides. However, it is noteworthy that the amino acids substituting proline residues do not represent a conservative exchange but instead a non-conservative exchange. Preferably, a positively charged amino acid such as basic amino acids such as K, R and H and especially K is used for substitution. The non-conservative amino acid used for substitution can also be a non-natural amino acid and is preferably one with a positively charged side chain. Also comprised are respective analogues of the mentioned amino acids. A suitable example of a non-natural amino acid is homoarginine. According to one embodiment the peptide carries a positively charged amino acid in position 10 except for the natural amino acid arginine. According to this embodiment the praline 10 is thus substituted by an amino acid selected from K, H or a non-natural positively charged amino acid such as e.g. homoarginine. It is preferred that the peptides depict a lysine or homoarginine in position 10. As described above, also the proline in position 17 might be replaced by a non-conservative amino acid. In this respect it is also preferred, that said non-conservative amino acid is one with a positively charged side chain such as K, R, H or a respective non-natural amino acid such as e.g. homoarginine. According to a sub-embodiment of this embodiment the peptide carries a positively charged amino acid in position 17 except for the natural amino acid arginine. According to this embodiment the proline 17 is thus substituted by an amino acid selected from K, H or a non-natural positively charged amino acid such as homoarginine. It is preferred that the peptides depict a lysine or homoarginine in position 17.

The EPO-R binding domain can furthermore comprise a sequence of the following amino acids:
X₆X₇X₈X₉X₁₀X₁₁X₁₂X₁₃X₁₄X₁₅
   wherein each amino acid is indicated by standard letter abbreviation and

### X₆ is C;

### X₇ is R, H, L or W;

### X₈ is M, F or I;

X₉ is G or a conservative exchange of G;
X₁₀ is a non conservative exchange of proline;
X₁₁ is independently selected from any amino acid;

### X₁₂ is T;

### X₁₃ is W;

### X₁₄ is D, E, I, L or V;

### X₁₅ is C.

Furthermore, X₇ can be serine, X₈ can be hsm or norisoleucine and X₁₃ can also be 1-nal, 2-nal, A or F. The length of the peptide consensus is preferably between ten to forty or fifty or sixty amino acids. In preferred embodiments, the peptide consensus comprises at least 10, 15, 18 or 20 amino acids.

Further EPO mimetic peptides that can be used in order to create the peptide units according to the present invention are defined by the following peptide consensus sequences:

A peptide being capable of binding the EPO receptor, selected from the group consisting of

peptides comprising the following consensus sequence of amino acids:
X₆X₇X₈X₉X₁₀X₁₁X₁₂X₁₃X₁₄X₁₅
   wherein each amino acid is selected from natural or unnatural amino acids and
   X₆ is an amino acid with a sidechain functionality capable of forming a covalent bond or A or α-amino-γ-bromobutyric acid;

### X₇ is R, H, L, W, Y or S;

X₈ is M, F, I, homoserinemethylether or norisoleucine;
X₉ is G or a conservative exchange of G;
X₁₀ is a non conservative exchange of proline (or according to another embodiment proline or a conservative exchange of proline);
or X₉ and X₁₀ are substituted by a single amino acid;
X₁₁ is selected from any amino acid;
X₁₂ is an uncharged polar amino acid or A;
X₁₃ is W, 1-nal, 2-nal, A or F;

### X₁₄ is D, E, I, L or V;

X₁₅ is an amino acid with a sidechain functionality capable of forming a covalent bond or A or α-amino-γ-bromobutyric acid and

functionally equivalent fragments, derivatives and variants of the peptides defined by the above consensus sequence, that depict an EPO mimetic activity and have an amino acid in position X₁₀ that constitutes a non-conservative exchange of proline (or according to another embodiment proline or a conservative exchange of proline) or wherein X₉ and X₁₀ are substituted by a single amino acid.

According to the consensus sequence of the first embodiment, X₆ and X₁₅ depict amino acids with a sidechain functionality capable of forming a covalent bond. These amino acids are thus capable of forming a bridge unit. According to one embodiment, the amino acids in position X₆ and X₁₅ are chosen such that they are capable of forming an intramolecular bridge within the peptide by forming a covalent bond between each other. Forming of an intramolecular bridge may lead to cyclisation of the peptide. Examples for suitable bridge units are the disulfide bridge and the diselenide bridge. Suitable examples of amino acids depicting such bridge forming functionalities in their side chains are e.g. cysteine and cysteine derivatives such as homocysteine or selenocysteine but also thiolysine. The formation of a diselenide bridge e.g. between two selenocysteine residues even has advantages over a cysteine bridge. This as a selenide bridge is more stable in reducing environments. The conformation of the peptide is thus preserved even under difficult conditions.

However, it is evident that also amino acids are suitable in position X₆ and X₁₅, depicting a side chain with a functionality allowing the formation of different covalent bonds such as e.g. an amide bond between an amino acid having a positively charged side chain (e.g. the proteinogenic amino acids K, H, R or ornithine, DAP or DAB) and an amino acid having a negatively charged side chain (e.g. the proteinogenic amino acids or E). Further examples are amide and thioether bridges.

A peptide of at least 10 amino acids in length, capable of binding to the EPO receptor selected from the following alternatives

(a) a peptide comprising the following core sequence of amino acids:
X₉X₁₀X₁₁X₁₂X₁₃
   wherein each amino acid is selected from natural or non-natural amino acids, and wherein:
   X₉ is G or a conservative exchange of G;
   X₁₀ is a non conservative exchange of proline (or according to another embodiment praline or a conservative exchange of proline); or X₉ and X₁₀ are substituted by a single amino acid;
   X₁₁ is selected from any amino acid;
   X₁₂ is an uncharged polar amino acid or A;
   X₁₃ is naphthylalanine.
   (b) a peptide being capable of binding the EPO receptor comprising the following sequence of amino acids:
X₆X₇X₈X₉X₁₀X₁₁X₁₂X₁₃X₁₄X₁₅
   wherein each amino acid is selected from natural or unnatural amino acids and
   X₆ is C, A, E, α-amino-γ-bromobutyric acid or homocysteine (hoc);

### X₇ is R, H, L, W or Y or R, H, L, W, Y or S;

X₈ is M, F, I, homoserinemethylether or norisoleucine;
X₉ is G or a conservative exchange of G;
X₁₀ is a non conservative exchange of praline;
or X₉ and X₁₀ are substituted by a single amino acid;
X₁₁ is selected from any amino acid;

### X₁₂ is T or A;

X₁₃ is 1-nal, 2-nal

### X₁₄ is D, E, I, L or V;

X₁₅ is C, A, K, α-amino-γ-bromobutyric acid or homocysteine (hoc) provided that either X₅ or X₁₅ is C or hoc
(c) functionally equivalent fragments, derivatives and variants of the peptides defined by the above consensus sequences that depict an EPO mimetic activity and have an amino acid in position X₁₀ that constitutes a non-conservative exchange of proline (or according to another embodiment praline or a conservative exchange of praline) or wherein X₉ and X₁₀ are substituted by a single amino acid and a naphthylalanine in position X₁₃.

A peptide of at least 10 amino acids in length, capable of binding to the EPO receptor and comprising an agonist activity, selected from the group consisting of

peptides comprising at least one of the following core sequences of amino acids:
X₉X₁₀X₁₁X₁₂X₁₃;
X₉X₁₀X₁₁X₁₂X₁₃X₁₄X₁₅X₁₆X₁₇ or
X₉X₁₀X₁₁X₁₂X₁₃X₁₄X₁₅X₁₆X₁₇X₁₈X₁₉
   wherein each amino acid is selected from natural or non-natural amino acids and wherein in at least one of the positions X₁₀, X₁₇ or X₁₉ is a negatively charged amino acid and wherein
   X₉ is G or a conservative exchange of G;
   X₁₁ is selected from any amino acid;
   X₁₂ is an uncharged polar amino acid or A; preferably threonine, serine, asparagine or glutamine;
   X₁₃ is W, 1-nal, 2-nal, A or F;

### X₁₄ is D, E, I, L or V;

X₁₅ is an amino acid with a sidechain functionality capable of forming a covalent bond or A or α-amino-γ-bromobutyric acid;
X₁₆ is independently selected from any amino acid, preferably G, K, L, Q, R, S, Har or T;
X₁₈ is independently selected from any amino acid, preferably L or Q;

functionally equivalent fragments, derivatives and variants of the peptides defined by the above consensus sequences, that depict an EPO mimetic activity and wherein in at least one of the positions X₁₀, X₁₇ or X₁₉ is a negatively charged amino acid.

The EPO mimetic peptide having a negatively charged amino acid in at least one of the positions X₁₀, X₁₇ or X₁₉ may also be described by the following enlarged consensus sequence
X₆X₇X₈X₉X₁₀X₁₁X₁₂X₁₃X₁₄X₁₅X₁₆X₁₇X₁₈X₁₉
wherein each amino acid is selected from natural or non-natural amino acids and wherein
X₆ is an amino acid with a sidechain functionality capable of forming a covalent bond or A or α-amino-γ-bromobutyric acid;
X₇ is R, H, L, W or Y or S;
X₈ is M, F, I, Y, H, homoserinemethylether or norisoleucine;
X₉ is G or a conservative exchange of G;
in case X₁₀ is not a negatively charged amino acid, X₁₀ is praline, a conservative exchange of praline or a non conservative exchange of praline or
X₉ and X₁₀ are substituted by a single amino acid;
X₁₁ is selected from any amino acid;
X₁₂ is an uncharged polar amino acid or A; preferably threonine, serine, asparagine or glutamine;
X₁₃ is W, 1-nal, 2-nal, A or F;
X₁₄ is D, E, I, L or V;
X₁₅ is an amino acid with a sidechain functionality capable of forming a covalent bond or A or α-amino-γ-bromobutyric acid;
X₁₆ is independently selected from any amino acid, preferably G, K, L, Q, R, S, Her or T;
in case X₁₇ is not a negatively charged amino acid, X₁₇ is selected from any amino acid, preferably A, G, P, Y or a positively charged natural, non-natural or derivatized amino acid, preferably K, R, H, ornithine or homoarginine;
X₁₈ is independently selected from any amino acid, preferably L or Q;
in case X₁₉ is not a negatively charged amino acid, X₁₉ is independently selected from any amino acid, preferably a positively charged amino acid such as K, R, H, ornithine or homoarginine;
provided that at least one of X₁₀, X₁₇ or X₁₉ is a negatively charged amino acid.

A peptide of at least 10 amino acids in length, capable of binding to the EPO receptor and comprising an agonist activity, selected from the following group of peptides:

(a) a peptide, comprising the following core sequence of amino acids:
X₉X₁₀X₁₁X₁₂X₁₃;
X₉X₁₀X₁₁X₁₂X₁₃X₁₄X₁₅X₁₆X₁₇ or
X₉X₁₀X₁₁X₁₂X₁₃X₁₄X₁₅X₁₆X₁₇X₁₈X₁₉
wherein each amino acid is selected from natural or non-natural amino acids, and wherein:
X₉ is G or a conservative exchange of G;
X₁₁ is selected from any amino acid;
X₁₂ is an uncharged polar amino acid or A; preferably threonine, serine, asparagine or glutamine;
X₁₃ is W, naphthylalanine, A or F;
X₁₄ is D, E, I, L or V;
X₁₅ is an amino acid with a sidechain functionality capable of forming a covalent bond or A or α-amino-γ-bromobutyric acid,
as well as functionally equivalent fragments, derivatives and variants of the peptides defined by the above consensus sequence, that depict an EPO mimetic activity,
wherein at least one of the positions X₁₀, X₁₆, X₁₇ or X₁₉ depicts a positively charged non-proteinogenic amino acid having a side chain which is elongated compared to lysine;
   (b) a peptide, especially one being capable of binding the EPO receptor comprising the following sequence of amino acids:
X₆X₇X₈X₉X₁₀X₁₁X₁₂X₁₃X₁₄X₁₅
wherein each amino acid is selected from natural or unnatural amino acids and
X₆ is C, A, E, α-amino-γ-bromobutyric acid or homocysteine (hoc);
X₇ is R, H, L, W or Y or S;
X₈ is M, F, I, homoserinemethylether or norisoleucine;
X₉ is G or a conservative exchange of G;
X₁₀ is Har
X₁₁ is selected from any amino acid;
X₁₂ is T or A;
X₁₃ is W, 1-nal, 2-nal, A or F;
X₁₄ is D, E, I, L or V;
X₁₅ is C, A, K, α-amino-γ-bromobutyric acid or homocysteine (hoc)
provided that either X₆ or X₁₅ is C or hoc;
   (c) a peptide, comprising the following amino acid sequence
X₃X₄X₅X₆X₇X₈X₉X₁₀X₁₁X₁₂X₁₃X₁₄X₁₅X₁₆X₁₇X₁₈
wherein X₆ to X₁₅ have the above meaning of variant (b) and wherein
X₃ is independently selected from any amino acid, preferably D, E, L, N, S, T or V;
X₄ is Y;
X₅ is independently selected from any amino acid, preferably A, H, K, L, M, S, T or I.
X₁₆ is independently selected from any amino acid, preferably G, K, L, Q, R, S or T;
X₁₇ is homoarginine;
X₁₈ is independently selected from any amino acid.

These peptides may also be described by the following care sequence of amino is acids:
X₆X₇X₈X₉X₁₀X₁₁X₁₂X₁₃X₁₄X₁₅X₁₆X₁₇X₁₈X₁₉
   wherein each amino acid is selected from natural or non-natural amino acids and wherein
   X₆ is an amino acid with a sidechain functionality capable of forming a covalent bond or A or α-amino-γ-bromobutyric acid;

### X₇ is R, H, L, W or Y or S;

X₈ is M, F, I, Y, H, homoserinemethylether or norisoleucine;
X₉ is G or a conservative exchange of G;
in case X₁₀ is not a positively charged non-proteinogenic amino acid having a side chain which is elongated compared to lysine, X₁₀ is proline, a conservative exchange of proline or a non conservative exchange of praline or X₉ and X₁₀ are substituted by a single amino acid;
X₁₁ is selected from any amino acid;
X₁₂ is an uncharged polar amino acid or A; preferably threonine, serine, asparagine or glutamine;
X₁₃ is W, 1-nal, 2-nal, A or F;

### X₁₄ is D, E, I, L or V;

X₁₅ is an amino acid with a sidechain functionality capable of forming a covalent bond or A or α-amino-γ-bromobutyric acid;
in case X₁₆ is not a positively charged non-proteinogenic amino acid having a side chain which is elongated compared to lysine, X₁₆ is independently selected from any amino acid, preferably G, K, L, Q, R, S or T;
in case X₁₇ is not a positively non-proteinogenic charged amino acid having a side chain which is elongated compared to lysine, X₁₇ is selected from any amino acid, preferably A, G, P, Y or a positively charged natural, non-natural or derivatized amino acid, preferably K, R, H or ornithine;
X₁₈ is independently selected from any amino acid, preferably L or Q;
in case X₁₉ is not a positively charged non-proteinogenic amino acid having a side chain which is elongated compared to lysine, X₁₉ is independently selected from any amino acid, preferably a positively charged amino acid such as K, R, H or ornithine; provided that at least one of X₁₀, X₁₆, X₁₇ or X₁₉ is a positively charged non-proteinogenic amino acid having a side chain which is elongated compared to lysine.

The monomeric EPO mimetic peptide units, at least two of which build up one peptide unit might comprise a single amino acid substituting the amino acid residues X₉ and X₁₀. Preferably both residues are substituted by one non-natural amino acid, e.g. 5-aminolevulinic acid or 5-aminovaleric acid.

In a further embodiment, the binding domains used in the peptide units comprise the following sequence
X₄X₅X₆X₇X₈X₉X₁₀X₁₁X₁₂X₁₃X₁₄X₁₅
   wherein X₆ to X₁₅ have the above meaning and wherein

### X₄ is Y;

X₅ is independently selected from any amino acid and is preferably A, H, K, L, M, 5, T or I.

The binding domains may be extended and may comprise the consensus sequence
X₃X₄X₅X₆X₇X₈X₉X₁₀X₁₁X₁₂X₁₃X₁₄X₁₅X₁₆X₁₇X₁₈
   wherein X₄ to X₁₅ have the above meaning and wherein
   X₃ is independently selected from any amino acid, preferably D, E, L, N, S, T or V;
   X₁₆ is independently selected from any amino acid, preferably G, K, L, Q, R, S or T, more preferred K, R, S or T;
   X₁₇ is independently selected from any amino acid, preferably A, G, P, R, K, Y or a non-natural amino acid with a positively charged side chain, more preferred R, K or an non-natural amino acid such as homoarginine,
   X₁₈ is independently selected from any amino acid.

In a further embodiment of the invention it is preferred that the peptides comprise X₆ as C, E, A or hoc, preferably C and/or X₇ as R, H or Y and/or X₈ as F or M and/or X₉ as G or A, preferably G and/or X₁₀ as K and/or X₁₁ as V, L, I, M, E, A, T or norisoleucine and/or X₁₂ as T and/or X₁₃ as W and/or X₁₄ as D or V and/or X₁₅ as C or hoc, preferably C and/or X₁₇ as P, Y or A. It is, however, also preferred that X₁₇ is K or a non-natural amino acid with a positively charged side chain such as homoarginine.

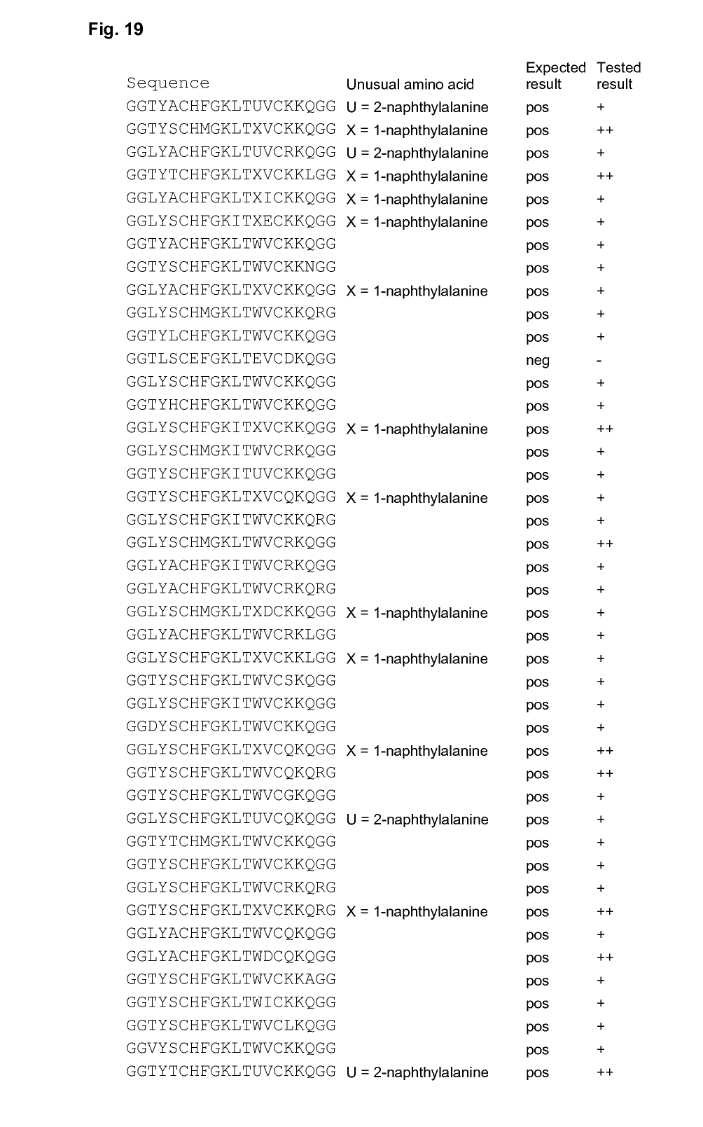

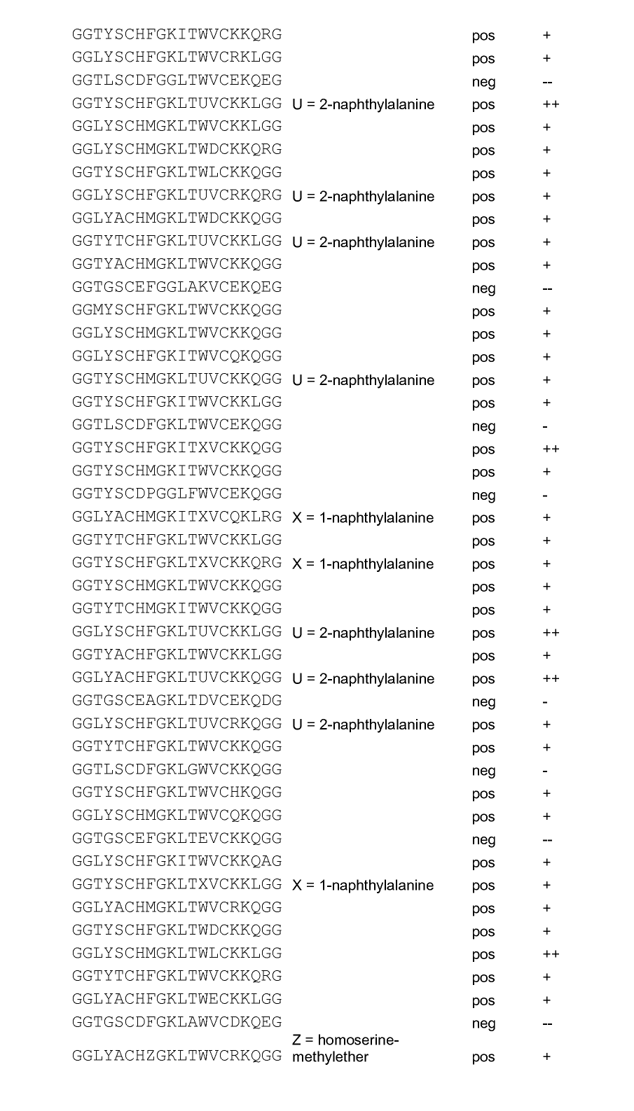

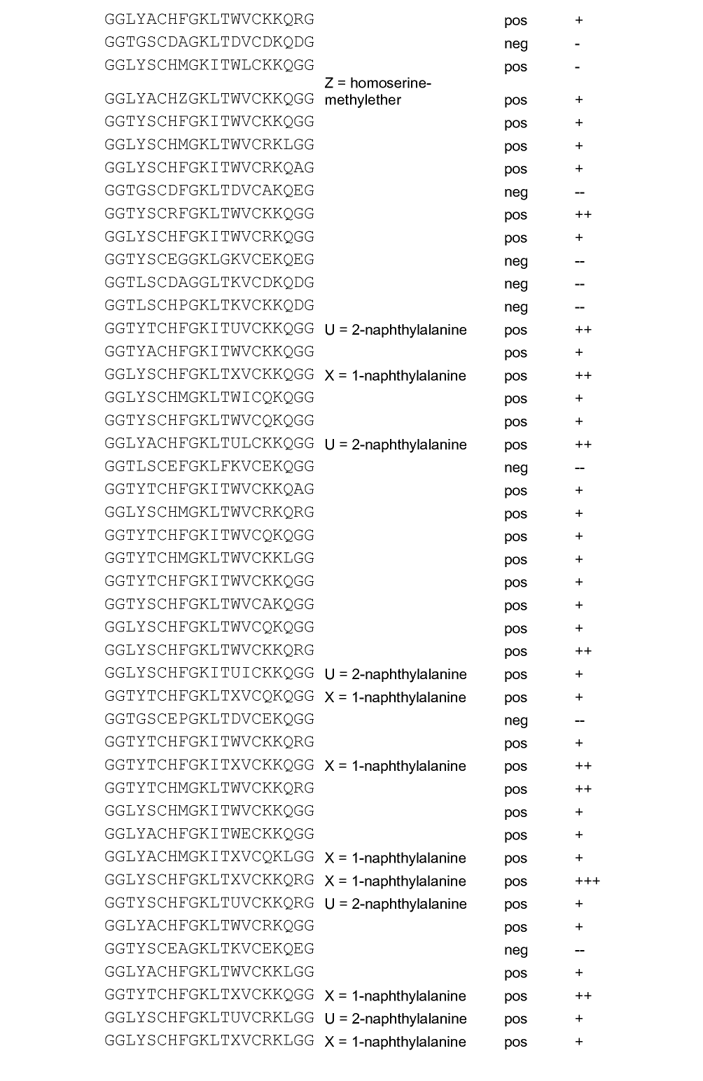

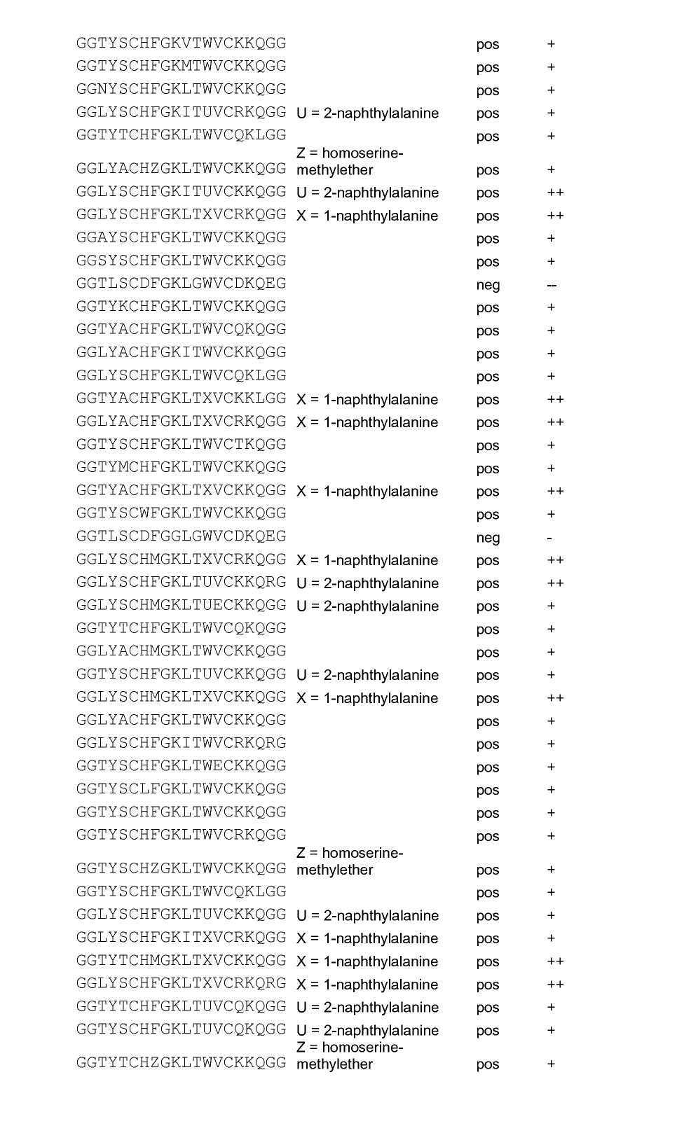

FIG. 19 discloses further novel and suitable peptide monomers depicting EPO mimetic activity. In conjunction with the present invention they can be used as suitable binding domains (monomers) for creating peptide units according to this invention. Furthermore, they can be used as monomeric or multimeric EPO mimetic peptides as described above.

At the beginning (N terminal) and end (C terminal) of the binding monomers, up to five amino acids may be removed and/or added.

As only the functional characteristics of the peptide are decisive—here the ability to bind to and in case of an EPO receptor agonist activate the EPO receptor. The precise amino acid sequence of the peptide unit may vary. Above are only given suitable examples for EPO mimetic peptides in order to support the general concept. However, also other EPO mimetic peptides with a differing amino acid sequence can be used in conjunction with the present invention.

According to another embodiment said peptide units bind the TPO receptor thereby dimerising the TPO receptor complex. Preferably they induce signal transduction and are thus TPO receptor agonists. The peptide units respectively the monomeric binding domains creating the peptide units can be selected from the group of TPO mimetic peptides. Appropriate TPO mimetic peptides are well-known in the state of the art and can be used in connection with the present invention. Suitable examples are e.g. described in WO 98/25965, U.S. Pat. No. 5,932,546, WO0024770, Cwirla, S. E., P. Balasubramanian, D. J. Duffin, C. R. Wagstrom, C, M. Gates, S. C. Singer, A. M. Davis, R. L. Tansik, L. C. Mattheakis, C. M. Boytos, P. J. Schatz, D. P. Baccanari, N. C. Wrighton, R. W. Barrett and W. J. Dower (1997). “Peptide agonist of the thrombopoietin receptor as potent as the natural cytokine.” Science 276(5319): 1696-1699, U.S. Pat. No. 6,083,913, U.S. Pat. No. 6,465,430, U.S. Pat. No. 5,869,451, U.S. Pat. No. 6,121,238, U.S. Pat. No. 6,251,864, Dower, W. J., S. E. Cwirla, P. Balasubramanian, P. J. Schatz, D. P. Baccanari and R. W. Barrett (1998). “Peptide agonists of the thrombopoietin receptor.” Stem Cells 16 Suppl 2: 21-29, WO05023834, WO0024770, the disclosure of all documents regarding the structurelamino acid sequence of the TP0 mimetic peptides is fully incorporated by reference.

The peptide units according to the invention may comprise besides L-amino acids or the stereoisomeric D-amino acids unnatural/unconventional amino acids, such as alpha,alpha-disubstituted amino acids, N-alkyl amino acids or lactic acid, e.g. 1-naphthylalanine, 2-naphthylalanine, homoserine-methylether, β-alanine, 3-pyridylalanine, 4-hydroxyproline, O-phosphoserine, N-methylglycine (sarcosine), N-acetylserine, N-acetylglycine, N-formylmethionine, 3-methylhistidine, 5-hydroxylysine, nor-lysine, 5-aminolevulinic acid or 5-aminovaleric acid. The use of N-methylglycine (MeG) and N-acetylglycine (AcG) is especially preferred, in particular in a terminal position. Also within the scope of the present invention are peptides which are retro, inverso and retro/inverso peptides of the defined peptides and those peptides consisting entirely of O-amino acids.

Also derivatives of the peptides may be used, e.g., oxidation products of methionine, or deamidated glutamine, arginine and C-terminus amide.

Herein, the abbreviations for the one-letter code as capital letters are those of the standard polypeptide nomenclature, extended by the addition of non-natural amino acids.

| Code | Amino acid |
|---|---|
| A | L-alanine |
| V | L-valine |
| L | L-leucine |
| I | L-isoleucine |
| M | L-methionine |
| F | L-phenylalanine |
| Y | L-tyrosine |
| W | L-tryptophan |
| H | L-histidine |
| S | L-serine |
| T | L-threonine |
| C | L-cysteine |
| N | L-asparagine |
| Q | L-glutamine |
| D | L-aspartic acid |
| E | L-glutamic acid |
| K | L-lysine |
| R | L-arginine |
| P | L-proline |
| G | glycine |
| Ava, 5-Ava | 5-aminovaleric acid |
| Als, 5-Als | 5-aminolevulinic acid |
| MeG | N-methylglycine |
| AcG | N-acetylglycine |
| Hsm | homoserine methylether |
| Har | homoarginine |
| 1nal | 1-naphthylalanine |
| 2nal | 2-naphthylalanine |
| βAla | beta-alanin |
| hoc/hcy | homocysteine |
| Ac | acetylated |
| Am | amidated |
| Dap | diamino propionic acid |
| Dab | diamino butyric acid |
| Aad | alpha-amino adipic acid |
| Asu | alpha-aminosuberic acid |
| Adi | adipic acid, |
| Glr | glutaric acid |
| Sec | selenocysteine |

The peptide units can be modified e.g. by conservative exchanges of single amino acids. Such an exchange alters the structure and function of a binding molecule but slightly in most cases. In a conservative exchange, one amino acid is replaced by another amino acid within a group with similar properties.

Examples of corresponding groups are:
amino acids having non-polar side chains: A, G, V, L, I, P, F, W, M
uncharged amino acids having polar side chains: 5, T, G, C, Y, N, Q
amino acids having aromatic side chains: F, Y, W
positively charged amino acids: K, R, H
negatively charged amino acids: D, E
amino acids of similar size or molecular weight, wherein the molecular weight of the replacing amino acids deviates by a maximum of +/−25% (or +/−20%, +/−15%, +/−10%) from the molecular weight of the original amino acid.

More specifically, Wrighton et al. (U.S. Pat. No. 5,773,569, and associated patents) examined in detail, using phage display techniques, which amino acids of an EPO-mimetic peptide can be replaced, while maintaining the activity. They also investigated and published data on possible truncation, i.e. minimal length of a given monomeric peptide.

According to one embodiment of the invention monomers selected from the group consisting of SEQ ID NOS 2, 4-9 given below are used for the formation of the at least bivalent peptide units. Good activity shows a peptide with K in position 10 and K in position 17 as in SEQ ID NO 2.

| | |
|---|---|
| SEQ ID NO 2: | GGTYSCHFGKLTWVCKKQGG |
| SEQ ID NO 4: | GGTYSCHFGKLTWVCKPQGG |
| SEQ ID NO 5: | GGTYSCHFGRLTWVCKPQGG |
| SEQ ID NO 6: | GGTYSCHFGRLTWVCKKQGG |

Incorporation of 5-aminolevulinic acid (Als):

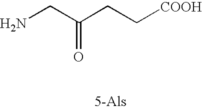

| | |
|---|---|
| SEQ ID NO 7: | GGTYSCHF-(Als)-LTWVCKPQGG |
| SEQ ID NO 8: | GGTYSCHF-(Als)-LTWVCKKQGG |
| SEQ ID NO 9: | GGTYSCHFGKLT-1nal-VCKKQRG |

According to one embodiment the peptide units are formed on the basis of the monomers according to SEQ ID NO 2 and 4 to 9 as given above or modifications thereof. The peptides can e.g. be modified by a conservative exchange of single amino acids, wherein preferably, not more than 1, 2 or 3 amino acids are exchanged. Preferably these peptides are modified as to AcG at the N-terminus and MeG at the C-terminus.

Some examples of appropriate peptide units for dimerising the EPO receptor are subsequently listed. The bars over the binding domains symbolize optional but preferred intramolecular disulfide bridges:

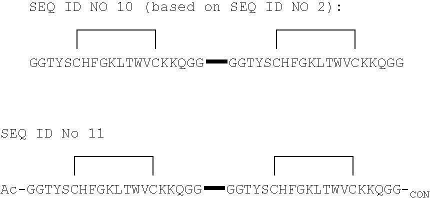

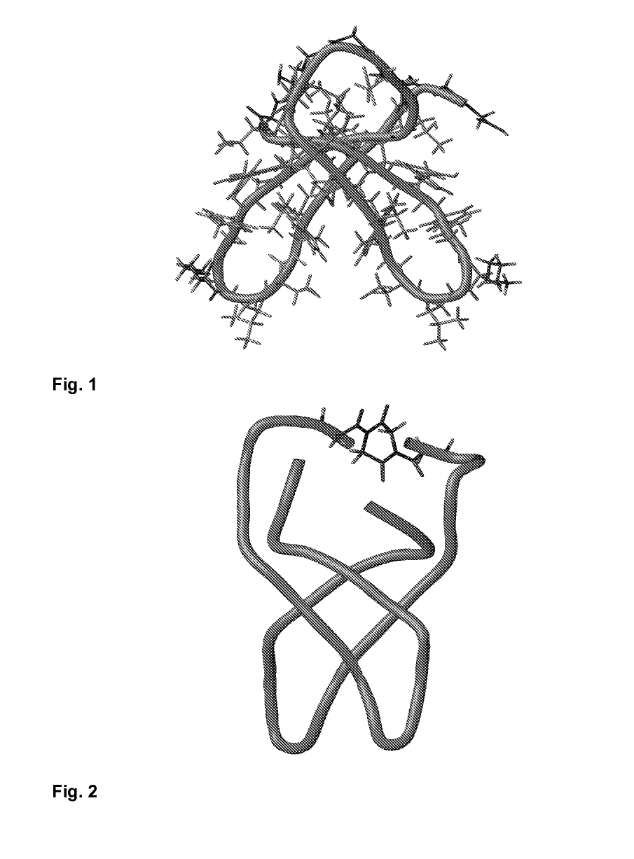

The linker in these bivalent structures is custom-made by molecular modelling to avoid distortions of the bioactive conformation (FIG. 1).

### SEQ ID NO 12

The linker sequence can be shortened by one glycine residue. This sequence is also an example for a linker composed by glycine residue forming at the same time part of the binding domain (see SEQ ID NO 2).

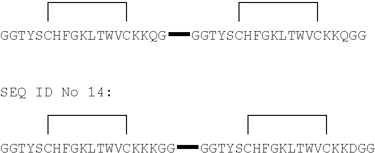

This sequence presents a continuous bivalent peptide unit harboring two slightly different (heterogeneous) binding domains. Such bivalent peptides would not be accessible economically with a prior art dimerization approach (see above). The advantage of this heterodimeric molecule lies therein that the deviating amino acids (presently K and D at the C-terminus) interact with each other thereby stabilizing the dimer. It is thus advantageous to incorporate respective stabilizing modifications in the molecule by molecular modeling.

A further example is

GGTYSCHFGKLT-1nal-VCKKQRG-GGTYSCHFGKLT-1nal-
VCKKQRG

According to a further embodiment the peptide optionally carries an additional amino acid, preferably one with a reactive side chain such as cysteine at the N-terminus such as e.g. in the following sequences

C-GGTYSCHFGKLTWVCKKQGG-GGTYSCHFGKLTWVCKKQGG
C-GGTYSCHFGKLT-1nal-VCKKQRG-GGTYSCHFGKLT-1nal-
VCKKQRG

The reactive side chain of cysteine may serve as a linking tie e.g. for attachment of the polymeric carrier unit. The peptides furthermore optionally comprise intramolecular disulfide bridges between the first and second and/or third and fourth cysteine.

The monomeric units as exemplified by SEQ ID 2, 4-9 and 12, 13 and 15, 15a can be favorably combined to the peptide units.

Examples for dimerization methods being applied to the monomers of the peptide units include (but are not limited to):
1. The dimerization via connection from C-terminus to C-terminus wherein the C-terminus of one of said monomeric peptide is covalently bound to the C-terminus of the other peptide. The linker/spacer between the monomers can contain a diketopiperazine unit. A preferred Gly-Gly diketopiperazine scaffold can be achieved by activating the C-terminal glycine monomer. This principle can also be use for forming a C-terminal dimerization.

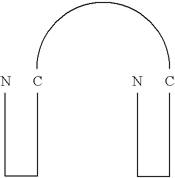

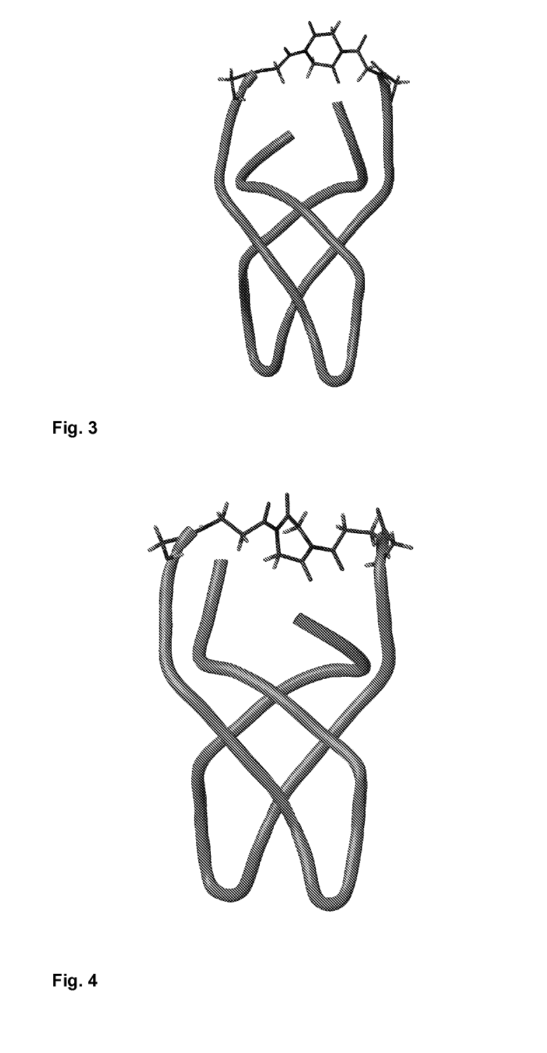

The following formulas and examples represent four customized examples which are optimized by molecular modeling:
   (a) dimer on the basis of SEQ ID NO 2 (the dimer conformation is showed in FIG. 2):

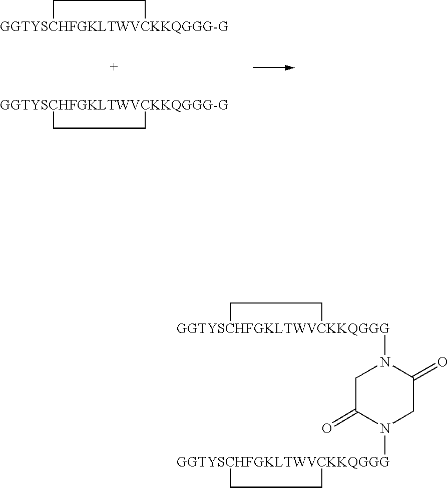

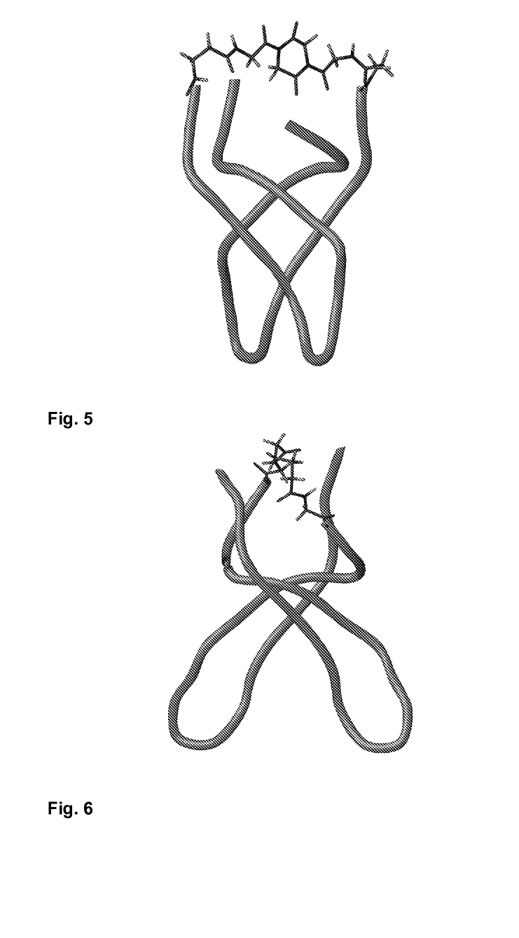

(b) dimer on the basis of SEQ ID NO 2 with a linker shortened by one glycine; the conformation is shown in FIG. 3.

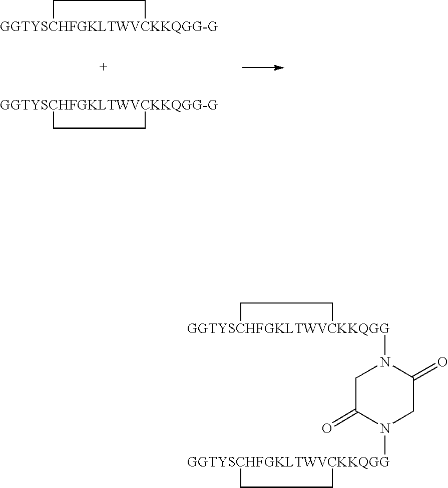

(c) dimer on the basis of SEQ ID NO 2 with a glycine substituted by beta-alanine (FIG. 4). The monomer (SEQ ID NO 16) is also applicable as EPO mimetic peptide.

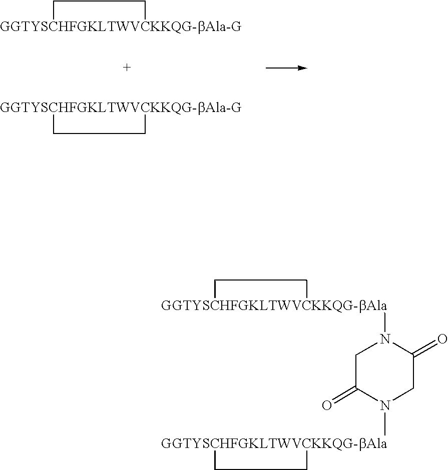

(d) dimer on the basis of SEQ ID NO 2 with an alternative glycine substituted by beta-alanine (FIG. 5). The monomer (SEQ ID NO 17) can also be applied as a EPO mimetic peptide.

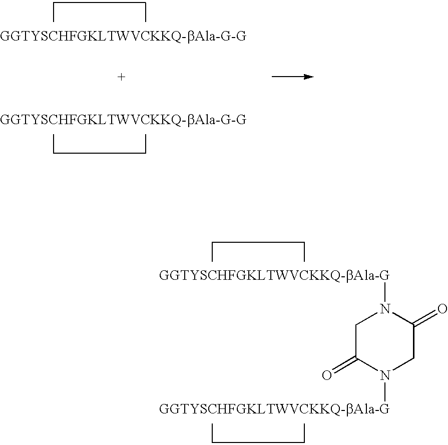

2. The dimerization via connection from N-terminus to N-terminus wherein the N-terminus of one of said monomeric peptides is covalently bound to the N-terminus of the other peptide, whereby the spacer unit is preferably containing a dicarboxylic acid building block.

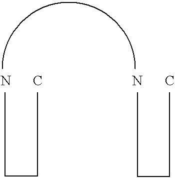

(a) In one embodiment the resulting dimers on the basis of SEQ ID NO 2 elongated at the N-Terminus by one glycine residue (SEQ ID NO 18) contain hexanedioyl unit as linker/spacer (FIG. 6):

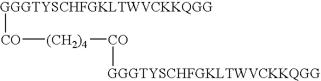

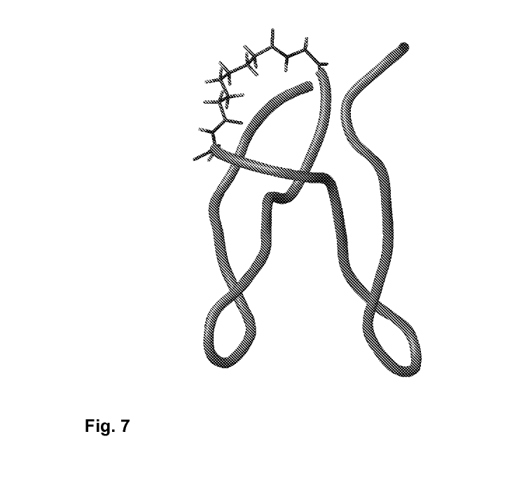

(b) in an alternative embodiment the dimerization can be achieved by using a octanedioyl unit as linker/spacer (FIG. 7):

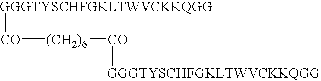

3. The dimerization via the side chains wherein an amino acid side chain of one of said monomeric peptides is covalently bound to an amino acid side chain of the other peptide with inclusion of a suitable spacer molecule connecting the two peptide monomers.

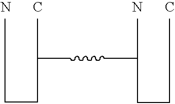

This can include:

(a) the connection via an amide bond.

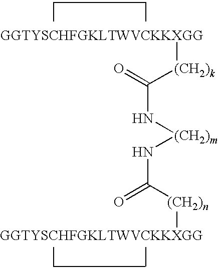

(b) or the connection via a disulfide bridge:

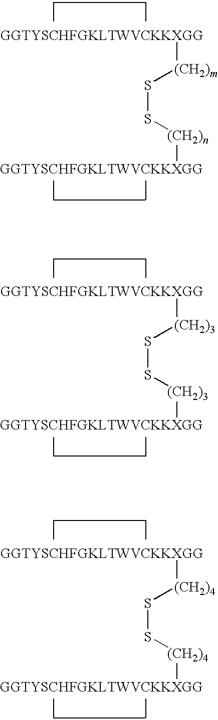

The X symbolizes the backbone core of the respective amino acid participating in the formation of the respective peptide bond.

According to a different strategy, the covalent bridge linking the peptide monomers to each other thereby forming the peptide unit is formed between the sidechains of the C-terminal amino acid of the first monomeric peptide unit and the N-terminal amino acid of the second peptide monomer. Hence, it is preferred according to this dimerization strategy that the monomeric peptides to be dimerized carry an amino acid with a bridge forming functionality at either the N- or C-terminus thereby allowing the formation of a covalent bond between the last amino acid of the first peptide and the first amino acid of the second peptide. The bond creating the dimer is preferably covalent. Suitable examples of respective bridges are e.g. the disulfide bridge and the diselenide bridge. However, also e.g. amide bonds between positively and negatively charged amino acids or other covalent linking bonds such as thioether bonds are suitable as linking moieties.

Preferred amino acids suitable for forming respective connecting bridges between the monomeric binding units to form the final peptide unit are e.g. cysteine, cysteine derivatives such as homocysteine or selenocysteine or thiolysine. They form either disulfide bridges or, in case of selenium containing amino acids, diselenide bridges.

Suitable examples for respectively created peptide unit dimers are given below using EPO mimetic peptides as examples:

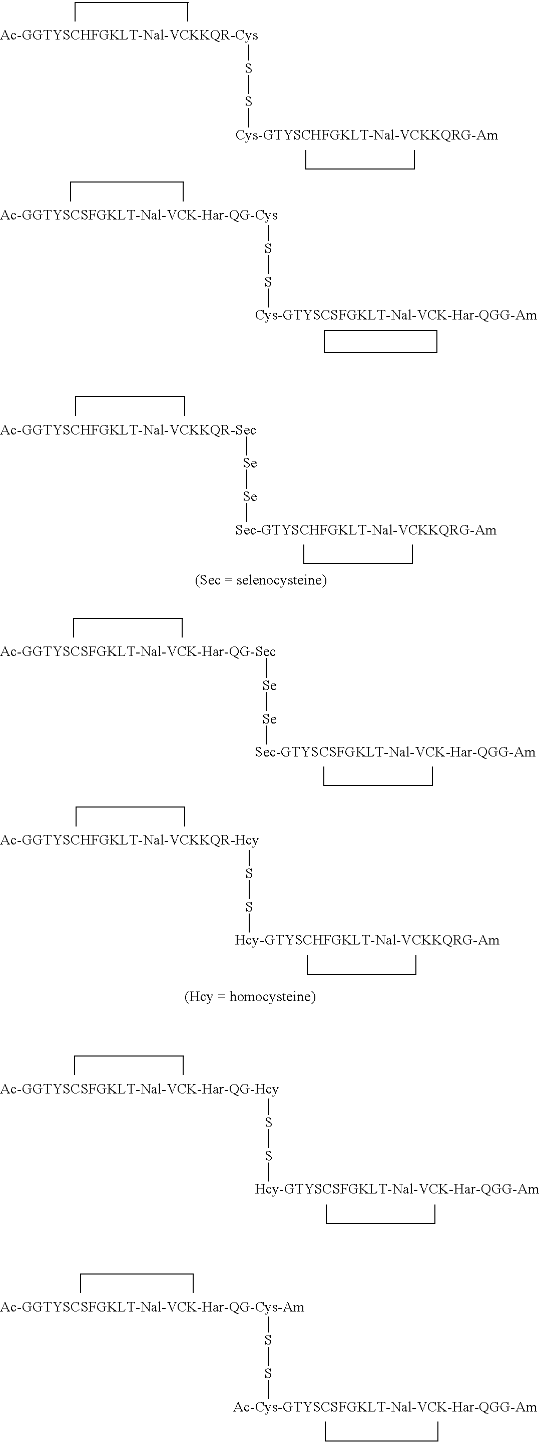

According to a further development either at the N- or the C-terminus of the peptide dimer (and hence of the respective monomeric peptide units either being located at the beginning or the end of the dimer) comprise an extra amino acid, allowing the coupling of the polymeric carrier such as HES in order to create the supravalent compound. Consequently, the introduced amino acid carries a respective coupling functionality such as e.g. an SH-group. One common example for such an amino acid is cysteine. However, also other amino acids with a functional group allowing the formation of a covalent bond (e.g. all negatively and positively charged amino acids) are suitable.

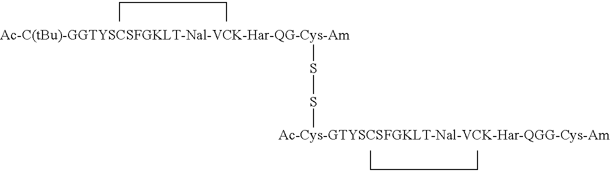

The bars over the peptide monomers represent covalent intramolecular bridges; in this case disulfide bridges.

According to a further development the amino acid at the C and/or the N terminus involved in forming the covalent bridge for connecting the monomeric units to a dimer depicts a charged group such as e.g. the COO⁻ or the NH₃⁺ group. This feature leads to a favourable stabilisation of the structure of the intermolecular bridge:

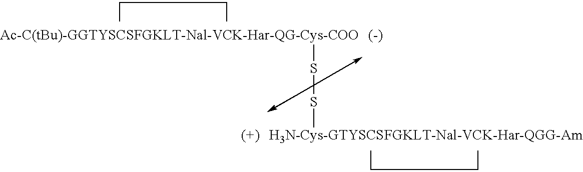

4. The dimerization by forming continuous bi- or multivalent peptides was already outlined above.

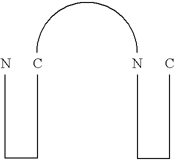

The core concept of this strategy refrains from synthesizing the monomeric peptides forming part of the multi- or bivalent peptide in separate reactions prior dimerization or multimerization, but to synthesize the final bi- or multivalent peptide in one step as a single peptide; e.g. in one single solid phase reaction. Thus a separate dimerization or multimerization step is no longer needed. This aspect provides a big advantage, i.e. the complete and independent control on each sequence position in the final peptide unit. The method allows to easily harbor at least two different receptor-specific binding domains in a peptide unit due to independent control on each sequence position.

As for the monomers and di- or multimeric peptides, the continuous bivalent/multivalent peptides can be modified by e.g. acetylation or amidation or be elongated at C-terminal or N-terminal positions.

All possible modifications also apply for modifying the linker. In particular it might be advantageous to attach soluble polymer moieties to the linker such as e.g. PEG, starch or dextrans.

The synthesis of the final multi- or bivalent peptide according to the invention favorably can also include two subsequent and independent formations of disulfide bonds or other intramolecular bonds within each of the binding domains. Thereby the peptides can also be cyclized.

The reactive side chains of the peptides may serve as a linking tie e.g. for further modifications. The peptide units furthermore optionally comprise intramolecular bridges between amino acids having a bridge forming side chain functionality such as e.g. the cysteines.

The peptides can be modified by e.g. acetylation or amidation or be elongated at the C-terminal or N-terminal positions. Extension with one or more amino acids at one of the two termini (N or C), e.g. for preparation of an attachment site for the polymeric carrier often leads to a heterodimeric bivalent peptide unit which can best be manufactured as a continuous peptide.

Several reactive amino acids are known in the state of the art in order to couple carriers to protein and peptides. A preferred coupling amino acid is cysteine which can be either coupled to the N or C terminus or be introduced within the peptide sequence. However, the coupling direction can make a considerable difference and should thus be carefully chosen for the peptide unit. This shall be demonstrated on the basis of the following example of an EPO mimetic peptide:

Used are the following two dimers:

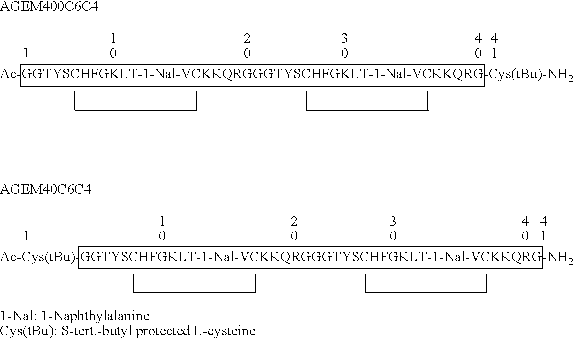

The 41mers AGEM400C6C4 and AGEM40C6C4 posses the same core sequence. The amino acids 1-40 of AGEM40C6C4 equal the amino acids 2-41 of AGEM40C6C4. The only difference is the position of the tBu-protected cysteine. This amino acid is not involved in the receptor drug interaction but is destined to function as the linking group to a polymeric carrier in the final conjugate. In case of AGEM400C6C4 the tBu-protected cysteine is attached to the C term, in case of AGEM40C6C4 it is attached to the N term. The connecting bars represent cysteine bridges.

There are two advantages of AGEM400C6C4 over AGEM40C6C4.

The first advantage is its synthetic accessibility. AGEM400C6C4 can be isolated in higher overall yields than AGEM40C6C4. In case of the synthesis of the linear sequence of AGEM40C6C4 a CIZ-22mer (C1Z-RGGGTYSCHFGKLT-1-NaI-VCKKQRG-NH₂, CIZ: 2-Chlorobenzyloxycarbonyl group) is observed as a byproduct. During purification of the linear sequence with reversed phase high pressure liquid chromatography (RP-HPLC) it exhibits a similar chromatographic behaviour as the linear precursor of AGEM40C6C4 and therefore makes it difficult to be separated from it leading to a loss in overall yield of the desired product. In case of AGEM400C6C4 no analogous compound is found.

The second advantage of AGEM400C6C4 over AGEM40C6C4 lies in the easier implementation of an analysis of the final conjugate of the deprotected peptide with a polymeric carrier. One strategy for the analysis of a peptide conjugate is the selective degradation of the conjugate by cleavage with endoproteases. Ideally the whole peptide is released from the polymeric carrier during the enzymatic hydrolysis. These peptide fragments can be identified and quantified by standard analytical techniques like i.e. HPLC with UV or MS detection, etc.

In case of AGEM400C6C4 the cleavage can be affected with trypsine—an endoprotease that is known to cleave highly selectively peptide bonds that lie C terminal of the charged amino acids arginine and lysine (F. Lottspeich, H. Zorbas (Hrsg.), “Bioanalytik”, Spectrum Akadernischer Verlag, Heidelberg, Berlin, 1998). Applied to conjugates of AGEM400C6C4 this will set free fragments that cover 38 of 41 amino acids of the original peptide bound to the carrier molecule. In case of AGEM40C6C4 fragments of only 21 of 41 amino acids are released by the tryptic digest:

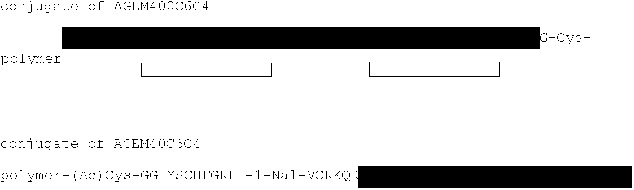

Fragments that are set free and can be detected by follow-up analyses are marked grey.

As the analysis of an Active Pharmaceutical Ingredient is a key issue during its development AGEM400C6C4 has a clear advantage over AGEM40C6C4.

Thus in case a positively charged amino acid is located in the respective positions, it is highly preferred to incorporate the linking amine acid (here cysteine) at the C-terminus because it possible to generate a nearly complete peptide fragment since a cleavage site is due to the arginine in position X₁₉ of the monomer pretty much right before the polymer.

Respective assembly methods as described above can also be used for the preparation of multimers as peptide units.

It is pointed out that all of the binding domains described herein either alone or as a part of a bivalent peptide can also be combined with one or more other either identical or different peptide domains in order to form respective homo- or heterogenous bi- or multivalent peptide units.

The peptide units are optionally modified as to AcG at the N-terminus and MeG at the C-terminus.

The peptide units can be modified by e.g. acetylation or amidation or be elongated at C-terminal or N-terminal positions. Extension with one or more amino acids at only one of the two termini, especially for preparation of later carrier unit attachment often leads to a heterodimeric bivalent peptide unit which can best be manufactured as a continuous peptide (see above).

The synthesis of the final multi- or bivalent peptide according to the invention favorably can also include two subsequent and independent formations of disulfide bonds or other intramolecular bonds within each of the binding domains.

The present invention further comprises respective compound production methods, wherein the peptide units are connected to the respective carrier units. The compounds of the present invention can advantageously be used for the preparation of human and/or veterinarian pharmaceutical compositions. The indications depend on the peptide units attached thereto.

In case of coupling of EPO mimetic peptides the compounds according to the present invention are especially suitable for the same indications as erythropoietin. Thus the present invention also provides a method for treating a patient suffering from a disorder that is susceptible to treatment with a erythropoietin agonist, comprising administering to the patient a therapeutically effective dose or amount of a compound of the present invention carrying a peptide unit comprising an erythropoietin agonist activity.

Erythropoietin is a member of the cytokine super family (see above). Besides the stimulating effects described in the introduction, it was also found that erythropoietin stimulates stem cells. The EPO mimetics described herein are thus suitable for all indications caused by stem cell associated effects. Non-limiting examples are the prevention and/or treatment of diseases associated with the nerve system. Examples are neurological injuries, diseases or disorders, such as e.g. Parkinsonism, Alzheimer's disease, Huntington's chorea, multiple sclerosis, amyotrophic lateral sclerosis, Gaucher's disease, Tay-Sachs disease, a neuropathy, peripheral nerve injury, a brain tumor, a brain injury, a spinal cord injury or a stroke injury. The EPO mimetic peptides according to the invention are also usable for the preventive and/or curative treatment of patients suffering from, or at risk of suffering from cardiac failure. Examples are cardiac infarction, coronary artery disease, myocarditis, chemotherapy treatment, alcoholism, cardiomyopathy, hypertension, valvar heart diseases including mitral insufficiency or aortic stenosis, and disorders of the thyroid gland, chronic and/or acute coronary syndrome. Furthermore, the EPO mimetics can be used for stimulation of the physiological mobilization, proliferation and differentiation of endothelial precursor cells, for stimulation of vasculogenesis, for the treatment of diseases related to a dysfunction of endothelial precursor cells and for the production of pharmaceutical compositions for the treatment of such diseases and pharmaceutical compositions comprising said peptides and other agents suitable for stimulation of endothelial precursor cells. Examples of such diseases are hypercholesterolaemia, diabetis mellitus, endothel-mediated chronic inflammation diseases, endotheliosis including reticulo-endotheliosis, atherosclerosis, coronary heart disease, myocardic ischemia, angina pectoris, age-related cardiovascular diseases, Raynaud disease, pregnancy induced hypertonia, chronic or acute renal failure, heart failure, wound healing and secondary diseases. The compounds carrying EPO mimetic peptide units are especially useful for the treatment of disorders that are characterized by a deficiency of erythropoietin or a low or defective red blood cell population and especially for the treatment of any type of anemia and stroke. Such pharmaceutical compositions may optionally comprise pharmaceutical acceptable carriers in order to adopt the composition for the intended administration procedure. Suitable delivery methods as well as carriers and additives are for example described in WO 2004/101611, herein incorporated by reference.

In case the compound carries TPO mimetic peptide units (having an agonist activity) the compounds may be used for all indications as thrombopoietin. They are thus useful for the prevention and treatment of diseases mediated by TPO, such as e.g. haematological disorders including thrombocytopenia, granulocytopenia and anemia, and the treatment of haematological malignancies. Thus the present invention also provides a method for treating a patient suffering from a disorder that is susceptible to treatment with a thrombopoietin agonist, comprising administering to the patient a therapeutically effective dose or amount of a compound of the present invention carrying a peptide unit comprising a thrombopoietin agonist activity.

### EXAMPLES

### A. Illustration of the Concept of Supravalent Compounds

The concept of the supravalent molecules shall be explained by means of examples. FIG. 13 shows an example of a simple supravalent molecule according to the invention. Two continuous bivalent peptides are connected N-terminally by a bifunctional PEG moiety carrying maleimide groups. Cysteine was chosen as reactive attachment site for the PEG carrier unit.

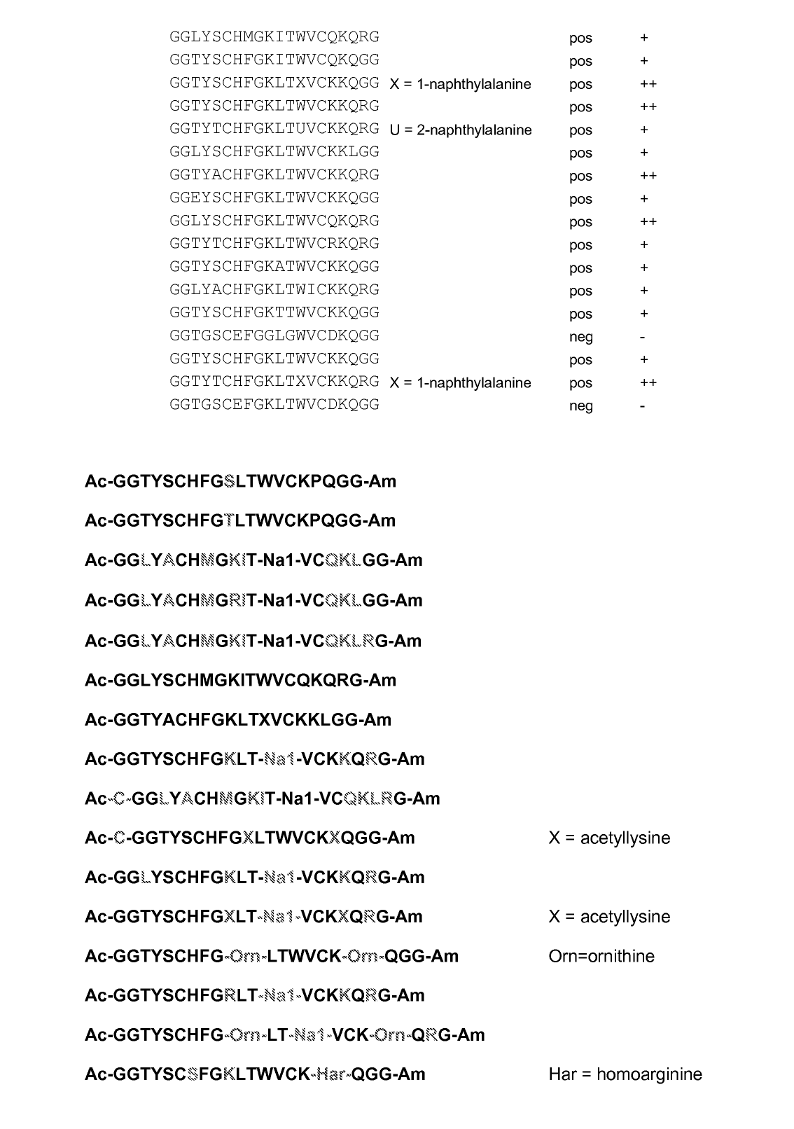

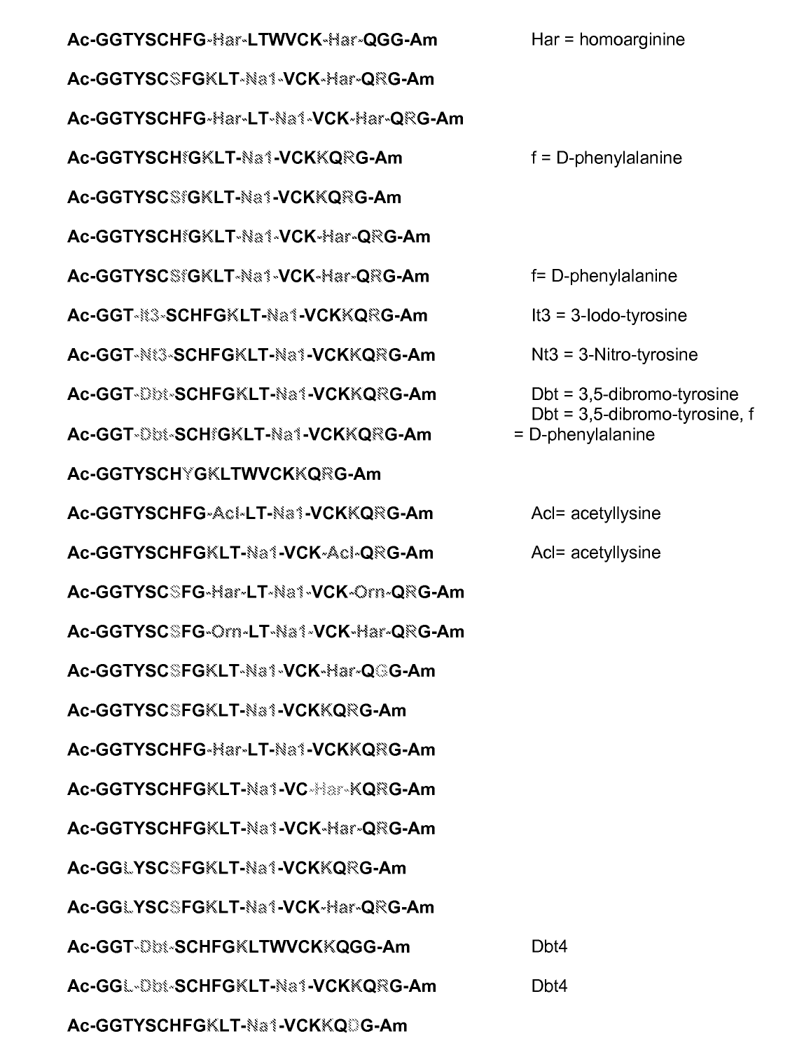

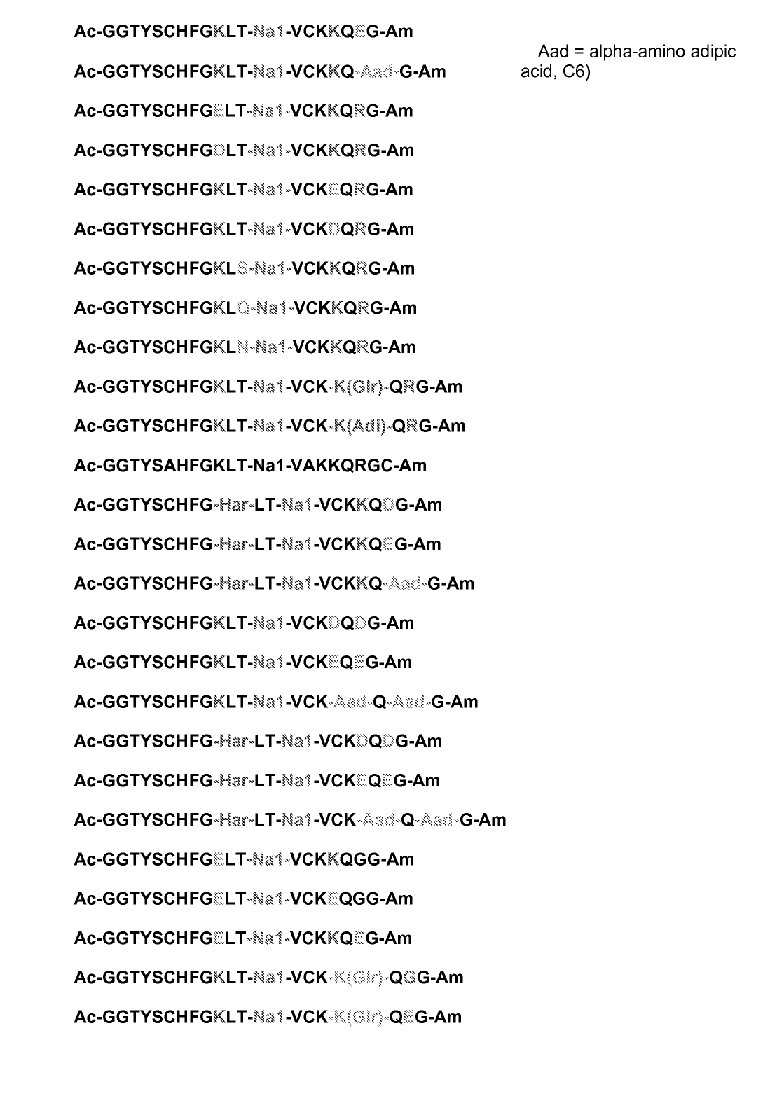

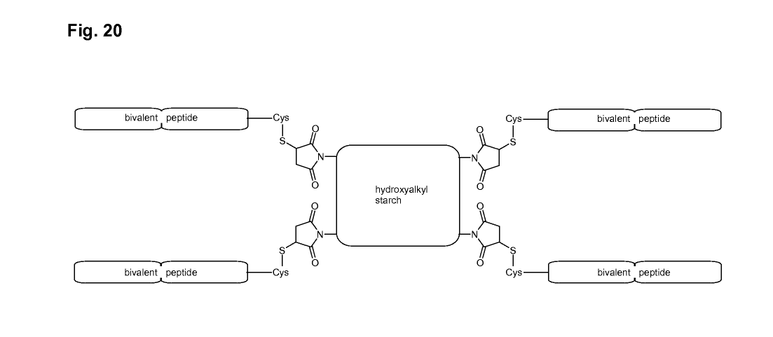

However, supravalent molecules can comprise more than two continuous bi- or multivalent peptide units. FIG. 14 gives an example that is based on a carrier unit with a central glycerol unit as branching unit and comprising three continuous bivalent peptides. Again cysteine was used for attachment. FIG. 20 shows an example using HES as polymeric carrier unit. HES was modified such that it carries maleimide groups reacting with the SH groups of the peptide units. According to the example, all attachment sites are bound to peptide units. However, also small PEG units (e.g. 3 to 10 kD) could occupy at least some of the attachment sites.

As explained above, the supravalent concept can also be extended to polyvalent dendritic polymers wherein a dendritic and/or polymer carrier unit is connected to a larger number of continuous bivalent peptides. For example, the dendritic branching unit can be, based on polyglycerol (please refer to Haag 2000, herein incorporated by reference).

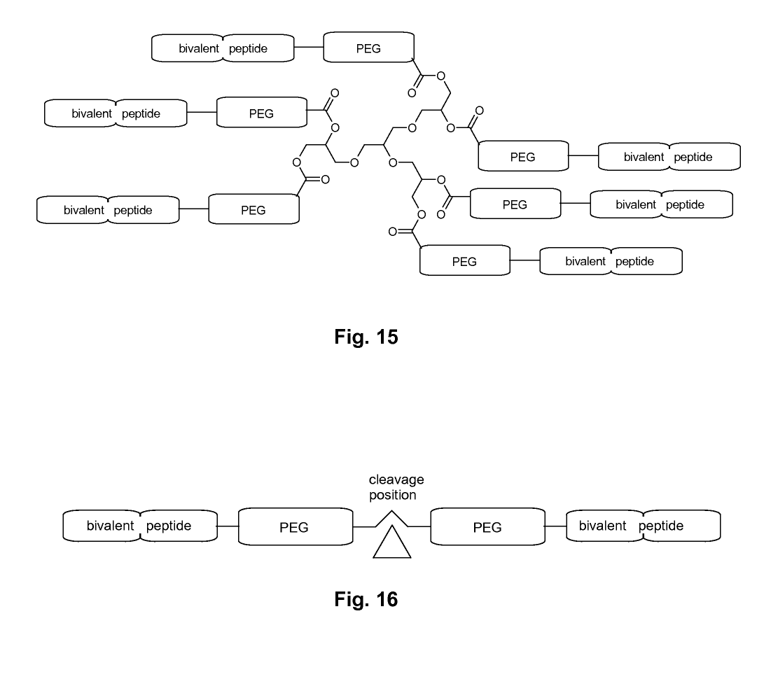

An example for a supravalent molecule based on a carrier unit with a dendritic branching unit containing six continuous bivalent peptides is shown in FIG. 15.

Other examples of supravalent molecules comprise carrier units with starches or dextrans, which are oxidized using e.g. periodic acid to harbor a large number of aldehyde functions. In a second step, many bivalent peptides are attached to the carrier unit and together form the final molecule. Please note that even several hundred (e.g. 50 to 1000, preferably 150 to 800, more preferably 250 to 700) peptide units can be coupled to the carrier molecule, which is e.g. HES. However, also far less peptide units may be bound to the HES molecule as it is shown in the Figs., especially if EPO mimetic peptides are coupled. The average number of peptide units to be coupled may be chosen from around 2 to 1000, 2 to 500, 2 to 100, 2 to 50, preferably 2 to 20 and most preferably 2 to 10, depending on the peptide and the receptor(s) to be bound.

FIG. 16 demonstrates the concept of a simple biodegradable supravalent molecule. Two continuous bivalent peptides are connected N-terminally by two bifunctional PEG moieties that are connected via a biodegradable linker having an intermediate cleavage position. The linkers allow the break up of the large PEG unit in the subunits thereby facilitating renal clearance.

The advantages connected to the supravalence effect were very surprising and unexpected. Initially it was feared, that the conjugation to a macromolecule might reduce efficacy. This expectation was based on the assumed disadvantages in binding rate due to reduced diffusion rates with larger molecules. Another expectation was, that from the several peptide APIs bound to a carrier not all would be able to bind to the receptor potentially due to sterical problems of simultaneous binding or because the number of receptors, which can be reached by the extensions of the macromolecular carrier is limited and possibly below the number of peptide APIs. Thus, an increase of potency of the peptide API (Active Pharmaceutical Ingredient) as is seen with the supravalence concept of the present invention was not expected.

On the other side, due to the significant pharmacokinetic changes a macromolecular carrier is able to introduce, the in vivo potency could have been improved due to the longer half life time of the whole peptide/carrier complex. This phenomenon also has the effect that a supravalence effect is difficult to determine in vivo, since it is a pharmacodynamic entity, which has to be determined separately. In vitro assays are thus not only sufficient, but might be the only useful way of clearly demonstrating the supravalence effect.

The supravalence effect as described in this invention can be demonstrated by comparison of molar amounts of peptide API (conjugated to a carrier vs. unconjugated).

An experiment was performed in a standard TF-1 cell assay as recommended by the European Pharmacopoe for the determination of EPO-like activity in vitro (please also see below). Basically, TF-1 cells (their proliferation being dependent from the presence of EPO-like activity) are cultured in the presence various concentrations of EPO or EPO-mimetic substances. The resulting cell numbers are quantified using colorimetric MTT-assay and photometric measurements. Based on these data, it is possible to determine normalized dose-response relations for each given substance.

In this assay EPO and the peptide AGEM40 (see below), the latter being a continuous bivalent peptide with known EPO-mimetic activity was used.

AGEM40 was used as unconjugated peptide and as peptide conjugated to macromolecular carrier (in this case hydroxyethylstarch of the mean molecular weight 130 kD; commercial source is the pharmacay, Voluven as plasma substitute). The Building Block Size of this Conjugate is roughly 40 kD, which means that the average HES-molecule carries about 2-5, preferably 3 to 4 peptide moieties. Also a HES 200/0.5 may be used. After modification of the 130 kD HES approximately 4 peptides of AGEM 40 were conjugated (molecular weight of the conjugated molecule: 150 kD). When a HES having a molecular weight of 200 kD was used, this amounts to approx. 5 peptide units conjugated to the HES (molecular weight of the conjugated molecule: 220 kD).

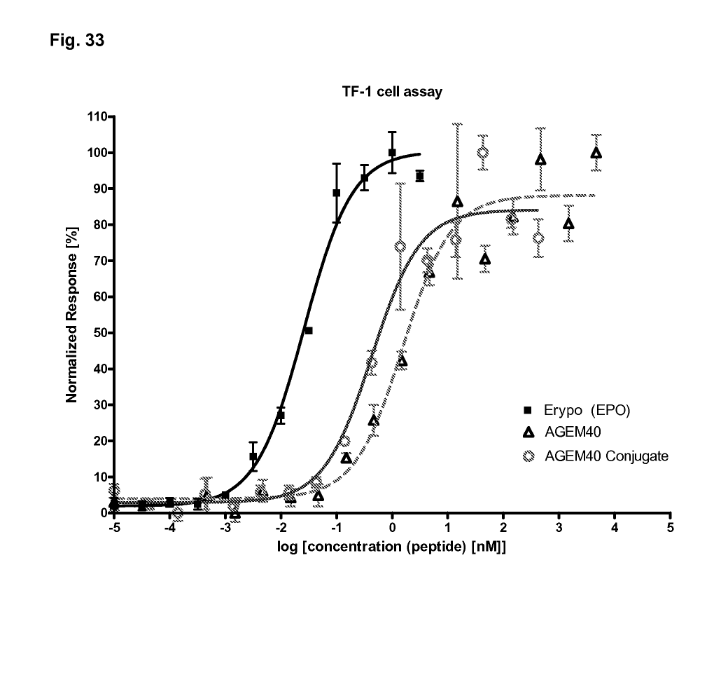

The comparison shown in FIG. 33 is based on molar comparison of peptide concentration, whether or not the peptide is conjugated. Surprisingly, potency is increasing (EC50 is decreasing and the dose response curve is situated left from the unconjugated peptide) thereby demonstrating a positive pharmacodynamic influence of oligovalent conjugation to a macromolecular carrier.

Thus—independent from the expected pharmacokinetic improvements—the conjugation concept according to the invention clearly increases potency of the overall active pharmaceutical ingredient (APi) and thus its efficacy.

This is a new mechanism, which can certainly be used for peptides addressing the EPO-receptor, but potentially also for other membrane bound pharmacological targets, especially other cytokine receptors such as those for thrombopoietin, G-CSF, Interleukins, and others (see above).

### B. Concepts for Conjugating Peptide Units to Hydroxyethyl Starch

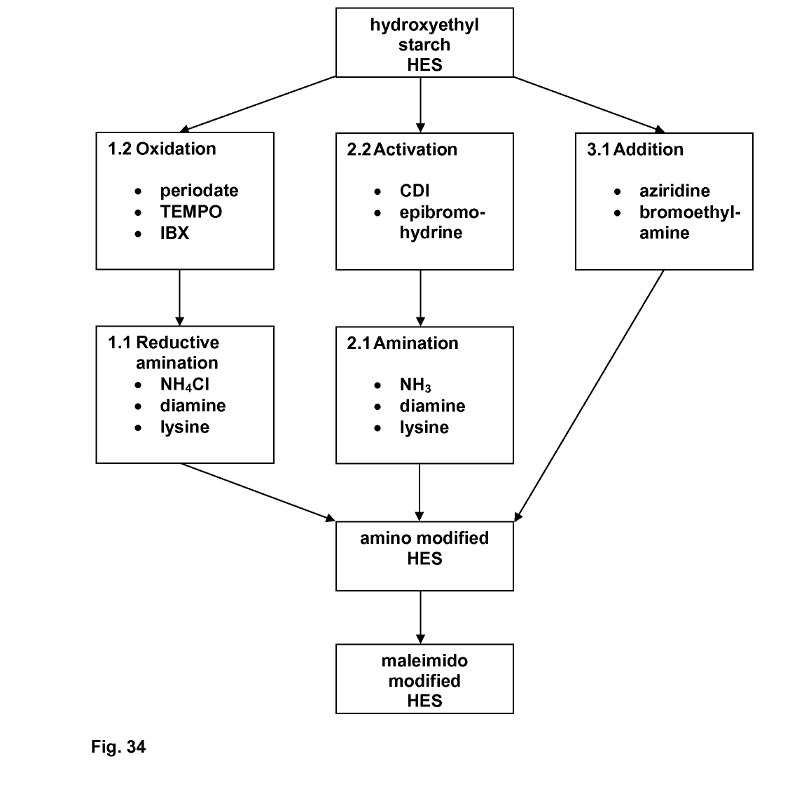

As outlined above, according to one embodiment, polysaccharides such as hydroxyalkyl starch and preferably hydroxyethyl starch is used as a polymeric carrier for the peptide units. In order to be able to conjugate the peptide units to the carrier, it is feasible to introduce appropriate linking groups into the starch molecule in order to facilitate coupling. According to one embodiment, amino groups are introduced onto the starch (hereinafter described upon the example hydroxyethyl starch) backbone. There are different strategies that can be followed. Three of them will be explained in more detail hereinafter, an overview over these methods is given in FIG. 34:

### 1. A Two-Step Process: Aldehyde Groups are Introduced by Oxidation and Followed by Reductive Amination.

The oxidation of the HES molecule can be accomplished by several oxidizing agents i.e. sodium periodate (NaIO₄), and 2-Iodoxybenzoic acid (IBX). The oxidation with NaIO₄ is long and well known and leads to aldehydes by opening of the saccharide rings.

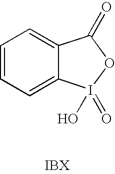

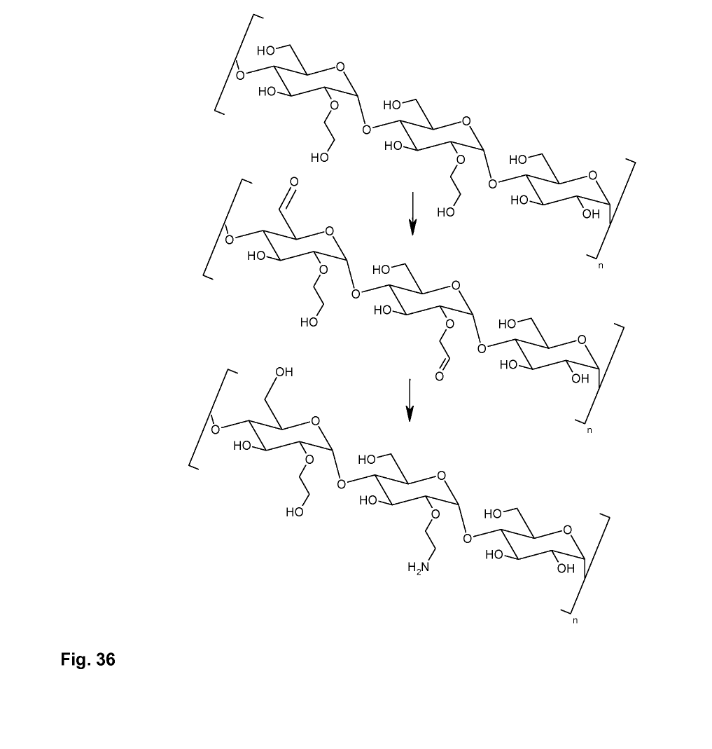

can be used stoichiometrically to convert primary alcohol groups to aldehydes without opening the saccharide rings (see FIG. 36, for review see: V. V. Zhdankin, Current Organic Synthesis, 2005, 2, 121-145 and cited papers). Derivatives that are better soluble in water are described in the literature (Thottumkara, A, P.; Vinod. T. K., Tetrahedron Lett., 2002, 43(4), 569).

According to a different approach, the carbohydrate (preferably a starch molecule such as HAS) is oxidized by contacting the starting material containing the carbohydrate (preferably a starch molecule such as HAS) with a reagent producing oxoammonium ion in the presence of an oxidizing agent or by contacting the starting material directly with the reactive species, the oxoammonium ion.

The oxidizing agent is e.g. a chemical oxidizing agent such as a hypohalide as e.g. sodium hypochlorite and hypobromite or hydrogen peroxide. Alternatively, an oxidative enzyme may be used as oxidizing agent (see e.g. WO 99/23240, herein incorporated by reference).

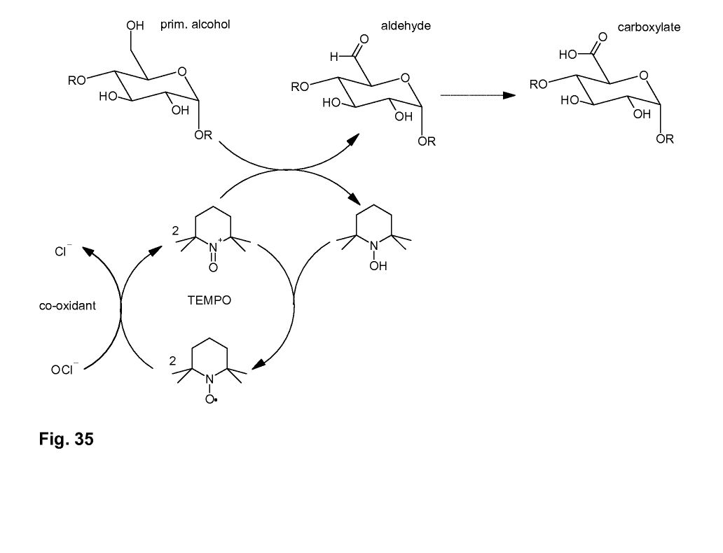

The reagent producing the oxoammonium ion is preferably a nitroxyl compound, more preferably a di-tert-nitroxyl compound such as 2,2,6,6-Tetramethylpiperidine-1-oxyl (TEMPO) or respective derivative thereof. Oxidation with either catalytic amounts of an TEMPO in the presence of stoichiometric amounts of a suitable co-oxidizing reagent i.e. sodium hypochlorite (NaOCl) leads mainly to the oxidation of primary alcohol groups to aldehydes (see FIG. 35, in case of HES either the position 6 or the terminal C atom of the hydroxyethyl group is converted to an aldehyde) without opening of the saccharide rings (lit: P. L. Brad, H. van Bekkum, A. C. Besemer, Topics in Catalysis, 2004, 27, 1-4; review: W. Adam, C. R. Saha-Moller, P. A. Ganeshpure, Chem. Rev. 2001, 101, 3499-3548 and cited papers, A. E. J. de Nooy, A. C. Besemer, H. v. Bekkum, Carbohydrate Research, 1995, 269, 89, EP 1 093 467, EP 1 173 409, WO 00/50621, EP 1 077 221, EP 1 149 346).

Alternatively, instead of catalytic amounts of TEMPO stoichiometric amounts of the active species—the oxoammonium compound—can be used (lit: J. M. Bobbitt, N. Merbouh, Organic Syntheses, 2005, 82, 80). Other TEMPO derivatives (i.e. 4-acetamido-, 4-hydroxy-TEMPO) are also suitable especially regarding the pH of the reaction or the solubility in water.

Following the oxidation the obtained aldehyde groups are converted to amines by reductive amination. As reducing agents e.g. sodium cyanoborohydride or a borane-dimethylamine complex (or other complex borane compounds) can be used. As amine compound e.g. ammonium chloride or diamines such as 1,3-diaminopropane, 1,3-diaminopropan-2-ol, or lysine can be used preferably at slightly acidic pH values. The usage of diamines enhances the spacer length between the HES backbone and the peptide drug and the yield of the reductive amination.

Not converted aldehyde groups with be reduced back again to the starting primary alcohol.

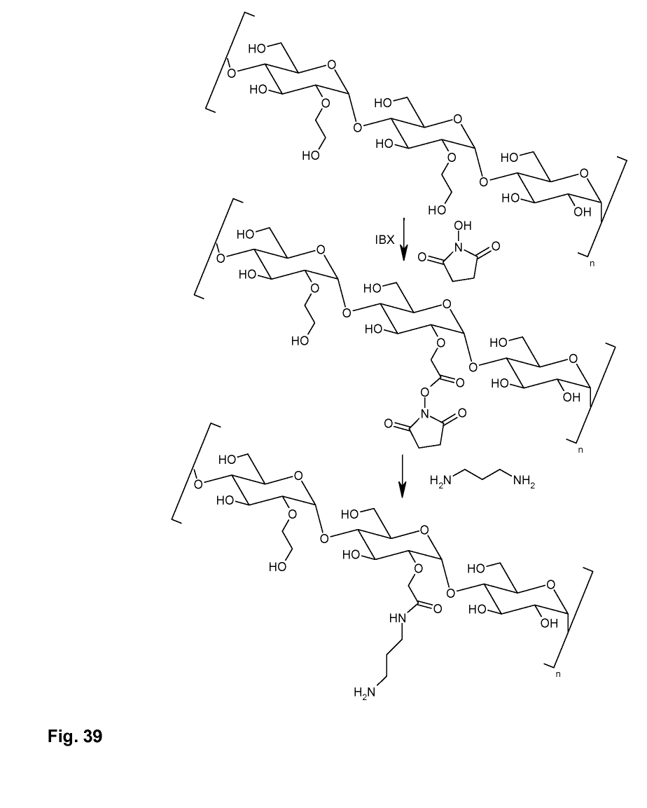

A modification, of the IBX oxidation can be done in DMSO in presence of N-hydroxysuccinimide (FIG. 39). In this case the corresponding activated ester of the uronic acid is directly formed. This species can be directly converted with i.e. diamines i.e. 1,3-diaminopropane to an aminated HES (ht R. Mazitschek, M. M Ibaier, A. Giannis, Angew. Chem. 2002, 114, 21, 4216-4218; A. Schulze, A. Glenn's, Adv. Synth. Catal. 2004, 346, 252-256).

2. A Two-Step Process where HES is Activated by a “Coupling” Reagent and Reacted with an Excess of a Bisfunctional Amine.

Several methods are described for the activation of polysaccharides i.e. 1,1′-carbonyldiimidazole (CDI) (lit: G. S. Bethell, J. S. Ayers, M. T. W. Hearn, W. S. Hancock, J. of Chromatography, 1981, 219, 361-372), epibromohydrine (1-bromo-2,3-epoxypropane, alternatively epichlorohydrine, respectively 1-chloro-2,3-epoxy-propane) (lit: H. Döbeli, E. Huchuli, Patent, 0253303 B1), 2,2,2-trifluoroethane-sulfonyl chloride (tresyl chloride) (lit: H. P. Jennissen, J. Mol. Recogn., 1995, 8, 116-124), bromocyanide (BrCN) G. S. Bethell, J. S. Ayers, M. T. VV. Hearn, W. S. Hancock, J. of Chromatography, 1981, 219, 361-372; H. P. Jennissen, J. Mol'. Recogn., 1995, 8, 116-124, H. Döbeli, E. Huchuli, Patent, EP0253303 B1). All reagents have in common that they introduce functional groups, which are highly reactive and can be reacted in a second step with an excess of bifunctional nucleophiles like amines i.e. ammonium chloride (not suitable with all activating reagents) or diamines i.e. 1,3-diaminopropane, 1,3-diaminopropan-2-ol, or lysine.

3. A One-Step Process where HES is Activated by Addition of a Suitable Amine Precursor.

Suitable amine precursors are e.g. halogenoalkylemines (i.e. as their salts, i.e. bromoethylammonium bromide) or reactive azarings i.e. aziridines i.e. lithium L-azindine-2-carboxylate.

All three described strategies have in common that after the introduction of the amino groups to the hydroxyethyl starch these amino groups are converted to maleimides as a suitable example of an appropriate linker. This can e.g. be accomplished by reacting with i.e. activated w-maleimido carboxylic acids i.e. 3-(maleimido)propionic acid N-hydroxysuccinimide ester or 4-(maleimido)butyric acid N-hydroxysuccinimide ester. The resulting maleimides represent the final active functional groups for coupling with peptides that bear a free thiol group.

### 1. A Two-Step Process: Aldehyde Groups are Introduced by Oxidation and Followed by Reductive Amination.

### 1.1. Oxidation of Primary Alcohols to Aldehydes

By direct oxidation of the primary alcohols in hydroxyethyl starch, more precisely the C6-OH groups of the glucose and the hydroxyethyl groups, aldehyde groups can be formed. These oxidation products are performed with commercially available oxidizing agents like TEMPO or IBX (e.g. Sigma-Aldrich or Acros).

a) Oxidation with Periodate

This method is described in more detail in the experimental section under C (see below)

b) Oxidation with TEMPO

By using 2,2,6,6-Tetramethylpiperidine-1-oxyl (TEMPO) or its derivatives, e.g. 4-acetamido-TEMPO or 4-hydroxy-TEMPO and co-oxidants like sodium hypochlorite in a mixture with potassium bromide (molar ratio TEMPO:NaOCl:KBr e.g. 1:40:20) the primary alcohols can be oxidized in short reaction times around 60 min in a phosphate buffer at a pH range between 6-8, whereby a higher pH increases the reaction speed. With different molar concentration of the oxidizing mixture, especially the co-oxidant, the number of formed aldehydes can be controlled. Consequently the amount of anchor groups and thus the amount of peptide drug on the carrier can be controlled by this first step.

The optimisation was monitored with the reagent Purpald that forms a purple adduct only with aldehydes and the redox titration of the remaining hypochlorite with an iodine/starch complex.

The working-up was performed by ultrafiltration techniques using a PES membrane of different molecular weight cut offs followed by lyophilisation (literature: P. L. Bragd, H. van Bekkum, A. C. Besemer, Topics in Catalysis, 2004, 27, 1-4: review: W. Adam, C. R. Saha-Moller, P. A. Ganeshpure, Chem. Rev. 2001, 101, 3499-3548 and cited papers; A. E. J. de Nooy, A. C. Besemer, H. v. Bekkum, Carbohydrate Research, 1995, 269, 89).

FIG. 35 gives an illustrating overview over the TEMPO mediated oxidation mechanism of primary alcohols. Further oxidation to carboxylates only occurs by usage of an excess of oxidizing reagent.

c) Oxidation with IBX

By using the oxidizing reagent 2-Iodoxybenzoic acid (IBX) or its derivatives, HES can be oxidized in DMSO as solvent. After 1-2 h reaction time the IBX can be removed by adding water (10 times) and the precipitated IBX is removed by filtration. The working-up was performed by ultrafiltration techniques using a PES membrane of different molecular weight cut offs followed by lyophilisation.

With different molar concentration of the IBX the number of formed aldehydes can be controlled. By that concentration the amount of anchor groups and so the amount of peptide drug on the carrier can be controlled as well.

The optimization was monitored with the reagent Purpald that forms a purple adduct only with aldehydes (for a review see: V. V. Zhdankin, Current Organic Synthesis, 2005, 2, 121-145 and cited papers)

FIG. 36 gives a schematic overview over the oxidation of primary alcohols with TEMPO or IBX followed by a reductive amination.

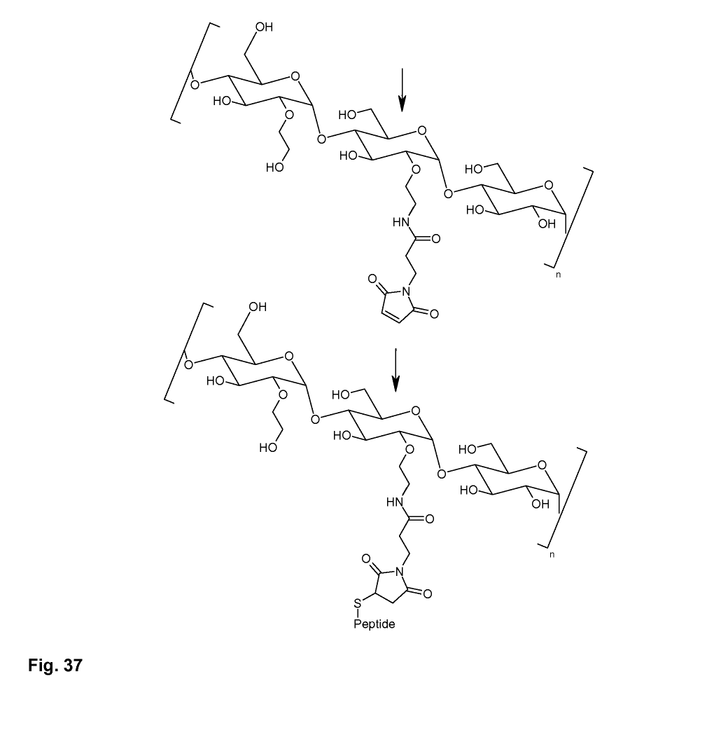

FIG. 37 illustrates the introduction of the maleimide groups and conjugation with a peptide drug.

### 1.2 Reductive Amination

a) Reductive Amination with Ammoniumchloride

This method is described in more detail in the experimental section of C (see below).

b) Reductive Amination with Diamine Linker

In order to enhance the spacer length between the HES backbone an the peptide drug and the yield of the reductive amination the reaction can be performed with a diamine, like 1,3-diaminopropane, 1,3-diaminopropan-2-ol or lysine as amine source and different reducing agents, i.e. sodiumcyanoborohydride or borane-dimethylamine complex.

### Example

### Reductive Amination with 1,3-diaminopropane

The reductive amination of the oxidised HES is performed in a 1M phosphate bugger pH=5 with an 10 times excess of 1,3-diaminopropane compared to the used oxidizing agent in the previous step. After equilibration for approximately 90 min an excess of sodiumcyanoborohydride (Na[CN]BH₃) is added in several portions. The working-up was performed by ultrafiltration techniques using a PES membrane of different molecular weight cut offs followed by lyophilisation. From the optimized HES derivatives only the molar mass range larger than 100 kDa were used.

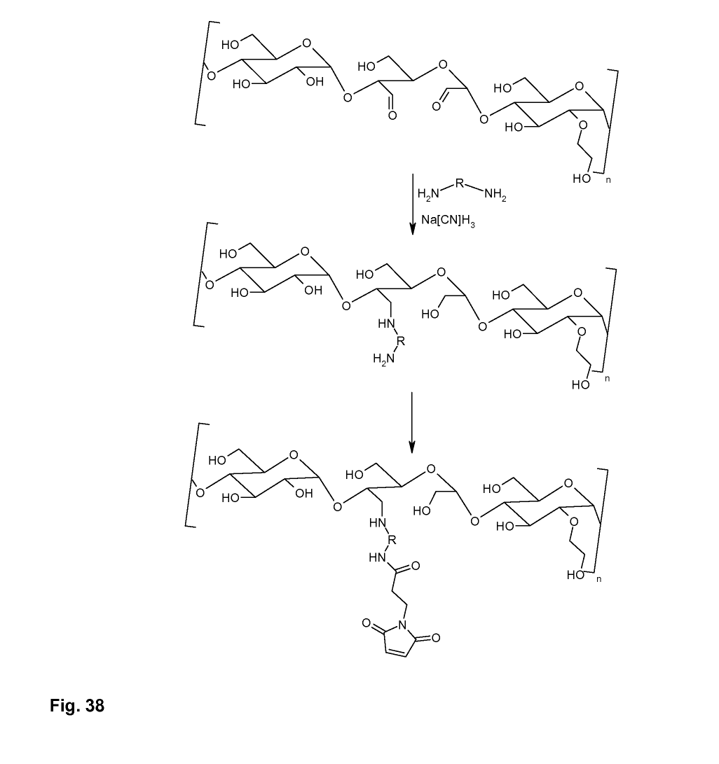

FIG. 38 gives an illustrating overview over the reductive amination with diamines like 1,3-diaminopropane followed by the introduction of the maleimide groups by the example of an periodate oxidised HES.

### Oxidation of Primary Alcohols Directly to Activated Esters

By direct oxidation of the primary alcohols in Hydroxyethyl starch, more precisely the C6-OH groups of the glucose and the hydroxyethyl groups. For these oxidation common commercially available oxidizing agents IBX (e.g. Sigma-Aldrich or Acros) in presence of N-hydroxysuccinimide (HOSu) the alcohol in oxidised to the OSu-ester, which can directly be converted into an amine by using a diamines.

Oxidation with IBX in Presence of HOSu—Direct Conversion to the Amine

By using the oxidizing reagent 2-Iodoxybenzoic acid (IBX) or its derivatives in the presence of N-hydroxysuccinimide. HES can be oxidized in DMSO as solvent. After 1-2 h the oxidation is over and to the formed OSu-ester an large excess (10 times) of the diamine is added, e.g. 1,3-diaminopropane.

The working-up was performed by ultrafiltration techniques using a PES membrane of different molecular weight cut offs followed by lyophilisation. From the optimized HES derivatives only the molar mass range larger than 100 kDa were used (for literature see: R. Mazitschek, M. Ibaier, A. Giannis, Angew. Chem. 2002, 114, 21, 4216-4218; A. Schulze, A. Giannis, Adv. Synth. Catai. 2004, 346, 252-256)

FIG. 39 illustrates the oxidation of primary alcohols to the OSu-ester followed by direct conversion with a diamine.

2. A Two-Step Process where HES is Activated by a “Coupling” Reagent and Reacted with an Excess of a Bisfunctional Amine.

Several alternatives may be applied to introduce amine groups onto the HES backbone. Some examples:

a) Modification with Carbonyldiimidazole (CDI)

Dried HES is suspended in dry acetone for 1 h. CDI is added and the mixture is stirred for 1 h. Alternatively some salts, e.g. potassium iodide, as activator/co-nucleophiles can be added. The HES is spun down (2000 U/min, >10 min). After decanting new acetone is added and the HES is spun down again. After 3 times washing with acetone the HES is taken up in 1M carbonate buffer pH 10 and 1,3-diaminopropane (pH 11) is added and the mixture was stirred for 1 h. The HES is worked up by ultracentrifugation (MWCO 100 kD) followed by lyophilisation.

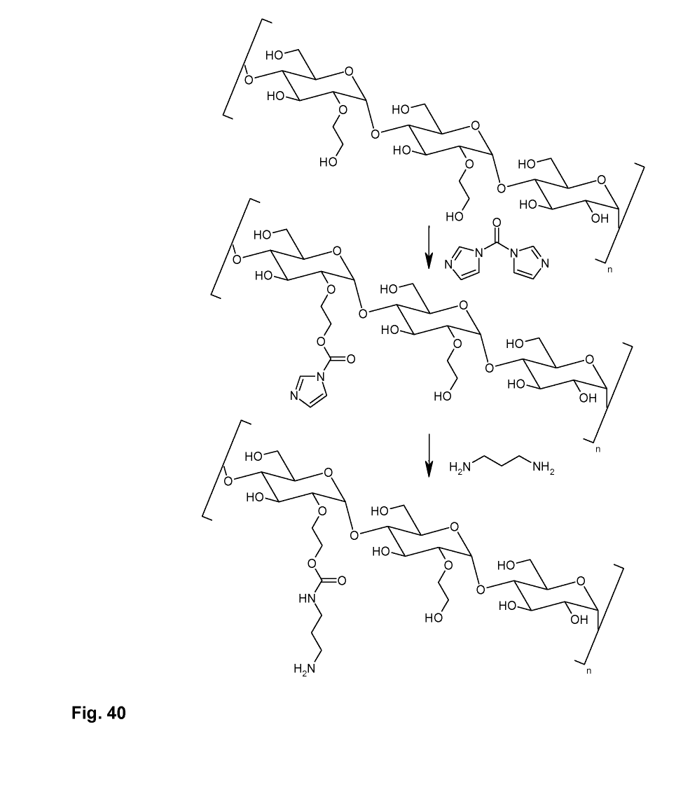

FIG. 40 illustrates the modification with carbonyldiimidazole followed by an diamine to introduce an amine group.

b) Modification with Epibromohydrine (Epi)

HES is dissolved in some DMF and epibromohydrin is added (alternatively some salts, e.g. potassium iodide, as activator/co-nucleophiles can also be added) and the mixture is stirred over night. The HES is diluted with water (10 times) and worked up by ultracentrifugation (MWCO 50 or 100 kD) followed by lyophilisation. The product is dissolved in 1M phosphate buffer pH=7 and 1,3-diaminopropane is added and the mixture is stirred for 1 h. The HES is worked up by ultracentrifugation (MWCO 50 or 100 kD) followed by lyophilisation.

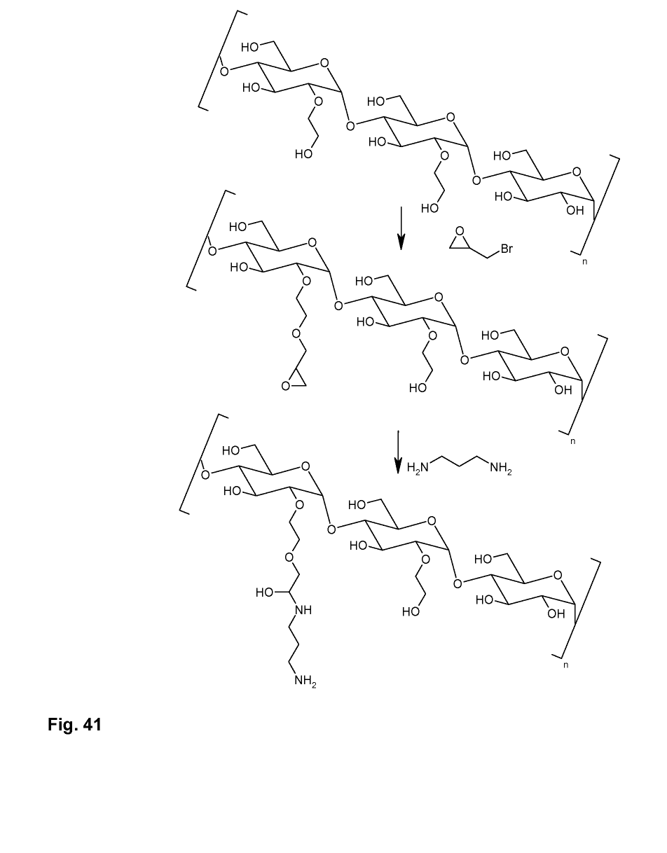

FIG. 41 illustrates the modification with epichlohydrine followed by a diamine to introduce an amine group.

3. A One-Step Process where HES is Activated by Addition of a Suitable Amine Precursor.
a) Modification with 2-aminoethyl Bromide Hydrobromide

HES is dissolved in DMSO and 2-bromoethylamin hydrobromid is added (alternatively some salts, e.g. potassium iodide, as activator/co-nucleophiles can also be added) and stirred over night (also some heating can be used). The HES is diluted with water (10 times) and worked up by ultracentrifugation (MWCO 50 or 100 kD) followed by lyophilisation.

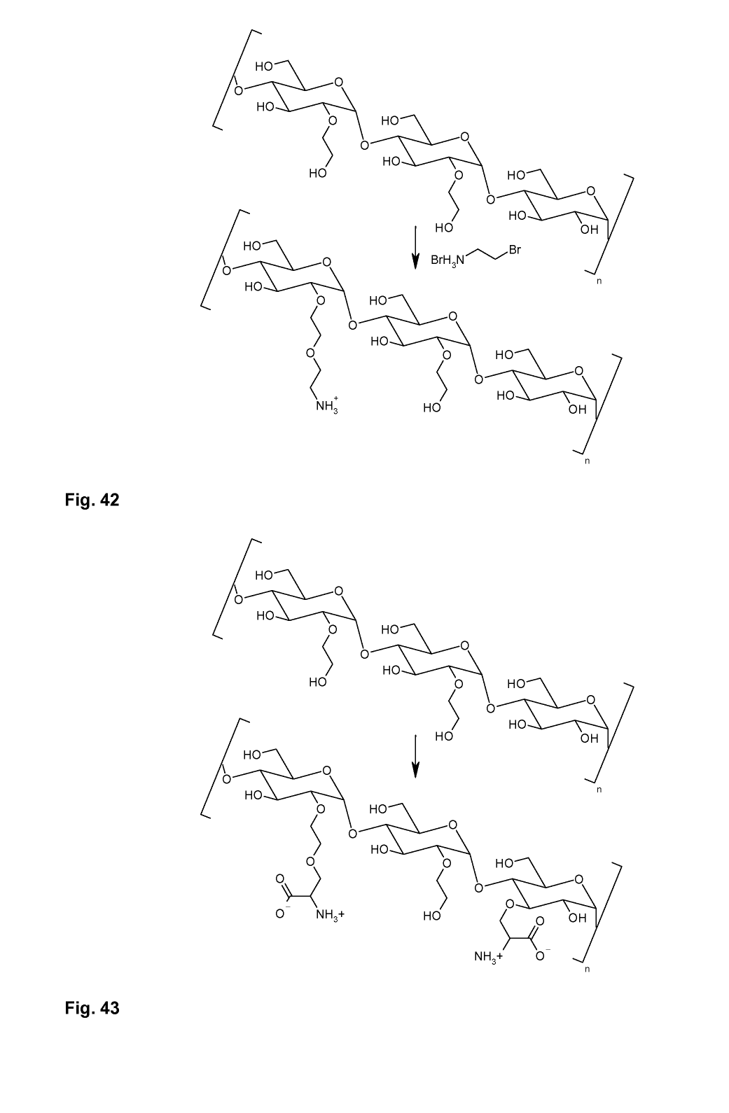

FIG. 42 illustrates the modification with 2-aminoethyl bromide hydrochloride to introduce an amine group.

b) Modification with Lithium L-Aziridine-2-Carboxylate

HES is dissolved in DMSO or a buffered aqueous solution and 2-lithium L-aziridine-2-carboxylate is added (alternatively some salts, e.g. potassium iodide, as activator/co-nucleophiles can also be added) and stirred over night. The mixture is diluted with water (10 times) and worked up by ultracentrifugation (MWCO 50 or 100 kD) followed by lyophilisation.

FIG. 43 illustrates the modification with lithium L-aziridine-2-carboxylate to introduce an amine group.

### C. Detailed Examples of Embodiments of the Present Invention

### I. Peptide Synthesis of Monomers

### Manual Synthesis

The synthesis is carried out by the use of a Discover microwave system (CEM) using PL-Rink-Amide-Resin (substitution rate 0.4 mmol/g) or preloaded Wang-Resins in a scale of 0.4 mmol. Removal of Fmoc-group is achieved by addition of 30 ml piperidine/DMF (1:3) and irradiation with 100 W for 3×30 sec. Coupling of amino acids is achieved by addition of 5 fold excess of amino acid in DMF PyBOP/HOBT/DIPEA as coupling additives and irradiation with 50 W for 5×30 sec. Between all irradiation cycles the solution is cooled manually with the help of an ice bath. After deprotection and coupling, the resin is washed 6 times with 30 ml DMF. After deprotection of the last amino acid some peptides are acetylated by incubation with 1.268 ml of capping solution (4.73 ml acetic anhydride and 8.73 ml DIEA in 100 ml DMSO) for 5 minutes. Before cleavage, the resin is then washed 6 times with 30 ml DMF and 6 times with 30 ml DCM. Cleavage of the crude peptides is achieved by treatment with 5 ml TFA/TIS/EDT/H₂O (94/1/2.512.5) for 120 minutes under inert atmosphere. This solution is filtered into 40 ml cold ether. The precipitate is dissolved in acetonitrile/water (1/1) and the peptide is purified by RP-HPLC (Kromasil 100 C18 10 μm, 250×4.6 mm).

### Automated Synthesis

The synthesis is carried out by the use of an Odyssey microwave system (CEM) using PL-Rink-Amide-Resins (substitution rate 0.4 mmol/g) or preloaded Wang-Resins in a scale of 0.25 mmol. Removal of Fmoc-groups is achieved by addition of 10 ml piperidine/DMF (1:3) and irradiation with 100W for 10×10 sec. Coupling of amino acids is achieved by addition of 5 fold excess of amino acid in DMF PyBOP/HOBT/DIPEA as coupling additives and irradiation with 50W for 5×30 sec. Between all irradiation cycles the solution is cooled by bubbling nitrogen through the reaction mixture. After deprotection and coupling, the resin is washed 6 times with 10 ml DMF. After deprotection of the last amino acid, some peptides are acetylated by incubation with 0.793 ml of capping-solution (4.73 ml acetic anhydride and 8.73 ml DIEA in 100 ml DMSO) for 5 minutes. Before cleavage the resin is then washed 6 times with 10 ml DMF and 6 times with 10 ml DCM. Cleavage of the crude peptides is achieved by treatment with 5 ml TFA/TIS/EDT/H₂O (94/1/2.5/2.5) for 120 minutes under an inert atmosphere. This solution is filtered into 40 ml cold ether, the precipitate dissolved in acetonitrile/water (1/1) and the peptide is purified by RP-HPLC (Kromasil 100 C18 10 μm, 250×4.6 mm).

### Purification

All peptides were purified using a Nebula-LCMS-system (Gilson). The crude material of all peptides was dissolved in acetonitrile/water (1/1) and the peptide purified by RP-HPLC (Kromasil 100 C18 10 μm, 250×4.6 mm). The flow rate was 20 ml/min and the LCMS split ratio 1/1000.

### II. Formation of Intramolecular Disulfide Bridges

Cyclization with K₃[(FeCN₆)

Solution 1: 10 mg of the peptide are dissolved in 0.1% TFA/acetonitrile and diluted with water until a concentration of 0.5 mg/ml is reached. Solid ammonium bicarbonate is added to reach a pH of app. 8.

Solution 2: in a second vial 10 ml 0.1% TFA/acetonitrile are diluted with 10 ml of water. Solid ammonium bicarbonate is added until a pH of 8 is reached and 1 drop of a 0.1 M solution of K₃[(FeCN₆)] is added.

Solution 1 and 2 are added dropwise over a period of 3 hours to a mixture of acetonitrile/water (1/1; pH=8). The mixture is incubated at room temperature overnight and the mixture concentrated and purified by LCMS.

Cyclization with CLEAR-OX™-Resin

To 100 ml of acetonitrile/water (1/1; 0.1% TFA), solid ammonium bicarbonate is added until a pH of 8 is reached. This solution is degassed by bubbling Argon for 30 minutes. Now 100 mg of CLEAR-OX™-resin is added. After 10 minutes, 10 mg of the peptide is added as a solid. After 2 h of incubation, the solution is filtered, concentrated and purified by LCMS.

### Purification of Cyclic Peptides:

All peptides were purified using a Nebula-LCMS-system (Gilson). The crude material of all peptides was dissolved in acetonitrile/water (1/1) or DMSO and the peptide was purified by RP-HPLC (Kromasil 100 C18 or C8 10 μm, 250×4.6 mm). The flow rate was 20 ml/min and the LCMS split ratio 1/1000.

III. In-Vitro Assays with Monomers
Proliferation assay with TF-1 Cells by BrdU Incorporation

TF-1 Cells in logarithmic growth phase (˜2.10⁵-1.10⁶ cells/ml; RPMI medium; 20% fetal calf serum; supplemented with Penicillin, streptomycin, L-Glutamine; 0.5 ng/ml Interleukin 3) are washed (centrifuge 5 min. 1500 rpm and resuspend in RPMI complete without IL3 at 500.000 cells/ml) and precultured before start of the assay for 24 h without IL-3. At the next day the cells are seeded in 24- or 96-well plates usually using at least 6 concentrations and 4 wells per concentration containing at least 10.000 cells/well per agent to be tested. Each experiment includes controls comprising recombinant EPO as a positive control agent and wells without addition of cytokine as negative control agent. Peptides and EPO-controls are prediluted in medium to the desired concentrations and added to the cells, starting a culture period of 3 days under standard culture conditions (37° C., 5% carbon dioxide in the gas phase, atmosphere saturated with water). Concentrations always refer to the final concentration of agent in the well during this 3-day culture period. At the end of this culture period, FdU is added to a final concentration of 8 ng/ml culture medium and the culture continued for 6 hours. Then, BrdU (bromodeoxyuridine) and dCd (2-deoxycytidine) are added to their final concentrations (10 ng/ml BrdU; 8 ng/ml dCD; final concentrations in culture medium) and culture continued for additional 2 hours.

At the end of this incubation and culture period, the cells are washed once in phosphate buffered saline containing 1.5% BSA and resuspended in a minimal amount liquid. From this suspension, cells are added dropwise into 70% ethanol at −20° C. From here, cells are either incubated for 10 min on ice and then analyzed directly or can be stored at 4° C. prior to analysis.

Prior to analysis, cells are pelleted by centrifugation, the supernatant is discarded and the cells resuspended in a minimal amount of remaining fluid. The cells are then suspended and incubated for 10 min. in 0.5 ml 2M HCl/0.5% triton X-100. Then, they are pelleted again and resuspended in a minimal amount of remaining fluid, which is diluted with 0.5 ml of 0.1 N Na₂B₄0₇, pH 8.5 prior to immediate repelleting of the cells. Finally, the cells are resuspended in 40 μl of phosphate buffered saline (1.5% BSA) and divided into two reaction tubes containing 20 μl cell suspension each. 2 μl of anti-BrdU-FITC (DAKO, clone Bu20a) are added to one tube and 2 μl control mlgG1-FITC (Sigma) are added to the second tube starting an incubation period of 30 min. at room temperature, Then, 0.4 ml of phosphate buffered saline and 10 μg/ml Propidium Iodide (final concentration) are added. Analysis in the flow cytometer refers to the fraction of 4C cells or cells with higher ploidy and to the fraction of BrdU-positive cells, thus determining the fraction of cells in the relevant stages of the cell cycle.

Proliferation Assay with TF-1 Cells by MU

TF-1 Cells in logarithmic growth phase (˜2.10⁵-1.10⁶ cells/ml; RPMI medium; 20% fetal calf serum; supplemented with Penicillin, streptomycin, L-Glutamine; 0.5 ng/ml Interleukin 3) are washed (centrifuge 5 min. 1500 rpm and resuspended in RPMI complete without IL3 at 500.000 cells/ml) and precultured before start of the assay for 24 h without IL-3. At the next day the cells are seeded in 24- or 96-well plates usually using at least 6 concentrations and 4 wells per concentration containing at least 10.000 cells/well per agent to be tested. Each experiment includes controls comprising recombinant EPO as a positive control agent and wells without addition of cytokine as negative control agent. Peptides and EPO-controls are prediluted in medium to the desired concentrations and added to the cells, starting a culture period of 3 days under standard culture conditions (37° C., 5% carbon dioxide in the gas phase, atmosphere saturated with water). Concentrations always refer to the final concentration of agent in the well during this 4-day culture period.

At day 4, prior to start of the analysis, a dilution series of a known number of TF-1 cells is prepared in a number of wells (0/2500/5000/10000/20000/50000 cells/well in 100 μl medium). These wells are treated in the same way as the test wells and later provide a calibration curve from which cell numbers can be determined. Having set up these reference wells, MTS and PMS from the MTT proliferation kit (Promega, CellTiter 96 Aqueous non-radioactive cell proliferation assay) are thawed in a 37° C. water bath and 100 μl of PMS solution are added to 2 ml of MTS solution. 20 μl of this mixture are added to each well of the assay plates and incubated at 37° C. for 3-4 h. 25 μl of 10% sodium dodecyl sulfate in water are added to each well prior to measurement E492 in an ELISA Reader.

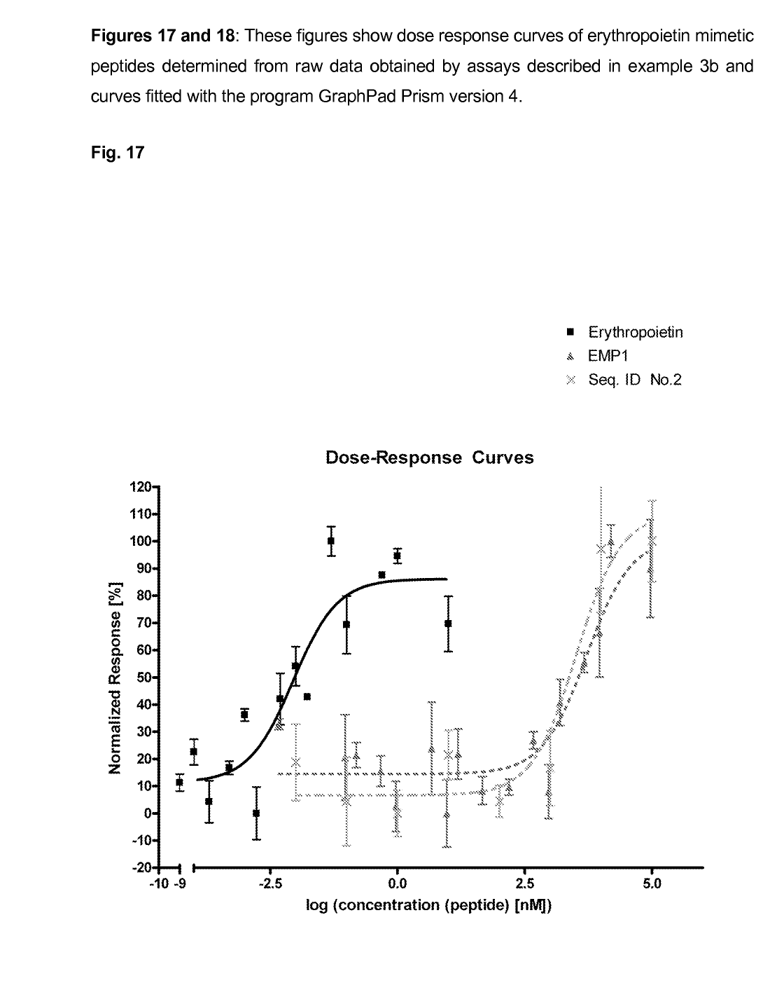

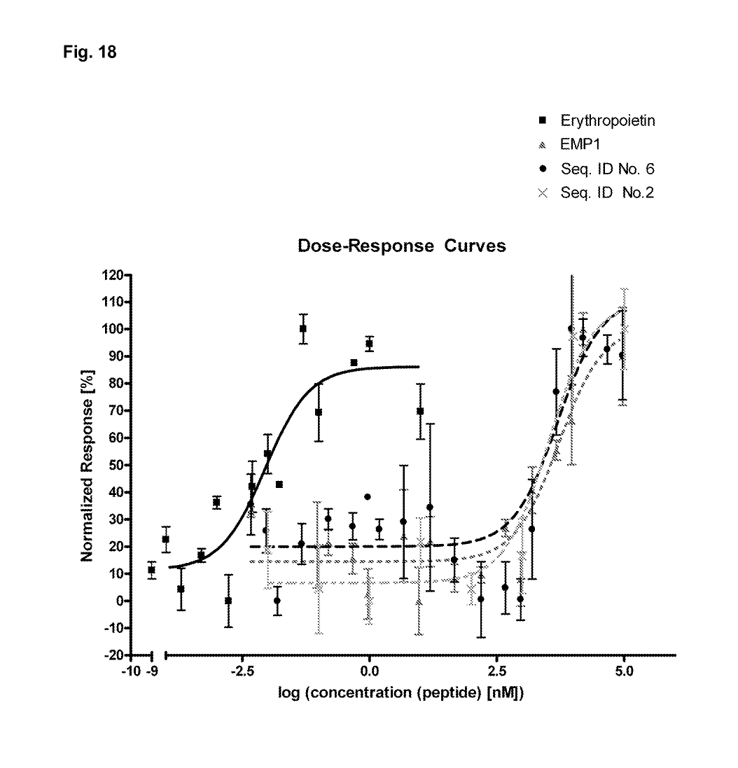

Using graphical evaluations as shown in FIGS. 17 and 18 based on calculations of the dose-response relationship using the program GraphPad the following EC50 values were determined on the basis of MU-assay data:

The following table shows the EC₅₀ values of some exemplary peptide monomers:

| | | |
|---|---|---|
| SEQ ID NO 2: | GGTYSCHFGKLTWVCKKQGG | 3284 nmol/l |
| SEQ ID NO 4: | GGTYSCHFGKLTWVCKPQGG | 4657 nmol/l |
| SEQ ID NO 5: | GGTYSCHFGRLTWVCKPQGG | 5158 nmol/l |
| SEQ ID NO 6: | GGTYSCHFGRLTWVCKKQGG | 4969 nmol/l |
| SEQ ID NO 7: | GGTYSCHF-(Als)-LTWVCKPQGG | 5264 nmol/l |
| SEQ ID NO 8: | GGTYSCHF-(Als)-LTWVCKKQGG | 4996 nmol/l |
| | GGTYSCHFGPLTWVCKKQGG | 2518 nmol/l |
| | GGTYSCHFAKLTWVCKKQGG | 5045 nmol/l |
| | GGTYSCHFGGLTWVCKPQGG | no activity |
| | | detectable |

### IV. Synthesis of Bivalent EPO Mimetic Peptide Units

### Automated Synthesis of Linear SEQ ID NO 11 (AGEM11)

The synthesis is carried out by the use of a Liberty microwave system (CEM) using Rink-Amide-Resin (substitution rate 0.19 mmol/g) in a scale of 0.25 mmol. Removal of Fmoc-groups is achieved by double treatment with 10 ml piperidine/DMF (1:3) and irradiation with 50W for 10×10 sec. Coupling of amino acids is achieved by double treatment with a of 4 fold excess of amino acid in DMF PyBOP/HOBT/DIPEA as coupling additives and irradiation with 50 W for 5×30 sec. Between all irradiation cycles the solution is cooled by bubbling nitrogen through the reaction mixture. After deprotection and coupling, the resin is washed 6 times with 10 ml DMF. After the double coupling cycle all unreacted amino groups are blocked by treatment with a 10 fold excess of N-(2-Chlorobenzyloxycarbonyloxy)succinimide (0.2M solution in DMF) and irridation with 50W for 3×30 sec. After deprotection of the last amino acid, the peptide is acetylated by incubation with 0.793 ml of capping-solution (4.73 ml acetic anhydride and 8.73 ml DIEA in 100 ml DMSO) for 5 minutes. Before cleavage the resin is then washed 6 times with 10 ml DMF and 6 times with 10 ml DCM. Cleavage of the crude peptides is achieved by treatment with 5 ml TFA/TIS/EDT/H₂O (94/1/2.5/2.5) for 120 minutes under an inert atmosphere. This solution is filtered into 40 ml cold ether, the precipitate dissolved in acetonitrile/water (1/1) and the peptide is purified by RP-HPLC (Kromasil 100 C18 10 μm, 250×4.6 mm).

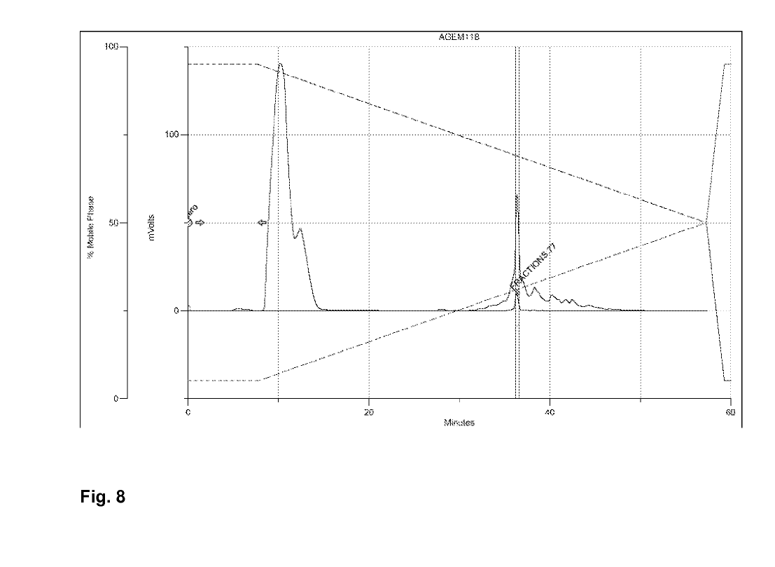

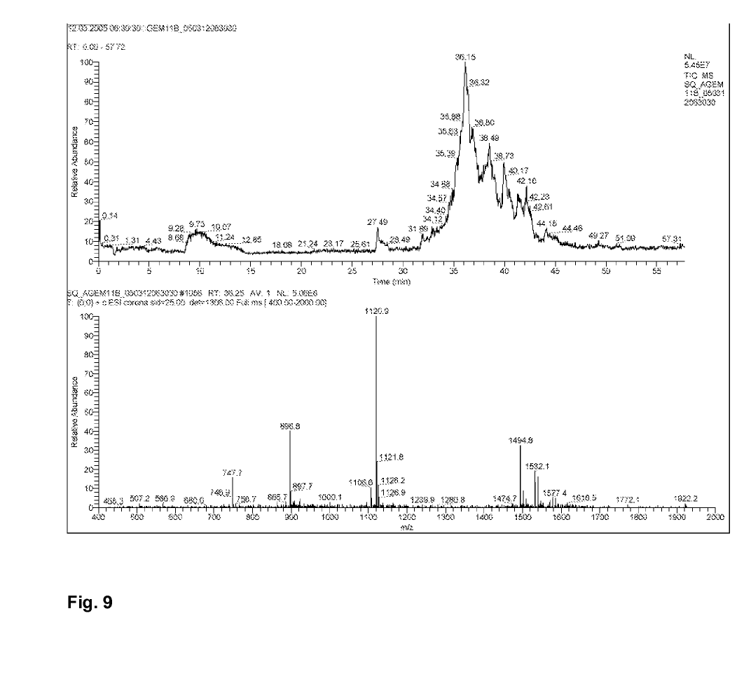

The purification scheme of linear AGEM11, Kromasil 100 C18 10 μm, 250×4.6 mm and the gradient used therefore is depicted in FIGS. 8 and 9 from 5% to 50% acetonitrile (0.1% TFA) in 50 minutes

### Cyclization of Linear AGEM11

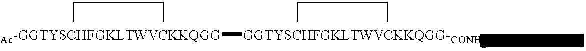

30 mg of the linear peptide are dissolved in 60 ml solution A. This solution and 60 ml DMSO are added dropwise to 60 ml solution A (total time for addition: 3 h). After 48 h the solvents are removed by evaporation and the remaining residue solved in 30 ml DMSO/water (1/1). 30 ml acetic acid and 17 mg iodine (solved in DMSO I water (1/1) are added and the solution is mixed for 90 minutes at room temperature. Afterwards 20 mg ascorbic acid are added and the solvents removed by evaporation. The crude mixture is solved in acetonitrile/water (2/1) and the peptide is purified by RP-HPLC (Kromasil 100 C18 10 μm, 250×4.6 mm).

Solution A: Acetonitrile/water (1/1) containing 0.1% TFA. The pH is adjusted to 8.0 by the addition of ammonium bicarbonate.

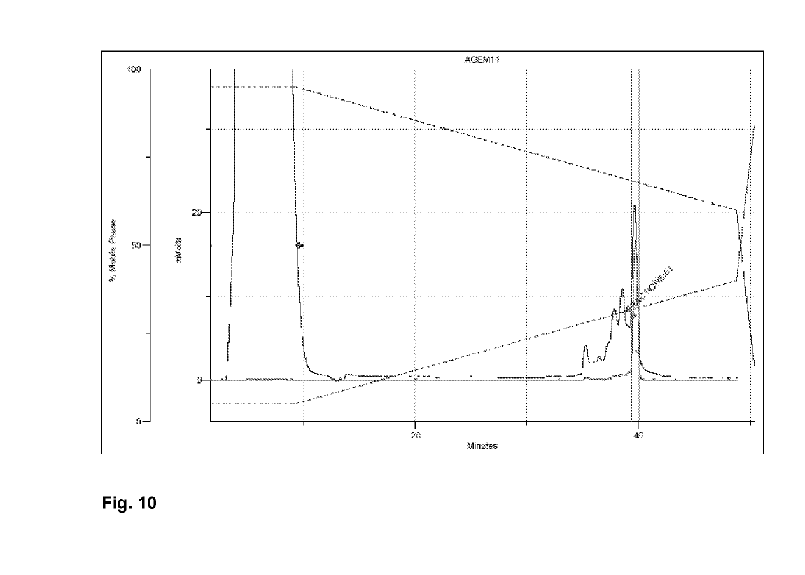

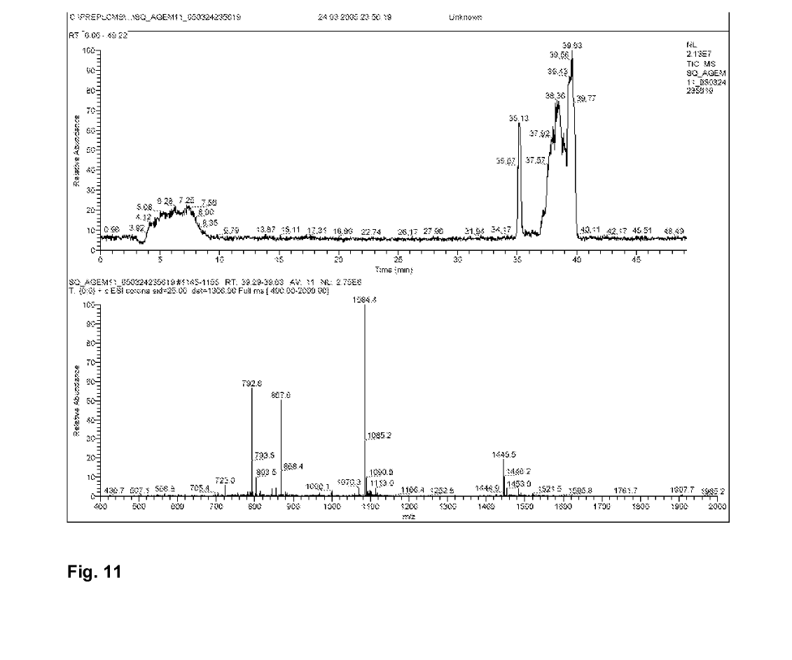

The purification parameters for cyclic AGEM11 are given in FIGS. 10 and 11 (scheme: Purification of cyclic AGEM11, Kromasil 100 C18 10 μm, 250×4.6 mm, gradient from 5% to 35% acetonitrile (0.1% TFA) in 50 minutes).

### V. In Vitro Proliferation Assay to Determine EPO Activity

TF1 cells in logarithmic growth phase (2.10⁵-1.10⁶ cells/ml grown in RPM1 with 20% fetal calf serum (FCS) and 0.5 ng/ml IL-3) were counted, and the number of cells needed to perform an assay were centrifuged (5 min. 1500 rpm) and resuspended in RPMI with 5% FCS without IL-3 at 300 000 cells/ml. Cells were precultured in this (starvation) medium without IL-3 for 48 hours. Before starting the assay the cells were counted again.

Shortly before starting the assay stock solutions of peptides and EPO were prepared. Peptides were weighed and dissolved in RPM with 5% FCS up to a concentration of 1 mM, 467 μM or 200 μM. EPO stock solutions were 10 nM or 20 nM. Two hundred ninety two μl of these stock solutions were pipetted into one well of a 96 well culture plate—one plate was taken for each substance to be tested. Two hundred μl of RPMI with 5% FCS were pipetted into seventeen other wells in each plate. Ninety two μl of stock solution were pipetted into a well containing 200 μl medium. The contents were mixed, and 92 μl from this well was transferred to the next, and so forth. This way a dilution series (18 dilutions) of each substance was prepared such that in each consecutive well the concentration was 1:√10 of the concentration in the well before that. From each well 3×50 μl was transferred to three empty wells. This way each concentration of substance was measured in quadruplicate. Note that the uppermost and lowermost line of wells of each plate was left void.

Pretreated (starved) cells were centrifuged (5 min. 1500 rpm) and resuspended in RPMI with 5% FCS at a concentration of 200 000 cells per ml. Fifty μl of cell suspension (containing 10 000 cells) was added to each well. Note that due to the addition of the cells the final concentrations of the substances in the wells were half that of the original dilution range. Plates were incubated for 72 h at 37° C. in 5% CO₂. Before starting the evaluation, a dilution range of known amounts of TF-1 cells into wells was prepared: 0/2500/5000/10000/20000/50000 cells/well were pipetted (in 100 μl RPMI+5% FCS) in quadruplicate.

To measure the number of live cells per well, ready-to-use MTT reagent (Promega, CellTiter 96 Aqueous One Solution Cell Proliferation Assay) was thawed in a 37° C. water bath. Per well, 20 μl of MTT reagent was added, and plates were incubated at 37° C. in 5% CO₂ for another 1-2 h. Twenty five μl of a 10% SDS solution was added, and plates were measured in an ELISA reader (Genios, Tecan). Data were processed in spreadsheets (Excel) and plotted in Graphpad.

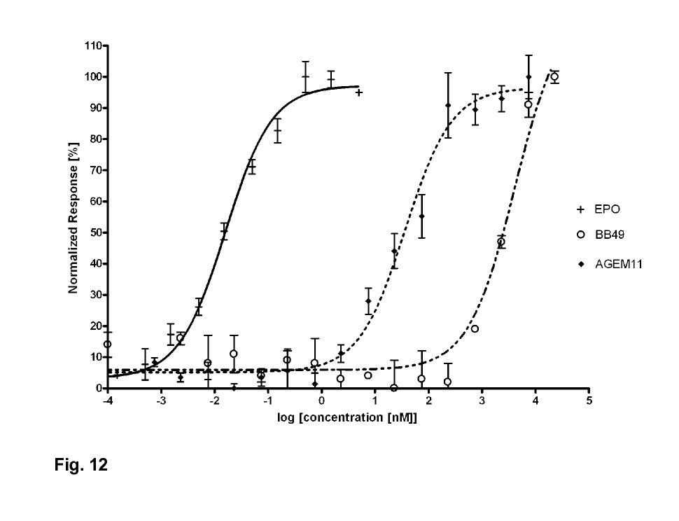

The data are summarized in FIG. 12.

| | ED50 (nM): |
|---|---|
| EPO | 0.0158 |
| BB49 (monomer, SEQ. ID NO 2) | 4113 |
| AGEM11 (bivalent) | 36.73 |

### VI. Extended Peptide Assays

In an extended assay, approximately 200 peptide sequences were tested for their EPO mimetic activity.

The peptides were synthesized as peptides amides on a LIPS-Vario synthesizer system. The synthesis was performed in special MTP-synthesis Plates, the scale was 2 μmol per peptide. The synthesis followed the standard Fmoc-protocol using HOBT as activator reagent. The coupling steps were performed as 4 times coupling. Each coupling step took 25 min and the excess of amino acid per step was 2.8. The cleavage and deprotection of the peptides was done with a cleavage solution containing 90% TFA, 5% TIPS, 2.5% H₂O and 2.5% DDT. The synthesis plate containing the finished peptide attached to the resin was stored on top of a 96 deep well plate. 50 μl of the cleavage solution was added to each well and the cleavage was performed for 10 min, this procedure was repeated three times. The cleaved peptide was eluted with 200 μl cleavage solution by gravity flow into the deep well plate. The deprotection of the side chain function was performed for another 2.5 h within the deep well plate. Afterwards the peptide was precipitated with ice cold ether/hexane and centrifuged. The peptides were solved in neutral aqueous solution and the cyclization was incubated over night at 4° C. The peptides were lyophilized.

FIG. 19 gives an overview over the synthesised and tested peptides monomers.

The peptides were tested for their EPO mimetic activity in an in vitro proliferation assay. The assay was performed as described under V. On each assay day, 40 microtiter plates were prepared for measuring in vitro activity of 38 test peptides, 1 reference example, and EPO in parallel. EPO stocks solutions were 20 nM.

The results are given in FIG. 19. As can be seen from the results, the tested peptides not fulfilling the consensus of the present invention did not depict EPO mimetic activity.

### VII. Synthesis of Peptide HES-Conjugates Using Periodate Oxidation

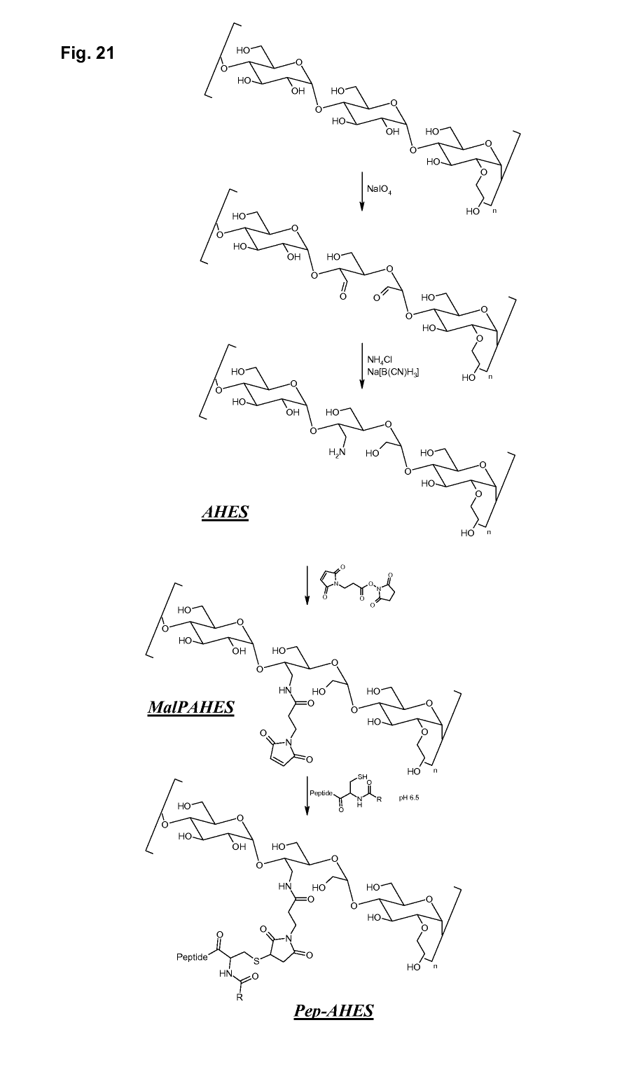

The principle reaction scheme is depicted in FIG. 21.

The aim of the described method is the production of a derivative of a starch, according to this example HES, which selectively reacts with thiol groups under mild, aqueous reaction conditions. This selectivity is reached with maleimide groups.

HES is functionalised first with amino groups and converted afterwards to the respective maleimide derivative. The reaction batches were freed from low molecular reactants via ultrafiltration membranes. The product, the intermediate products as well as the educts are all poly-disperse.

### Synthesis of Modified HES

Hydroxyethylstarch (Voluven® 130/0.4 or Serumwerk Bernburg 200/0.5) was attained via diafiltration and subsequent freeze-drying. The average molar weight was approximately 130 kDa with a molar degree of substitution of 0.4, respectively 200 kD, MS=0.5.

The synthesis was performed according to the synthesis described for amino dextran in the dissertation of Jacob Piehler, “Modifizierung von Oberflächen für die thermodynamische and kinetische Charakterisierung biomolekularer Erkennung mit optischen Transducern”, 1997, herein incorporated by reference. HES was activated by partial, selective oxidation of the click hydroxyl groups to aldehyde groups with sodium periodate as described in Floor et. al (1989). The aldehyde groups were converted via reductive amination with sodiumcyanoborhydride (NaCNBH₃) in the presence of ammonia to amino groups (Yalpani and Brooks, 1995).

### 1.1 Oxidation of Primary Alcohols to Aldehydes

### a) Periodate Oxidation/Opening

By a mild oxidation of the 1,2-dials in the saccharide by sodium periodate in water is aldehyde groups are introduced. By using different molar concentration of the oxidizing agent the number of available anchor groups and so the amount of peptide drug on the carrier can be controlled. To optimize the protocol the oxidation was monitored with the reagent Purpald that forms a purple adduct only with aldehydes. The reaction time can be reduced down to 8-18 h. The used amount of periodate represents 20% of the number of glucose building blocks (applying a glucose building block mass of 180 g/mol, DS=0.4). The working-up was performed via ultra filtration and freeze-drying. The purification of each polymeric product was performed by ultrafiltration techniques using a PES membrane of different molecular weight cut offs followed by lyophilisation. From the optimized HES derivatives only the molar mass range larger than 100 kDa were used

### Aldehyde Analysis

### Qualitative/Semi-quantitative: Purpald Reaction of the Available Aldehyde Groups

### 1.2 Reductive Amination

a) Reductive Amination with Ammonium Chloride

In the following step the introduced aldehyde groups were converted into amines by a reductive amination in a saturated solution of ammonium chloride at a slightly acidic pH value with sodium cyanoborohydride.

To optimize the protocol the aldehyde groups of the starting material were followed by the Purpald reagent and the formed amines with TNBS. These experiments have shown that the formation of the imine intermediate is in an equilibrium after a starting period and the added reducing agent prefers the imins over the aldehyde. So could be found that the optimal reaction is performed by several addition of the reducing agent with a total reaction time of 24 h.

Working-up via precipitation of the product or ultrafiltration.

### Amine Analysis

Qualitative: Ninhydrin Reaction (Kaiser-Test)

Quantitative: with 2,4,6-trinitrobenzole sulphonic acid (TNBS) in comparison with an amino dextrane.

The achieved substitution grade was around 2.8%. This results in a molar mass of one building block carrying one amino group of approx. 6400 g/mol.

Synthesis of maleimidopropionyl-amino-hydroxyethylstarch (“MaiPA-HES”)

After the amino groups are introduced (several different strategies exist, for examples see above), the anchor group maleimide is introduced with w-maleimido alkyl (or aryl) acid-N-hydroxysuccinimidesters.

### Synthesis

The final introduction of the maleimide groups into the HES is performed with 3-maleimidopropione acid-N-hydroxysuccinimidester (MalPA-OSu). When using an excess (5 to 10-fold) in a slightly acidic buffer the conversion is quantitatively (50 mM phosphate buffer, pH 6 or 7, 20% DMF, over night). The ultrafiltrated and lyophilized product is stored at −18° C.

### Analysis

The reaction of the amino group was verified with ninhydrin and TNBS. The number of introduced maleimide groups is demonstrated by reaction of gluthation (GSH) and the detection of excessive thiol groups with Ellmans reagent (DNTB) and via 700 MHz-¹H-NMR-spectroscopy.

The achieved substitution grade was around 2% and corresponds to 8500 g/mol per maleimide building block (180 g/mol glucose building block mass, MS=0.4).

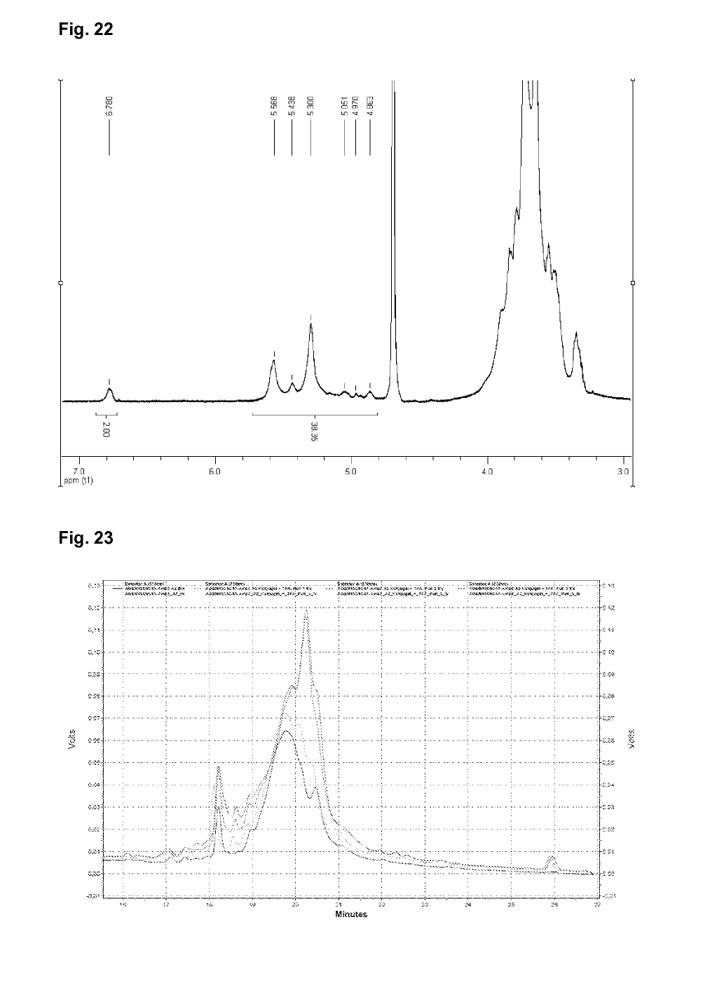

FIG. 22 shows a ¹H-NMR spectra (D₂O, 700 MHz) of a maleimide modified HES. Ratio of the maleimide proton (6.8 ppm) to the anomeric C—H (4.8-5.6 ppm) gives a building block size of approx. 6,900 g/mol (in comparison: the GSH/DNTB test gave 7,300 g/mol).

The number of maleimide groups and so the building block size can be measured by saturation with GSH and reaction with DNTB. The formed yellow colour is significant and can be quantified easily. These values give reliable building block sizes in between 5,000 and 100,000 g/mol depending on the used starting material, respectively the amount of periodate in the oxidation step. This method has been validated by ¹H-NMR spectroscopy of the product. In the NMR the content of maleimide groups can be quantified from the ratio of all anomeric C—H signals and the maleimide ring protons.

TABLE 1
Examples for the reachable virtual building block size of the
anchor group in the HES backbone via the periodate oxidation.

| Amount of periodate | |
|---|---|
| (1^{st} step) (eq) | Building block sizes maleimide (g/mol) |
| 0.01-0.03 | >55,000 |
| 0.02-0.04 | ~35,000-50,000 |
| 0.04-0.1 | ~15,000-35,000 |
| 0.1-0.3 | ~6,000-7,000 |

### Peptide-Hydroxyethyl Starch-Conjugate (Pep-AHES)

### Synthesis

A cysteine containing peptide was used which had either a free (Pep-IA) or a biotinytated (Pep-IB) N-term. A 4:1 mixture of Pep-IA/B was converted over night in excess (approx. 6 equivalents with MalPA-HES in phosphate-buffer, 50 mM, pH 6.5/DMF 80:20; working up occurred with ultra filtration and freeze-drying.

### Analysis

The UV-absorption was determined at 280 nm and the remaining content of maleimide groups was determined with GSH/DNTB.

The peptide yield was almost quantitative. Nearly no free maleimide groups were detectable.

For the conjugation of the peptide drug a peptide domain

Ac-GGTYSCHFGKLT-Na1-VCKKQRG-Am (BB68)

is used for creating a peptide unit by introducing a free thiol group (e.g. by introducing a cysteine residue at the N-terminus) as in

Ac-C(tBu)-GGTYSCHFGKLT-Na1-VCKKQRG-GGTYSCHFGKLT-
Na1-VCKKQRG-Am
(AGEM40)

an 10-50% excess of the deprotected peptide is conjugated in a slightly acidic buffer for 1-2 h. The conditions have been optimized to assure on the one hand that the HES backbone, the maleimide groups and the disulfide bridges are stable and on the other hand to observe a quantitative conversion. By using different maleimide functionalized HES compounds AplaGen could synthesize a number of supravalent EPO-Mimetic Peptides, which have shown in vitro a supravalent effect. Some examples are given below

TABLE 2
Supravalent EPO-mimetic Peptide conjugates
of AGEM40 with different peptide contents.

| | | Peptide | Peptide |
|---|---|---|---|
| Supravalent EPO- | Building block | content | content |
| Mimetic Peptide | sizes maleimid | theoretical | experimental |
| on HES | groups (g/mol) | (%) | (%) |
| AGEM40-HES A2 | 7,300 | 39 | 37 |
| AGEM40-HES A3 | 16,000 | 23 | 22 |
| AGEM40-HES A4 | 44,000 | 10 | 10 |

An easy chemical analysis of the supravalent EPO-mimetic peptide conjugates was realized in two steps. First the content of peptide was quantified by HPLC after a soft hydrolysis of the HES backbone and second the amount of polysaccharide was measured by a colorimetric test with phenol after a complete hydrolysis by sulphuric acid.

FIG. 23 shows a HPLC chromatogram (Shimadzu HPLC) of the TFA/water hydrolysis of the Supravalent EPO-Mimetic Peptide conjugates AGEM40-ARES A2. After a certain time the UV absorbance of all peptide containing species is constant at a maximal value and by comparison with the free peptide a peptide content of 37% can be calculated (theoretical value: 39%).

### VIII. Further In Vitro Experiments

Many of the experiments described below were already described above. However, the following details give a summarising overview over the described tests and results. Predominantly the human cell culture and bone marrow assays are discussed.

On one hand, rapid cell-line based assays were used to check for potency of optimised peptide sequences throughout the early stages of optimisation. These cell culture assays are still valid as rapid tests of efficacy of a new peptide or a new batch. The two endpoints, which were used for the cell line TF-1 (human cells) are proliferation (here usually determined as number of living cells at defined time points) and differentiation as marked haemoglobin production in TF-1 cells.

In addition, primary cells (human bone marrow stem cells) were used for CFU-assays, which are very close to the in vivo situation. They give answers to erythropoietic activity in case of the use of EPO mimetic peptides as peptide units in a much more in vivo-like fashion. However, they are to be handled more sophisticated and need more time per assay than the cell culture assays.

### Assays Using Human TF-1 Cells

TF-1 is a human erythroleukemia cell line that proliferates only in response to certain cytokines such as IL3 or EPO. In addition, TF-1 cells can differentiate towards an erythroid phenotype in response to EPO. TF-1 cells were obtained from DSMZ (Braunschweig, Germany). A product sheet is available at the DSMZ web site dsmz.de. TF-1 is the cell line recommended for EPO-activity assessment by the European Pharmacopoe.

Our internal culture protocol for maintenance culture:

| | |
|---|---|
| Medium: | RPMI + P/S + AmphoB + L-Glut. + 20% FCS + h-IL-3 |
| 1. | 500 ml RPMI + 5 ml P/S + 5 ml AmphoB |
| 2. | 200 ml RPMI + PS/AmphoB + 2.5 ml L-Glutamin + |
| | 50 ml FCS = complete Medium (1 month 4° C.) |
| 3. | 45 ml complete Medium + 22.5 ul h-IL-3 (1 week 4° C.) |
| Culture: | Maintain between 200.000 and 1.000.000 cells/ml |
| | For 3 days 2 × 10⁵/ml |
| | For 2 days 3 × 10⁵/ml |
| | For 1 days 5 × 10⁵/ml |

### Design of a TF-1 Proliferation Assay

In a TF-1 proliferation assay, TF-1 cells are seeded and cultured for several days in varying concentrations of EPO or EPO mimetic peptides in a multi-well plate.

For optimal results TF-1 cells should be cultured for two days in the absence of any cytokine (starved) before starting the assay. Three days after starting the assay, cell proliferation is measured indirectly by assaying the number of viable cells.

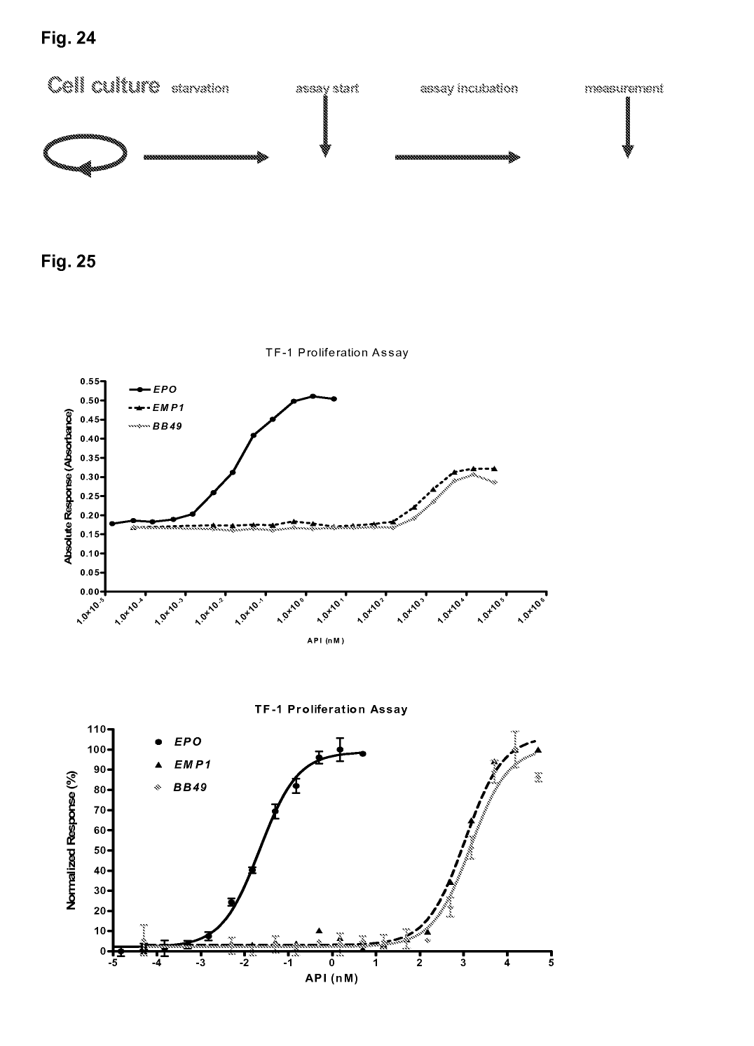

A tetrazolium reagent, called MTS, is added which is reduced to coloured formazan. This reaction depends on NADH and NADPH, in other words depends on mitochondrial activity. The amount of formazan is measured spectrophotometrically. Using a range of known cell numbers for calibration, it is possible to determine the absolute number of viable cells present under each condition. The principal design is also illustrated in FIG. 24.

The activity of a certain agent in this assay is determined by:
1. assessing whether this agent causes an increase in the number of viable cells at a certain concentration, and
2. at which concentration this agent exerts a half-maximum effect (determination of the EC50).

### Results of TF-1 Proliferation Assays

As a general remark, it has to be mentioned that all EPO-mimetic peptides (EMP1 and the proline modified peptides described above) behave in this assay as partial agonists, i.e. the maximal response is weaker than the response seen with EPO. Nevertheless, the assay can be used to determine the right/left shift in normalized plots and thus to determine the outcome of optimisations.

The first graph depicts this effect in absolute response without normalisation. All other graphs show normalized plots, which allow determination of EC50 values from the curves.

Two reference substances were used in the assays:
1) EMP1, a published peptide sequence with known EPO-mimetic properties (Johnson et al, 1997).
2) Recombinant Human Erythropoietin (EPO), was bought in the pharmacy as the Ortho Biotech product Epoetin alfa (Tradename in Germany: Erypo^{R})

The plots of these substances are given as black lines, continuous for EPO and dotted for EMP1.

The proline-modified EPO mimetic peptides are shown in the next Figs. as coloured continuous lines. These modified peptides depict the following sequence:
1) BB49

Ac-GGTYSCHFGKLTWVCKKQGG

shows an efficacy and potency in the same range as EMP1
2) BB68

Ac-GGTYSCHFGKLT-Na1-VCKKQRG-Am

is even more effective than EMP1 and 8849
3) AGEM40,

Ac-C(tBu)-GGTYSCHFGKLT-Na1-VCKKQRG-GGTYSCHFGKLT-
Na1-VCKKQRG-Am

which is a bivalent continuous peptide, which was designed based on the sequence of BB68 depicting improved features.
4) AGEM40_HES, which is an advanced, highly effective and potent peptide (AGEM40) HESylated according to the supravalence principle of the present invention.

These sequences were used as examples inter alia in order to illustrate the benefits of the supravalence principle.

FIG. 25 describes the results of monomeric EPO mimetic peptides in comparison with EPO. FIG. 25 includes a plot of actual absorbance data documenting the absolute difference between peptides in general and EPO in this assay.

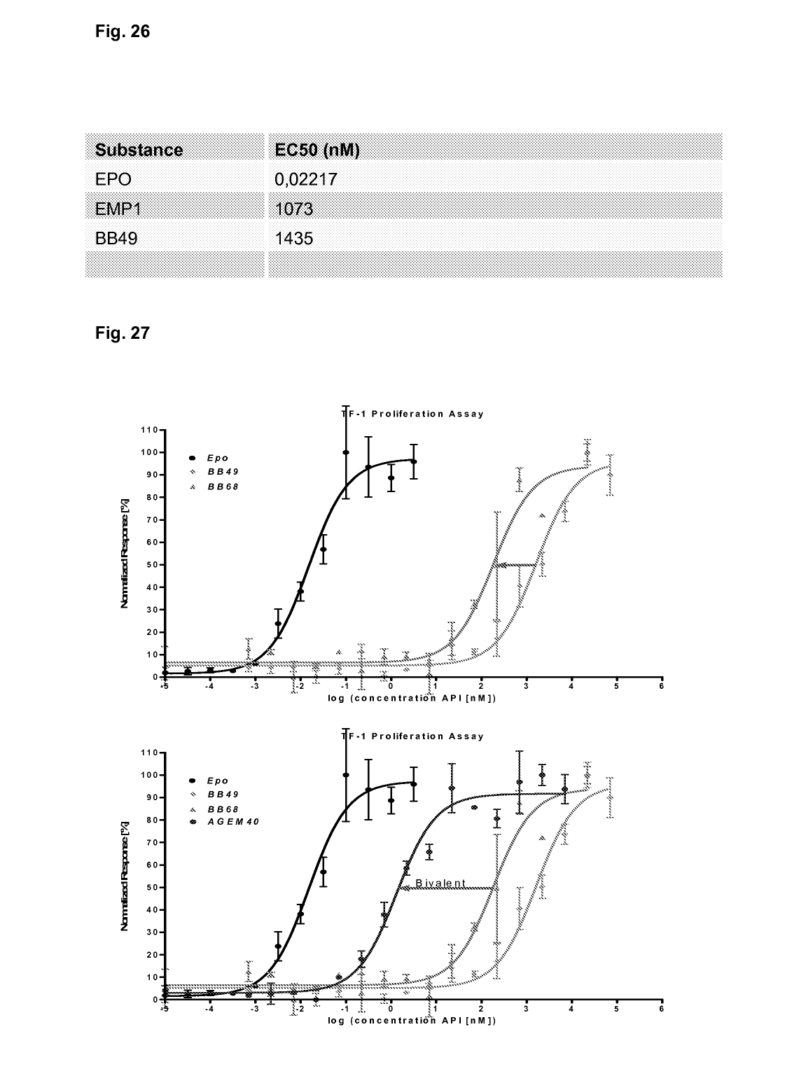

FIG. 26 gives the EC50 values calculated from the fitted normalized plots.

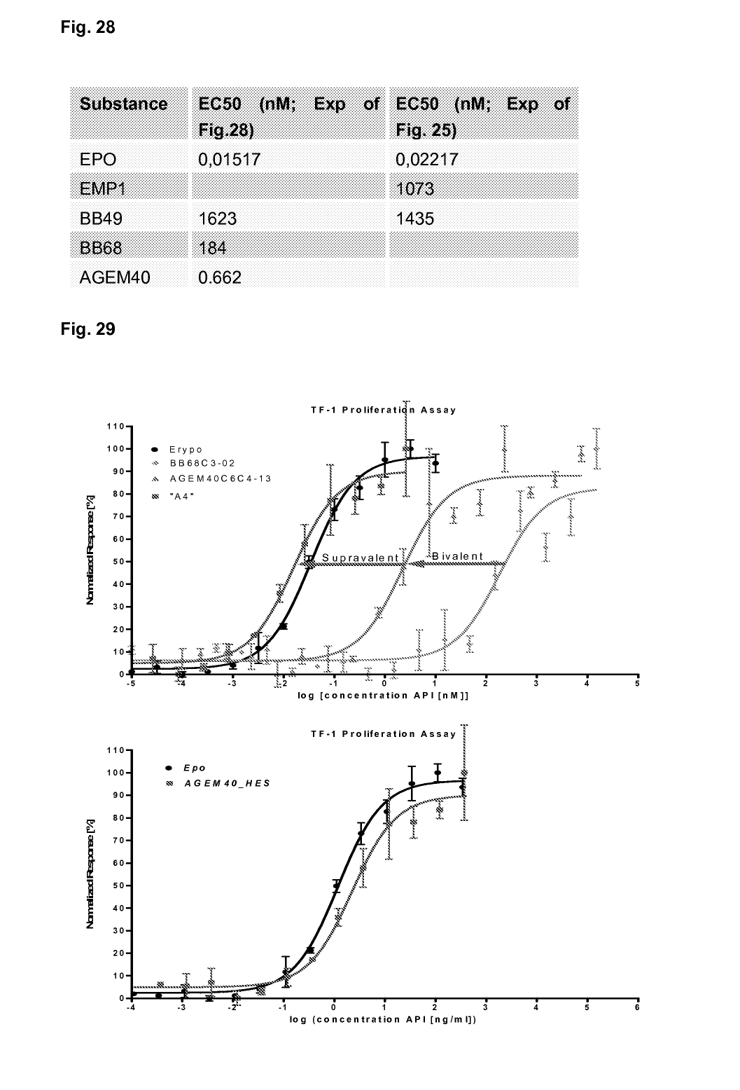

FIG. 27 shows the improved effect of B868 compared to B849. Using the optimized BB68 as building block for creating a peptide unit according to the present invention, the effect was improved by two additional orders of magnitude. This is documented in FIG. 27 and the corresponding Table shown in FIG. 28.

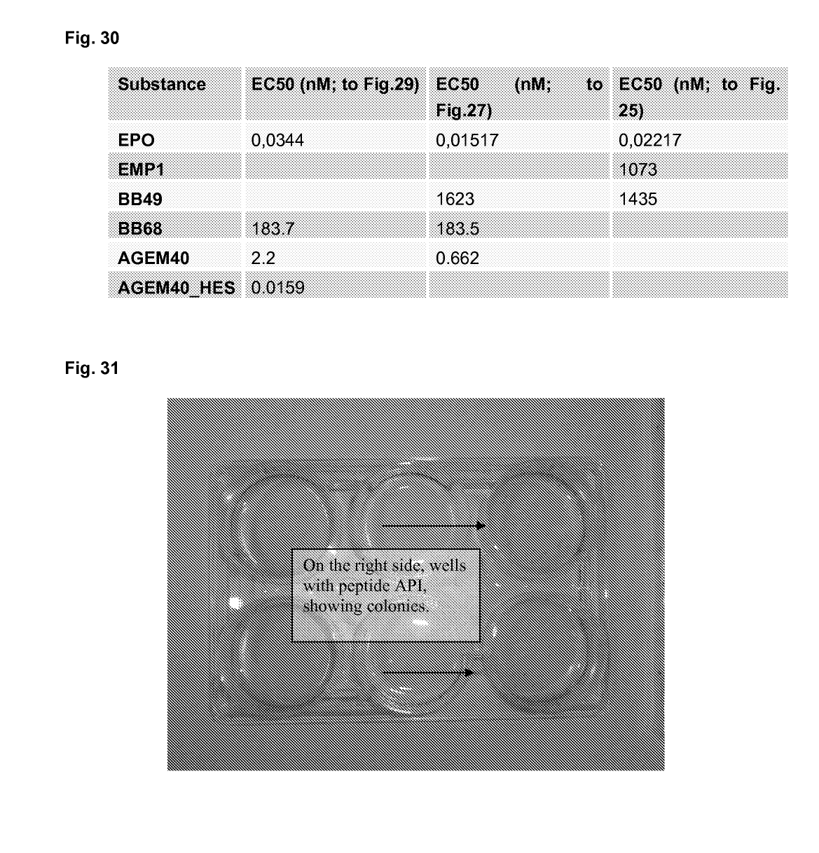

The dimeric peptide units were then coupled to the macromolecular carrier HES at an optimized density. The resulting API is at least equipotent to EPO on molar comparison and very close to EPO on mass comparison (see FIG. 29 and FIG. 30 below).

FIG. 30 and the Figures and Tables before clearly demonstrate the great potency of the supravalence concept. Keeping the accuracy in mind, which can be achieved with a cell culture assay, the achieved API is at least equipotent to EPO in vitro. It is thus superior to any known EPO-mimetic peptide API not employing the supravalence concept.

### Bone Marrow Assays

Bone marrow contains hematopoietic stem cells with a potential so self-renew and to develop into all types of blood cells. In addition, bone marrow contains committed progenitor cells capable of developing into one or several blood cell lineages. Among those progenitor cells, some develop into erythrocytes (erythroid progenitors).

Progenitor cells can be demonstrated by plating bone marrow cells in methylcellulose-based semi-solid media. In the presence of an appropriate cytokine cocktail progenitor cells proliferate and differentiate to yield a colony of cells of a certain lineage. Myeloid progenitors develop into granulocytic colonies (derived from a CFU-G), monocytic colonies (from a CFU-M), or mixed granuocytic-monocytic colonies (from a CFU-GM). Erythroid progenitors develop into a colony of erythrocytes (red cells). Depending on the size of the erythroid colony, the progenitor cells are called BFU-E (yielding colonies of 200 cells or more) of CFU-E (yielding colonies of less than 200 cells). Progenitor cells in an earlier stage of commitment can develop into mixed granulocytic-erythroid-monocytic-megakaryocytic colonies. These early progenitors are called CFU-GEMM.

EPO stimulates the development of erythroid colonies from BFU-E or CFU-E, if certain different cytokines are present as well. Without EPO no erythroid colonies can develop. Outgrowth of erythroid colonies from a homogenous batch of bone marrow cells in methylcellulose, therefore, is a measure for EPO activity.

Since the abovementioned processes are very similar if not identical to the processes which occur in the bone marrow in vivo, they are an excellent predictor of EPO-like activity.

### Design of Bone Marrow Assays

Human bone marrow cells (commercially available from Cryosystems, serologically checked) are thawed from cryovials, and plated in methylcellulose media with a given background of cytokines (but without EPO) at a fixed cell density. EPO or EPO-mimetic peptide is added at varying concentrations. Cultures are incubated for 12-14 days at 37 C. Then, the numbers of myeloid and erythroid colonies are enumerated by microscopic inspection.

### End Points of Bone Marrow Assays:

1. Premisses: Cultures without EPO should only yield myeloid (white) but not erythroid (red) colonies. Cultures with EPO should yield a concentration-dependent increase in red cell colonies, and a concentration-dependent increase in the sizes of the red cell colonies.

2. A peptide shows EPO-mimetic activity if it causes a concentration-dependent increase in red cell colonies, and a concentration-dependent increase in the sizes of the red cell colonies. However, a peptide should not interfere with the numbers of myeloid colonies obtained.

### Results of Bone Marrow Assays

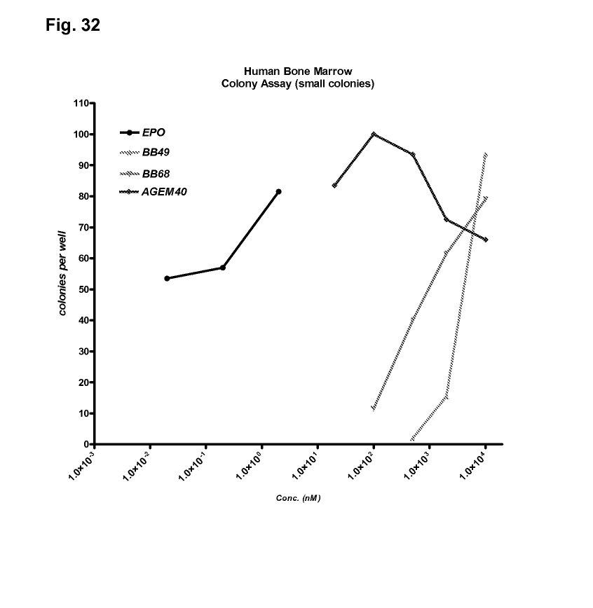

The proline modified EPO mimetic peptides described above did not stimulate the formation of myeloid colonies, but showed significant activity on the formation of red colonies. Qualitatively, this is shown in the FIG. 31 in a photograph of a culture plate, while counting of colonies is documented in FIG. 32.

### REFERENCES

Wrighton N C, Balasubramanian P, Barbone F P, Kashyap A K, Farrell F X, Jolliffe L, Barrett R W, Dower W J (1997) Increased potency of an erythropoietin peptide mimetic through covalent dimerization. Nature Biotechnology 15:1261-1265
Wrighton N C, Farrell F X, Chang R, Kashyap A K, Barbone F P, Mulcahy L S, Johnson D L, Barrett R W, Jolliffe L K, Dower W J (1996) Small Peptides as Potent Mimetics of the Protein Hormone Erythropoietin, Science 273:458-463
Johnson, D. L., F. X. Farrell, et al. (1997). “Amino-terminal dimerization of an erythropoietin mimetic peptide results in increased erythropotietic activity.” Chemistry and Biology 4: 939-950,
Haag R, Sunder A, Stumbé J F, J. Am. Chem. Soc. (2000), 122, 2954.
Roberts, M. J., M. D. Bentley, et al. (2002). “Chemistry for peptide and protein PEGylation.” Advanced Drug Delivery Review 54(4): 459-476.
Zalipsky S, Qazen, 8, Walker II J A, Mullah N, Quinn Y P, (1999) “New detachable poly (ethylene glycol) conjugates: Cysteine-cleavable lipopolymers regenerating natural phospholipid, diacyl phosphatidylethanolamine, Bioconjug. Chem. 10: 703-707.
Zhao, X. et al (1997), “Novel Degradable Poly(ethylene glycol) esters for drug delivery.” In “Poly(ethylene glycol) chemistry and biological applications; Harris J M, Zalipsky, S. Eds.; ACS Symposium Series 680; American Chemical Society: Washington D.C., 1997; 458-472.

1. A compound binding to a target molecule, comprising
(i) at least two peptide units wherein each peptide unit comprises at least two domains with a binding capacity to a target;
(ii) at least one polymeric carrier unit;
wherein said peptide units are attached to said polymeric carrier unit.

2-32. (canceled)
